(19) United States
(12) Patent Application Publication
(10) Pub. No.: US 2026/0137713 A1
(43) Pub. Date: May 21, 2026

(54) POLYNUCLEOTIDES FOR SILENCING TRANSCRIPT VARIANT 1 OF ASSEMBLY FACTOR FOR SPINDLE MICROTUBULES AND APPLICATIONS THEREOF

(71) Applicant: TAIPEI MEDICAL UNIVERSITY, TAIPEI CITY (TW)

(72) Inventor: Kun-Chih Kelvin TSAI, Taipei City (TW)

(73) Assignee: TAIPEI MEDICAL UNIVERSITY, TAIPEI CITY (TW)

(21) Appl. No.: 19/119,293

(22) PCT Filed: Oct. 9, 2023
(86) PCT No.: PCT/US2023/034750

§ 371 Date: Apr. 8, 2025

Related U.S. Application Data
(60) Provisional application No. 63/414,461, filed on Oct. 8, 2022.

(51) Int. Cl. A61K 31/712; A61K 9/00; A61K 9/51; A61K 31/7105; A61K 31/713; A61P 35/04; C12N 15/113

(52) U.S. Cl. CPC A61K 31/712; A61K 9/0019; A61K 9/51; A61K 31/7105; A61K 31/713; A61P 35/04; C12N 15/113; C12N 2310/14; C12N 2310/321

(57) ABSTRACT

The present disclosure relates to polynucleotides and methods of inhibiting the activities of development-associated pathways, including the Wnt, Hedgehog, and Notch pathways, and the invasive capacity of malignant cells to treat primary or secondary solid tumors.

### CROSS-REFERENCES TO RELATED APPLICATIONS

This application claims the benefit of U.S. Provisional Application No. 63/414,461, filed on 8 Oct. 2022, the disclosure of which is hereby incorporated by reference in its entirety for all purposes.

### FIELD OF THE INVENTION

The present disclosure relates to a field of cancer treatment. Particularly, the present disclosure provides polynucleotides silencing assembly factor for spindle microtubules (ASPM) and their uses in treating cancers.

### BACKGROUND OF THE INVENTION

Development signaling pathways, including Wnt, Notch, and Hedgehog, regulate stem cell homeostasis in adult tissues, and are frequently dysregulated during the malignant transformation processes, giving rise to the stemness properties of tumors. Canonical Wnt signaling like β-catenin has been implicated in epithelial tissue homeostasis by maintaining stem cell proliferation and migration, especially in the intestine, mammary gland, and skin. Increasing evidence suggests that the Wnt-regulated self-renewal processes in tissue progenitor and stem cells are usurped by cancer cells to facilitate malignant progression. Consistently, there are now compelling data supporting the role of the Wnt/β-catenin signaling pathway in sustaining the cancer stem cell (CSC) phenotype in solid tumors. There is increasing evidence that the Wnt signaling plays an essential role in the metastatic colonization of cancer cells, and these experimental studies emphasize the interaction between microenvironmental factors and CSCs. For instance, stroma-cell-derived extracellular matrix (ECM) protein periostin recruits Wnt ligands and thereby activates Wnt signaling in CSCs in the metastatic colonization of breast cancer.

Cancer invasiveness and distant metastasis are the major causes of patient mortality, and understanding it is critical to improving the outcome of patients with solid tumors. Invadopodia or podosomes are transient actin-based protrusions present on immune cells and certain cancer cells that mediate focal degradation of extracellular matrix (ECM) by the localized proteolytic activity of proteases. Aside from directly degrading ECM, invadopodia also facilitate cancer cell invasiveness by initiating crosstalk with the ECM and exerting physical force toward the surrounding stroma to open micron-sized channels (Iizuka, S, Leon, R. P., Gribbin, K. P., Zhang, Y., Navarro, J. Smith, R., Devlin, K., Wang, L. G., Gibbs, S. L., Korkola, J., et al. (2020). Crosstalk between invadopodia and the extracellular matrix. Eur J Cell Biol 99, 131122). Invadopodia have been shown to exist in vivo and are thus hypothesized to play a critical role in the establishment of distant metastasis by facilitating the process of intravasation and extravasation. In accordance with these observations, a series of functional studies demonstrated that the inhibition of invadopodia regulators, including SRC, PDGFR-α, TKS-5, or a specific variant of MENA (MENA^{INV}), could inhibit metastasis in various cancer models (Eckert, M. A., Lwin, T. M, Chang, A. T., Kim, J., Danis, E., Ohno-Muchado, L., and Yang, J. (2011). Twist1-induced invadopodia formation promotes tumor metastasis. Cancer Cell 19, 372-386; Weidmann, M. D., Surve, C. R., Eddy, R. J., Chen, X., Gertler, F. B., Sharma, V. P., and Condeelis, J. S. (2016). Mena(INV) dysregulates cortactin phosphorylation to promote invadopodium maturation. Sci Rep 6, 36142).

Thus, there is a need for drugs and methods for treating and preventing cancers and metastasis thereof.

### SUMMARY OF THE INVENTION

The present disclosure relates to polynucleotides and methods of inhibiting the activities of development-associated pathways, including the Wnt, Hedgehog, and Notch pathways, and the invasive capacity of malignant cells to treat primary or secondary solid tumors.

The present disclosure provides a polynucleotide, comprising a nucleotide sequence complimentary to the mRNA of the ASPM gene with a nucleotide sequence shown in SEQ ID NO:1; or b) a nucleotide sequence comprising a contiguous segment having at least 70%, at least 80%, or at least 90% sequence identity to the nucleotide sequence complimentary to SEQ ID NO:1.

In one embodiment, the polynucleotide comprises a nucleotide sequence complementary to the miRNA encoded by exon 18 of the transcript variant 1 of the human ASPM gene with a nucleotide sequence shown in SEQ ID NO:3 or a nucleotide sequence comprising a contiguous segment having at least 70%, at least 80%, or at least 90% sequence identity to the nucleotide sequence complimentary to SEQ ID NO:3.

In some embodiments, the polynucleotides as described herein are RNAi. Examples of the RNAi include, but are not limited to shRNA, siRNA or dsRNA.

Certain embodiment of the polynucleotide of the present disclosure is an siRNA molecule, wherein said siRNA molecule comprises: (a) a duplex region; and (b) either no overhang regions or at least one overhang region, wherein each overhang region contains six or fewer nucleotides, wherein the duplex region consists of a sense region and an antisense region, wherein said sense region and said antisense region together form said duplex region and said antisense region and said sense region each 15-30 nucleotides in length and said antisense region comprises a sequence that is the complement of a sequence selected from SEQ ID NO: 3, 5, 7 or 9.

In some embodiments, the siRNA molecule as described herein has the antisense region and the sense region are each 15-25, 15-20 or 15-18 bases in length.

In some embodiments, the siRNA molecule is a chemically synthesized double stranded siRNA molecule, wherein: (a) each strand of said double stranded siRNA molecule is between 15 and 30 nucleotides in length; and (b) one strand of said siRNA molecule comprises a sequence that is the complement of a sequence selected from SEQ ID NO: 3, 5, 7 or 9.

In some embodiments, the siRNA molecule described herein comprises a sense strand and an antisense strand, wherein said sense strand comprises at least 15 contiguous nucleotides differing by no more than 3 nucleotides from SEQ ID NO: 4, and said antisense strand comprises at least 15 contiguous nucleotides differing by no more than 3 nucleotides from SEQ ID NO: 5.

In a certain embodiment, the siRNA molecule comprises a sense strand and an antisense strand, wherein said sense strand comprises a nucleotide sequence as set forth in SEQ ID NO:4 and said antisense strand comprises a nucleotide sequence as set forth in SEQ ID NO:5.

In some embodiments, the siRNA molecule described herein comprises a sense strand and an antisense strand forming another double-stranded RNA dimer, wherein said sense strand comprises at least 15 contiguous nucleotides differing by no more than 3 nucleotides from SEQ ID NO: 6, and said antisense strand comprises at least 15 contiguous nucleotides differing by no more than 3 nucleotides from SEQ ID NO:7.

In a certain embodiment, the siRNA molecule comprises a sense strand and an antisense strand, wherein said sense strand comprises a nucleotide sequence as set forth in SEQ ID NO:6 and said antisense strand comprises a nucleotide sequence as set forth in SEQ ID NO:7.

In some embodiments, the siRNA molecule described herein comprises a sense strand and an antisense strand forming another double-stranded RNA dimer, wherein said sense strand comprises at least 15 contiguous nucleotides differing by no more than 3 nucleotides from SEQ ID NO: 8, and said antisense strand comprises at least 15 contiguous nucleotides differing by no more than 3 nucleotides from SEQ ID NO:9.

In a certain embodiment, the siRNA molecule comprises a sense strand and an antisense strand, wherein said sense strand comprises a nucleotide sequence as set forth in SEQ ID NO:8 and said antisense strand comprises a nucleotide sequence as set forth in SEQ ID NO:9.

In some embodiments, the siRNA molecule has at least one overhang region or has no overhang region.

In some embodiments, the one or two of the sense strand and the antisense strand can be further modified as modified siRNA. Examples of the modified nucleotide include, but are not limited to, a 2′-O-methyl modified nucleotide, a 2′-fluorophoramidate, a 3′-terminal deoxy-thymine nucleotide, a non-natural base comprising a nucleotide, a nucleotide comprising a 5′phosphorothioate group, and a terminal nucleotide linked to a cholesteryl derivative and a dodecanoic acid bisdecylamide group.

In some further embodiments, the modified siRNA comprises 10% to about 30% of the nucleotides in the double stranded region comprise 2′-O-methyl (2′OMe) nucleotides, comprises 2′OMe nucleotides on both strands of the modified siRNA.

The present disclosure provides a pool of siRNA molecules, wherein said pool comprises one or more the first siRNA molecule or a modified siRNA molecule thereof, the second siRNA molecule or a modified siRNA molecule thereof, the third siRNA molecule or a modified siRNA molecule thereof and the fourth siRNA molecule or a modified siRNA molecule thereof,
wherein
said first siRNA molecule is a chemically synthesized double stranded siRNA molecule, wherein: (a) each strand of said double stranded siRNA molecule is between 15 and 30 nucleotides in length; and (b) one strand of said siRNA molecule comprises a sequence that is the complement of a sequence selected from SEQ ID NO:3;
said second siRNA molecule is a chemically synthesized double stranded siRNA molecule, wherein: (a) each strand of said double stranded siRNA molecule is between 15 and 30 nucleotides in length; and (b) one strand of said siRNA molecule comprises a sequence that is the complement of a sequence selected from SEQ ID NO:5;
said third siRNA molecule is a chemically synthesized double stranded siRNA molecule, wherein: (a) each strand of said double stranded siRNA molecule is between 15 and 30 nucleotides in length; and (b) one strand of said siRNA molecule comprises a sequence that is the complement of a sequence selected from SEQ ID NO: 7; and
said fourth siRNA molecule is a chemically synthesized double stranded siRNA molecule, wherein: (a) each strand of said double stranded siRNA molecule is between 15 and 30 nucleotides in length; and (b) one strand of said siRNA molecule comprises a sequence that is the complement of a sequence selected from SEQ ID NO: 9.

In some embodiments, the siRNA molecules in the pool comprises one of more the second siRNA molecule or a modified siRNA molecule thereof, the third siRNA molecule or a modified siRNA molecule thereof and the fourth siRNA molecule or a modified siRNA molecule thereof.

In some embodiments, the siRNA molecules in the pool comprises a sense strand and an antisense strand forming another double-stranded RNA dimer, wherein said sense strand comprises at least 15 contiguous nucleotides differing by no more than 3 nucleotides from SEQ ID NO: 4, 6 or 8, and said antisense strand comprises at least 15 contiguous nucleotides differing by no more than 3 nucleotides from SEQ ID NO: 3, 5 or 9.

In a further embodiment, the siRNA molecule in the pool comprises a sense strand and an antisense strand, wherein said sense strand comprises a nucleotide sequence as set forth in SEQ ID NO: 4 and said antisense strand comprises a nucleotide sequence as set forth in SEQ ID NO:5.

In a further embodiment, the siRNA molecule in the pool comprises a sense strand and an antisense strand, wherein said sense strand comprises a nucleotide sequence as set forth in SEQ ID NO: 6 and said antisense strand comprises a nucleotide sequence as set forth in SEQ ID NO:7.

In a further embodiment, the siRNA molecule in the pool comprises a sense strand and an antisense strand, wherein said sense strand comprises a nucleotide sequence as set forth in SEQ ID NO: 8 and said antisense strand comprises a nucleotide sequence as set forth in SEQ ID NO:9.

The present disclosure provides a pharmaceutical composition comprising at least one the polynucleotide described herein and a pharmaceutical carrier, diluent and/or adjuvant.

The present disclosure provides a nanoparticle comprising the polynucleotide described herein or any mixture thereof and lipid nanoparticle (LNPs), liposome, micelle, virosome or nucleic acid complex.

The present disclosure also provides a method for inhibiting growth, loco-regional spreading, and distant metastasis of a malignant tumor and/or treating a solid tumor and/or tumor metastasis in a subject, comprises administrating the polynucleotide described herein or any mixture thereof, the pool of siRNA molecules described herein, the pharmaceutical composition described herein or the nanoparticle described herein to the subject.

In some embodiments, the human solid cancer is breast cancer, non-small-cell lung carcinoma (NSCLC), PDAC, the scirrhous subtype of gastric adenocarcinoma, and the “stem/serrated/mesenchymal (SSM)” molecular subtype of colorectal cancer (CRC).

### BRIEF DESCRIPTION OF THE DRAWINGS

FIGS. 1 (A) and (B) show that ASPM expression correlates with breast cancer metastasis. (A) shows that ASPM is the top-ranked Wnt-related factor associated with metastasis in breast cancer in the Pawitan et al. data set. (B) shows Kaplan-Meier survival curve comparing distant metastasis-free survival of breast cancer patients stratified according to the ASPM expression level queried from KM Plotter (http://kmplot.com/analysis/index.php?p=service) and SurvExpress (http://bioinformatica.mty.itesm.mx:8080/Biomatec/SurvivaX.jsp). The P-value is calculated using the log-rank test.

FIGS. 2 (A) and (B) show genetic knockdown (knockdown) of ASPM expression reduced the invasive capability of breast cancer cells. The ASPM-v1 expression was stably downregulated in HCC-1954 and MDA-MB-436 breast cancer cells using lentivirus-mediated knockdown. The invasive capacity of cancer cells with control- or ASPM-knockdown across recombinant basement membrane was examined using a modified Boyden chamber invasion assay. (A) shows representative immunofluorescence images of the invaded cells, with cell nuclei stained with CYTOX-green. Scale bars=100 μm. (B) shows the numbers of invaded cells as in (A). Data are mean±SEM. ***P<0.001.

FIGS. 3 (A) and (B) include several panels related to the effect of the genetic knockdown (knockdown) of ASPM expression on distant metastasis in breast and pancreatic cancers. (A) shows representative bioluminescence (BLI) of the metastatic pulmonary tumors at the indicated time (weeks; wk) after cell inoculation. (B) shows the BLI signals in (A) shown as normalized photon counts as a function of time. Data are shown as means±SEM (n=5 mice per group). **P≤0.01 compared to control IgG.

FIGS. 4 (A) and (B) include several panels relating to the elevated expression level of ASPM in micro-metastatic cancers in patient-derived xenograft (PDX) models of breast cancer progression. (A) shows a representative flow cytometry plot showing patterns of ASPM staining of CD298-positive cancer cells in primary tumor and pulmonary micro-metastases. (B) shows elevated expression of ASPM in CD298-positive cancer cells in the micro-metastatic lesions in two PDX models (BR1282 and BR1474). Data are mean±SEM. **P<0.01.

FIG. 5 shows the putative domain architectures of human ASPM isoform 1 and isoform 2 proteins. CH, calponin-homology; ARM, armadillo; IQ, isoleucine, and glutamine. The region encoded by the exon 18, which carries 67 IQ domains, is highlighted.

FIGS. 6 (A) to (E) includes several panels relating to the expression pattern of ASPM isoforms in normal and cancer tissues. (A) shows the immunohistochemical (IHC) staining pattern of ASPM isoform 1 (ASPM-i1) and ASPM isoform 2 (ASPM-i2) in representative human breast cancer tissues and normal breast tissues. Scale bar, 50 μm. (B) Bar graphs showing the distribution of the single-cell staining intensities (1+ to 3+) of ASPM-i1 and ASPM-i2. Data are means±SEM (n=40). ***P<0.001. (C) shows the IHC staining pattern of ASPM-i1 in the invasive front of a representative human breast cancer tissue. Scale bar, 100 μm. (D) Bar graphs showing the percentage of the cancer cells with a moderate (2+)-to-high (3+) staining intensity of ASPM-i1. Data are means±SEM (n=46). ***P<0.001. (E) shows the IHC staining pattern of ASPM-i1 and ASPM-i2 in representative normal, hepatitis, and cirrhotic liver tissues and HCC tissues. Scale bar, 50 μm. (F) Bar charts showing the distribution of the single-cell staining intensities (1+ to 3+) of ASPM-i1 and ASPM-i2 in normal liver tissues (n=24), hepatitis liver tissues (n=10), cirrhotic liver tissues (n=50), and HCC tissues (n=111). Data are mean±SEM. **P<0.01; ***P<0.001 versus normal liver.

FIG. 7 shows that ASPM isoform 1 (ASPM-i1) immunoprecipitated with the PAR-planar cell polarity proteins, including PAR-6α, PAR-6β, disheveled-2 (DVL2), CDC42, and SMURF1, in invasive breast cancer MDA-MB-436 cells. The non-invaded cancer cells are included as a control,

FIGS. 8 (A) and (B) include several panels relating to the specific interaction of ASPM isoform 1 (ASPM-i1) with the PAR/PCP proteins DVL2, PAR6β, and CDC42 in the invadopodia of cancer cells. (A) shows confocal imaging showing the colocalization of ASPM-i1 (red), DVL2, PAR6β, CDC42, or N-WASP (blue), and the invadopodial marker cortactin (green) in the invadopodia of breast cancer MDA-MB-436 cells. Scale, 10 μm. (B) shows ASPM-i1 specifically immunoprecipitated with DVL2, PAR6β, CDC42, and N-WASP in the invadopodia of invasive MDA-MB-436 cells. Cortactin and TKS5 are included as invadopodia markers.

FIGS. 9 (A) to (C) include several panels relating to the effects of the genetic knockdown (knockdown) of ASPM variant 1 (ASPM-v1; which encodes “ASPM isoform 1” or “ASPM-i1”) expression on the invadopodia formation and the invasive capacity of breast cancer cells. Breast cancer MDA-MB-436 cells were lentivirally transduced with a small hairpin RNA (shRNA) that specifically targets the transcript variant 1 of the ASPM gene (ASPM-v1 shRNA) to knock down the expression of ASPM-v1. (A) shows the effect of the isoform-specific of ASPM-v1 shRNAs, including ASPM. e18 shRNA #1 and ASPM.c18 shRNA #4, on the protein abundance levels of ASPM-i1 in MDA-MB-436 cells. (B) shows that knockdown of ASPM-v1 expression attenuated the recruitment of DVL2, PAR6β, CDC42, and membrane-type matrix metalloproteinase (MT1-MMP) to the invadopodia of MDA-MB-436 cells. Shown are immunoblots of the indicated proteins in the invadopodial lysates of MDA-MB-436 cells lentivirally transduced with control shRNA or ASPM. e18 shRNA #4. (C) shows that knockdown of ASPM-v1 expression reduced the invasive capacity of MDA-MB-436 cells as examined using a modified Boyden chamber invasion assay. Left, representative immunofluorescence images of the invaded cells, with cell nuclei stained with CYTOX-green. Scale bar, 50 μm. Right, quantification of the invaded cells. Data are mean±SEM. ***P<0.001,

FIG. 10 shows that the genetic knockdown (knockdown) of ASPM variant 1 (ASPM-v1) expression affects multiple development- and stemness-associated pathways. Relative luciferase reporter activity of the indicated development- and stemness-associated signaling pathways in 293T cells that were knocked down of ASPM-v1 using lentivirus-mediated transduction of ASPM.18 shRNA #4 or those infected with a control shRNA lentivirus and transduced with the respective reporter constructs in the Cignal Finder Stem Cell & Differentiation 10-Pathway Reporter Array (n=3 per group). *P<0.05; **P<0.01; ***P<0.001 compared to control shRNA.

FIG. 11 includes two panels relating to the selection of optimized siRNAs targeting ASPM variant 1 (ASPM-v1). Shown are the transcript levels of ASPM-v1 in MDA-MB-436 cells transduced with an equal total amount (50 nM for all siRNAs) of the three top-ranked siRNA as described in Step 4 or the different combinations thereof using qRT-PCR analysis (mean±SEM; n=3). *P<0.5; **P<0.01; ***P<0.001 compared with NT siRNA.

FIGS. 12 (A) and (B) include two panels relating to the effect of the lipid nanoparticle (LNP)-formulated small interfering RNAs mixture (siRNA) specifically targeting the mRNA region of exon 18 of ASPM variant 1 (LNP-siASPM-v1). (A) shows that the treatment of breast cancer MDA-MB-436 cells with an increasing concentration (0-50 nM) of LNP-ASPM-v1 siRNA could dose-dependent reduce the transcript levels of ASPM-v1. (B) shows that the treatment of MDA-MB-436 cells with LNP-siASPM-v1 dose-dependent reduced the protein abundance levels of ASPM isoform 1 (ASPM-i1) but not the isoform 2 of the ASPM protein (ASPM-i2) NT siRNA, non-target siRNA.

FIGS. 13 (A) and (B) show the effect of LNP-siASPM-v1 on the invadopodia formation and the invasive capacity of cancer cells. (A) shows the inhibitory effect of the LNP-formulated ASPM-v1-specific siRNA (LNP-siASPM-v1) on the invadopodia formation of cancer cells. Shown on the left are confocal views of the cortactin “F-actin” puncta (yellow), which represent the cross-sections of the downwardly protruding invadopodium, in MDA-MB-436 cells treated with LNP-siASPM-v1 or LNP-non-target control siRNA (NT siRNA) (both at 100 nM×72 hours). Right, the number of cortactin “F-actin” invadopodia per cell. Scale bar, 10 μm. Data are mean±SEM. ***P<0.001. (B) shows that treatment of MDA-MB-436 cells substantially inhibited the invasive capacity of the cells as examined using a dual-chamber invasion assay. Shown on the left are representative immunofluorescence images of the invaded cells, with cell nuclei stained with CYTOX-green (green). Scale bars=500 μm. Right, the numbers of invaded cells. Data are mean±SEM. **P<0.01.

FIGS. 14 (A) to (C) include several panels relating to the effect of the LNP-formulated ASPM variant 1-specific siRNA (LNP-siASPM-v1) on the Wnt activity, and the stemness property and the tumorigenicity of hepatocellular carcinoma (HCC) cells. (A) shows the fold Wat-specific luciferase expression in HuH-1 cells that were treated with LNP-siASPM-v1 or LNP-non-target control siRNA (NT siRNA) (both at 100 nM×72 hours), and then WNT3A (250 ng/ml×16 hours). (B) shows the percentage of aldehyde dehydrogenase (ALDH)-positive cell population (representing cancer stemness in HCC) in HuH-1 cells treated with LNP-siASPM-v1 or LNP-non-target control siRNA as in (A). Data are shown as means±SEM (n=3). ***P≤0.001 compared to LNP-NT siRNA in (A) and (B). (C) shows the inhibitory effect of LNP-siASPM-v1 on the tumorsphere-formation ability of HCC cells. HuH-1 cells were treated with LNP-siASPM-v1 or LNP NT siRNA as in (A). The cells were then cultured in serum-free and nonadherent culture plates for ten days. Shown are representative phase-contrast images of the resultant tumorspheres. Scale bars, 50 μm. Right, limiting dilution assay demonstrating the tumorsphere formation efficacy of the cells. Data are means±SEM (n=4 in each group).

FIGS. 15 (A) to (D) include several panels relating to the pharmacodynamic studies of systemic LNP-formulated ASPM variant 1-targeted siRNA therapy LNP-siASPM-v1 in an orthotopic mouse model of triple-negative breast cancer (TNBC). (A) shows that GFP- and firefly luciferase (FF-Luc)-expressing breast cancer MDA-MB-436 cells were injected orthotopically into the mammary fat pads of immunodeficient NOD/SCID mice. Ten days following cell inoculation, when the tumors were detectable by bioluminescence (BLI), the tumor-bearing mice received intravenous injections of Cy5-labeled LNP-10 siASPM-v1 (100) μg per mouse [approximately 4 mg/kg]) every three days for a total of 2 doses. The tumors were removed at the indicated time following the second injection for cell dissociation, and the GFP cancer cells were sorted for the subsequent analyses. (B) shows representative FACS plots showing patterns of GFP and Cy5 staining of cancer cells described in (A) with the frequency of Cy5⁺GFP⁺ cell population shown. Right, the percentage of Cy5⁺ cancer cells at different times following the completion of the 15 treatment. T_{1/2}, half-life. (C) Representative confocal imaging showing the accumulation of Cy5 (red)-labeled siRNA in the cytoplasm of breast tumor cells following intravenous LNP-ASPM-v1 siRNA treatments. The cytoplasm was marked by staining F-actin with Phalloidin (green). Cell nuclei were counterstained with DAPI (blue). Scale bar, 5 μm (D) Representative immunoblots showing the specific knockdown effect of the systemic LNP-siASPM-v1 therapy on the protein abundance level of ASPM-isoform 1 (ASPM-i1) in the treated tumors. Right, the relative protein abundance level of ASPM-i1 in the tumor-bearing mice treated with LNP-non-target control siRNA (NT siRNA) or LNP-siASPM-v1. Data are means±SEM (n=3 in each group). *P<0.05.

FIGS. 16 (A) to (C) include several panels relating to the anti-metastasis efficacy of ASPM variant 1 (ASPM-v1)-targeted siRNA therapy in an orthotopic mouse model of triple-negative breast cancer (TNBC). (A) shows TNBC MDA-MB-436 cells that stably express firefly luciferase (FF-Luc) were injected orthotopically into the mammary fat pads of immunodeficient NOD/SCID mice. Two weeks following cell inoculation, when the tumors were detectable by BLI, the tumor-bearing mice received intravenous (IV; 2 mg/kg or 4 mg/kg every three days; 6 doses in total) injections of LNP-siASPM-v1 or LNP-non-target control siRNA (NT siRNA). (B) shows representative BLI of primary or metastatic tumors (lung window) 30 at the indicated time following initiation of the treatments in (A). (C) shows the tumor bulk in (B) quantified as BLI normalized photon counts as a function of time. Data are shown as means±SEM (n=5 mice per group). **P≤≤0.01; ***P≤0.001 compared to LNP-NT siRNA.

FIGS. 17 (A) to (C) include several panels relating to the anti-metastasis effect of systemic ASPM variant 1 (ASPM-v1)-targeted siRNA therapy in distant metastasis of triple-negative breast cancer (TNBC). (A) shows that NOD/SCID mice were tail vein injected with firefly luciferase (FF-Luc)-expressing MDA-MB-436 cells. Twenty-four hours following cell inoculation, the mice received intravenous injections of LNP-siASPM-v1 (100 μg per mouse [approximately 4 mg/kg] every three days; 6 doses in total) or an LNP-non-target control siRNA (NT siRNA), and the distribution of metastatic tumors was monitored by BLI. (B) shows representative BLI of metastatic tumors at the indicated time following cell inoculation. (C) shows the BLI signals in (B) shown as normalized photon counts as a function of time. Data are shown as means±SEM (n=5 mice per group). **P<0.01 compared to LNP-NT siRNA.

FIGS. 18 (A) and (B) include several panels relating to the anti-tumor efficacy of intratumoral ASPM variant 1 (ASPM-v1)-targeted siRNA therapy in mouse xenograft models of hepatocellular carcinoma (HCC). (A) shows HuH-1 cells that stably express GFP and firefly luciferase (FF-Luc) were injected subcutaneously in the flanks of immunodeficient NOD/SCID mice. LNP-siASPM-v1 or LNP-non-target control siRNA (NT siRNA; 0.8 mg/kg or 2 mg/kg of oligonucleotide per intra-tumoral injection every three days: 3 doses in total) with or without the concurrent intraperitoneal (IP) injections of sorafenib (15 mg/kg/day for a consecutive ten days) or vehicle was administered to the tumor-bearing mice. Shown are representative bioluminescence (BLI) of tumors at the indicated time after cell inoculation. (B) shows the tumor bulk quantified as BLI normalized photon counts as a function of time in the tumor-bearing mice described in (A). Data are shown as means±SEM (n=8 mice per group). *P≤0.05 compared to LNP-NT siRNA. Data are shown as means±SEM (n=3 mice per group).

FIGS. 19 (A) and (B) include several panels relating to the anti-tumor efficacy of ultrasound-guided intratumoral ASPM variant 1 (ASPM-v1)-targeted siRNA therapy in the orthotopic mouse xenograft model of hepatocellular carcinoma (HCC). (A) shows the HuH-1 cells were injected into the left lobes of the livers of NOD/SCID mice. Two weekly following cell inoculation, the tumor-bearing mice received ultrasound-guided intra-tumoral injections of LNP-siASPM-v1 or LNP-non-target control siRNA (NT siRNA: 0.8 mg/kg every three days; 3 injections in total). (B) shows representative photographs of the tumors in (A) at the study end point. Right, tumor volume plotted over time. Data are shown as means±SEM (n=5 mice per group). **P≤0.01 compared to LNP-NT siRNA.

FIGS. 20 (A) to (E) include several panels relating to the anti-tumor efficacy of the systemic ASPM variant 1 (ASPM-v1)-targeted siRNA in an orthotopic mouse model of hepatocellular carcinoma (HCC). (A) shows that GFP- and FF-Luc-expressing HuH-1 cells were injected into the left lobe of the livers of immunodeficient NOD/SCID mice. Two weeks following cell inoculation, the tumor-bearing mice then received repetitive intravenous injections of LNP-siASPM-v1 or an LNP-non-target control siRNA. (NT siRNA; 4 mg/kg every three days: total six doses). (B) shows the accumulation of the siRNA in the hepatic tumors of the treated mice. The tumors established in (A) were removed three days following two successive injections of LNP-siASPM-v1 (100 ag per mouse per injection) for cell dissociation and the cells were subjected to FACS analyses. Shown are representative FACS plots for Cy5 in the GFP-positive tumor cells isolated from LNP-formulated and Cy5-labeled siASPM-v1 or those from untreated tumors. (C) shows representative BLI of tumors at the indicated time following initiation of the treatments in (A). (D) shows the tumor bulk in (C) quantified as BLI normalized photon counts as a function of time. Data are shown as means±SEM (n=7 mice per group). *P≤0.05 compared to LNP-NT siRNA. (E) shows the percent survival as a function of time in mice described in (D). Arrows: siRNA injections.

FIGS. 21 (A) and (B) include several panels relating to the gene-silencing efficacy of unmodified and chemically modified siASPM-v1.7636 and siASPM-v1.4822 in cancer cells. (A) shows the transcript level of ASPM variant 1 (ASPM-v1) in breast cancer MDA-MB-436 cells transduced with siASPM-v1.7636, siASPM-v1.4822, or their 1:1 mixture, with or without chemical modifications as described in Table 5 analyzed using qRT-PCR analysis (mean±SEM; n=3). (B) shows the transcript level of ASPM-v1 in hepatocellular carcinoma HuH-1 cells transduced with unmodified or chemically modified siASPM-v1.7636, siASPM-v1.4822, or their 1:1 mixture as described in (A) (mean±SEM; n=3). ***P<0.001 versus chemically modified non-target (NT) siRNA (NT siRNA).

FIGS. 22 (A) and (B) include several panels relating to the effect of siASPM-v1 active pharmaceutical ingredient (API) on the invadopodia formation of cancer cells. (A) shows the inhibitory effect the siASPM-v1 API on the invadopodia formation of HCC HuH-1 cells. Shown on left are confocal views of the cortactin “F-actin” puncta (yellow), which represent the cross-sections of the downwardly protruding invadopodia, in HuH-1 cells transduced with siASPM-v1 API or a chemically modified non-target control siRNA (NT siRNA; both at 100 nM×72 hours) using the Lipofectamine LTX reagent. Right, the number of cortactin “F-actin” (representing invadopodia) per cell. Scale bar, 10 μm. Data are mean±SEM. ***P<0.001. (B) shows the inhibitory effect of siASPM-v1 API on the invadopodia formation of TNBC MDA-MB-436 cells. Shown on left are confocal views of the cortactin “F-actin” puncta (yellow), which represent the cross-sections of the downwardly protruding invadopodia, in HuH-1 cells transduced with siASPM-v1 API or non-target control siRNA (NT siRNA; both 100 nM×72 hours) using the Lipofectamine LTX reagent. Right, the number of invadopodia per cell. Scale bar, 10 μm. Data are mean±SEM. ***P<0.001.

FIG. 23 shows that the transduction with siASPM-v1 active pharmaceutical ingredient (API) inhibited the invasive capacities of hepatocellular carcinoma HuH-1 or breast cancer MDA-MB-436 cells. Shown on left are representative immunofluorescence images of the invaded cells, with cell nuclei stained with CYTOX-green (green). Scale bars=100 μm. Right, the numbers of invaded cells. Data are mean #SEM. ***P<0.001.

FIGS. 24 (A) and (B) include several panels relating to the effect of siASPM-v1 active pharmaceutical ingredient (API) on the Wnt, Hedgehog, and Notch pathway activities in cancer cells. HuH-1 hepatocarcinoma cells (A) or MDA-MB-436 breast cancer cells (B) carrying a triple luciferase reporter for the measurement of the Wnt, Hh, and Notch pathway activities were transduced with siASPM-v1 API or non-target control siRNA (NT siRNA) at 100 nM using Lipofectamine LTX Reagent for 48 hours. The cells were then stimulated with SHH (3 μg/ml×24 hours), WNT3A (250 ng/ml×16 hours), JAG1-Fc (5 μg/ml×24 hours), respectively, or vehicle for 24 hours before the measurement of the respective reporter activity. **P<0.01: ***P<0.001 compared to control.

FIG. 25 (A) and (B) include several panels relating to the gene-silencing efficacy of unmodified and chemically modified siASPM-v1.4360 and siASPM-v1.4822 in cancer cells. (A) shows the transcript level of ASPM-v1 in HuH-1 hepatocarcinoma cells transduced with 100 nM (for 48 hours) of siASPM-v1.4360 and siASPM-v1.4822, or their 1:1 mixture (designated as “siASPM-v1 active pharmaceutical ingredient version 2” or “siASPM-v1 API_V2”), or their chemically modified version analyzed using qRT-PCR analysis (mean±SEM; n=3). siNT, non-target siRNA. (B) shows the transcript level of ASPM-v1 in HCT-116 colorectal cancer cells transduced with unmodified or chemically modified siRNAs as in (A) (mean±SEM; n=3). ***P<0.001 compared to siNT.

FIG. 26 (A) and (B) include several panels relating to the inhibitory effect of siASPM-v1 active pharmaceutical ingredient version 2 (API_V2) on the invadopodia formation of cancer cells. (A) shows the inhibitory effect the siASPM-v1 API_V2 on the invadopodia formation of HuH-1 hepatocarcinoma cells. Shown on left are confocal views of the TKS5⁺Col1-3/4C⁺ puncta (yellow), which represent the cross-sections of the downwardly protruding invadopodia, in HuH-1 cells transduced with siASPM-v1 API_V2 or a chemically modified non-target control siRNA (m-siNT; both at 100 nM×72 hours). Right, the number of TKS5⁺ Col1-3/4C⁺ (representing functional invadopodia) per cell. Scale bar, 10 μm. Data are mean±SEM. ***P<0.001. (B) shows the inhibitory effect of siASPM-v1 API_V2 on the invadopodia formation of HCT-116 colorectal cancer cells. Shown on the left are confocal views of the TKS5⁺Col1-3/4C⁺ puncta (yellow), which represent the cross-sections of the downwardly protruding invadopodia, in HCT-116 cells transduced with siASPM-v1 API_V2 or m-siNT (both 100 nM×72 hours). Right, the number of invadopodia per cell. Scale bar, 10 μm. Data are mean±SEM. ***P<0.001.

FIG. 27 (A) and (B) include several panels relating to the inhibitory effect of siASPM-v1 active pharmaceutical ingredient version 2 (API_V2) on the invasive capability of cancer cells. (A) shows representative immunofluorescence images of the invaded HhH-1 hepatocellular carcinoma cells or HCT-116 colorectal cancer cells treated with chemically modified non-target siRNA (m-siNT) or siASPM-v1 API_V2, with cell nuclei stained with CYTOX-green (green). Scale bars=100 μm. (B) shows the number of invaded cells. Data are mean±SEM. ***P<0.001.

FIG. 28 shows the inhibitory effect of siASPM-v1 active pharmaceutical ingredient version 2 (API_V2) on the Wnt, Hedgehog (Hh), and Notch pathways, and TEAD activities in cancer cells. HuH-1 hepatocellular carcinoma cells (left) or HCT-116 colorectal cells (right) were lentivirally infected with the reporter constructs for the Wnt, Hh, Notch signaling pathways or the TEAD transcriptional activity. The cells were then transduced with chemically modified non-target siRNA (m-siNT) or siASPM-v1 API_V2 at 100 nM using Lipofectamine LTX Reagent for 48 hours, after which the cells were stimulated with SHH (3 μg/ml×24 hours), WNT3A (250 ng/ml×16 hours), JAG-Fc (5 ng/ml×24 hours), respectively, for 24 hours. **P<0.01; ***P<0.001 compared to m-siNT.

FIG. 29 (A) and (B) include several panels relating to the inhibitory effect of siASPM-v1 active pharmaceutical ingredient version 2 (API_V2) on the tumorsphere-forming capability of cancer cells. (A) shows representative phase contrast images of tumorspheres formed by HuH-1 hepatocellular carcinoma or HCT-116 colorectal cancer cells transduced with chemically modified non-target siRNA (m-siNT) or siASPM-v1 API_V2. Scale bar, 100 mm. (B) shows limiting dilution assay demonstrating the tumorsphere-forming efficacy (1/n) of HuH-1 or HCT-116 cells transduced with m-siNT or siASPM-v1 APL V2. n=8 independent experiments. Maximum likelihood estimates with a 95% confidence interval. ***P<0.001; the likelihood ratio test and chi-square test.

### DETAILED DESCRIPTION OF THE INVENTION

Unless stated otherwise, the following terms and phrases have the meanings provided below.

The term “ribonucleotide” and the phrase “ribonucleic acid” (RNA), refer to a modified or unmodified nucleotide or polynucleotide comprising at least one ribonucleotide unit. A ribonucleotide unit comprises an hydroxyl group attached to the 2′ position of a ribosyl moiety that has a nitrogenous base attached in N-glycosidic linkage at the 1′ position of a ribosyl moiety, and a moiety that either allows for linkage to another nucleotide or precludes linkage.

As used herein, the term “interfering RNA” or “RNAi” or “interfering RNA sequence” refers to double-stranded RNA (i.e., duplex RNA) that targets (i.e., silences, reduces, or inhibits) expression of a target gene (i.e., by mediating the degradation of mRNAs which are complementary to the sequence of the interfering RNA) when the interfering RNA is in the same cell as the target gene. Interfering RNA thus refers to the double-stranded RNA formed by two complementary strands or by a single, self-complementary strand. Specifically, RNAi molecule refers to shRNA, siRNA or dsRNA as disclosed herein. Small hairpin RNA (shRNA) is an RNA sequence that forms a rigid hairpin turn that can be used to silence gene expression by RNA interference. shRNA can be delivered to target cells using DNA plasmids, viral vectors or bacterial vectors. Double-stranded RNA (dsRNA) comprises a broad group of viruses. Small interfering RNA (siRNA) is a class of double-stranded RNA molecules, which includes duplexes of two separate strands, as well as single strands that can form hairpin structures comprising a duplex region. siRNAs are short (generally about 18-30 base pairs in length). siRNA can be used to silence gene expression by RNA interference. Furthermore, siRNAs can vary in length and contain varying degrees of complementarity to their target mRNA in the antisense strand. Some, but not all, siRNA have unpaired overhanging bases on the 5′ or 3′ end of the sense strand and/or the antisense strand.

As used herein, the term “complementary nucleotide sequence” refers to complementary RNA that is complementary to a region of the mRNA transcript of the target mutant gene (i.e., the “corresponding nucleotide sequence” of the target gene).

As used herein, an excipient is an inactive ingredient in a pharmaceutical composition. Examples of excipients include fillers or diluents, surfactants, binders, glidants, lubricants, disintegrants, and the like.

As used herein, the term “substantial identity” refers to a sequence that hybridizes to a reference sequence under stringent conditions, or to a sequence that has a specified percent identity over a specified region of a reference sequence.

As used herein, the phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assay” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization.

As used herein, the terms “substantially identical” or “substantial identity,” in the context of two or more nucleic acids, refer to two or more sequences or subsequences that are the same or have a specified percentage of nucleotides that are the same (i.e., at least about 60%, preferably 65%, 70%, 75%, preferably 80%, 85%, 90%, or 95% identity over a specified region), when compared and aligned for maximum correspondence ever a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 5, 10, 15, 20, 25, 30, 35, 40, 45, 50, 75, or 100 nucleotides in length.

The term “transfection” refers to a process by which agents are introduced into a cell.

The phrase “inhibiting expression of a target gene” refers to the ability of an siRNA molecule of the present invention to silence, reduce, or inhibit expression of a target gene. To examine the extent of gene silencing, a test sample (e.g., a biological sample from an organism of interest expressing the target gene or a sample of cells in culture expressing the target gene) is contacted with an siRNA that silences, reduces, or inhibits expression of the target gene. Expression of the target gene in the test sample is compared to expression of the target gene in a control sample that is not contacted with the siRNA. Control samples are assigned a value of 100%. Silencing, inhibition, or reduction of expression of a target gene is achieved when the value of test the test sample relative to the control sample is about 95%, 90%, 85%, 80%, 75%, 70%, 65%, 60%, 55%, 50%, 45%, 40%, 35%, 30%, 25%, 20%, or 10%, Suitable assays include, e.g., examination of protein or mRNA levels using techniques known to those of skill in the art such as dot blots, Northern blots, in situ hybridization, ELISA, immunoprecipitation, enzyme function, as well as phenotypic assays known to those of skill in the art.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventively treating” and “preventing” are used interchangeably and comprise a treatment of patients at risk to develop a condition mentioned hereinbefore, thus reducing said risk.

ASPM has been identified as a critical regulator of Wnt signaling pathways. ASPM expression was found to be indispensable for cellular responsiveness to canonical Wnt ligands, such as Wnt-3a, in pancreatic and prostate cancer cells. Mechanistic studies revealed that ASPM interacts with upstream activators of β-catenin, including disheveled (Dvl)-2 or Dvl-3, and axin and protease-activated receptor-1 (PAR-1) and inhibits the proteasome-dependent degradation of the Dvl protein, thereby increasing the protein abundance level of β-catenin and augments canonical Wnt signaling that is important to its oncogenic effect. ASPM exhibits its oncogenic and Wnt-activating effects mainly through the protein stabilization of Dvl, together with the role of DVL in both canonical and noncanonical Wnt signaling, suggest that ASPM may also function as a noncanonical Wnt signaling activator in glandular cancer cells or CSCs. It was discovered that ASPM and its binding partner DVL is profoundly up-regulated in CSCs in various types of cancer cells, including pancreatic, breast, non-small-cell-lung, and prostate cancers and hepatocellular carcinoma (HCC).

It has been predicted that several putative splicing variants of the ASPM transcripts may exist in normal and malignant human tissues, which encode the protein isoforms consisting of 3477 (isoform 1), 1892 (isoform 2), 1389 (isoform 3), and 1062 amino acids (isoform 4), respectively (Kouprina, N., Pavlicek, A., Collins, N. K., Nakano, M., Noskov, V. N., Ohzeki, J., Mochida, G. H., Risinger, J. I., Goldsmith, P., Gunsior, M., et al. (2005). The microcephaly ASPM gene is expressed in proliferating tissues and encodes for a mitotic spindle protein. Hum Mol Genet 14, 2155-2165). The specific upregulation of ASPM isoform 1, together with its prognostic significance, makes this isoform an ideal and potentially safe therapeutic target in cancer.

In one aspect, the present provides a polynucleotide, comprising a nucleotide sequence complimentary to the mRNA of the ASPM gene with a nucleotide sequence shown in SEQ ID NO: 1: b) a nucleotide sequence comprising a contiguous segment having at least 70%, at least 80%, or at least 90% sequence identity to the polynucleotide set forth in a).

The polynucleotide of the present disclosure can be a RNAi molecule such as shRNA, siRNA, miRNA, dsRNA or antisense oligonucleotide (ASO), or any derivatives thereof, that is complimentary to the coding or the noncoding region of the mRNA of the ASPM gene (SEQ ID NO:1) and thereby induces the specific degradation or reduce the amount thereof.

The polynucleotide of the present disclosure also can be complimentary to the mRNA encoded by the exon 18 of the human ASPM gene shown in SEQ ID NO:3, such that said polynucleotide only induces the degradation or reduces the amount of the transcript variant 1 of ASPM but not that of the other transcript variants.

Particularly, the polynucleotide of the present disclosure is a siRNA of about 15-60, 15-50, 15-50, or 15-40 (duplex) nucleotides in length, more typically about 15-30 or 15-25 (duplex) nucleotides in length, and is preferably about 20-24, 21-22, or 21-23 (duplex) nucleotides in length (e.g., each complementary sequence of the double-stranded siRNA is about 15-60, 15-50, 15-50, 15-40, 15-30, or 15-25 nucleotides in length, preferably about 20-24, 21-22, or 21-23 nucleotides in length, and the double-stranded siRNA is about 15-60, 15-50, 15-50, 15-40, 15-30, 15-25, or 19-25 base pairs in length, preferably about 20-24, 21-22, or 21-23 base pairs in length). siRNA duplexes may comprise 3′ overhangs of about 1 to about 4 nucleotides, preferably of about 2 to about 3 nucleotides and 5′ phosphate termini. Examples of siRNA include, without limitation, a double-stranded polynucleotide molecule assembled from two separate oligonucleotides, wherein one strand is the sense strand and the other is the complementary antisense strand; a double-stranded polynucleotide molecule assembled from a single oligonucleotide, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

In one embodiment of the present disclosure, the siRNA can be double stranded and can comprise at least one blunt end. For example, the siRNA can be an siRNA wherein both ends are blunt-ended; an siRNA wherein one end is blunt-ended and the other end comprise a 5′ 2 nucleotide overhang; an siRNA wherein one end is blunt-ended and the other end comprises a 3′ 2 nucleotide overhang; and/or a combination thereof. Alternatively, in this embodiment, the siRNA that is introduced into the cell can be double stranded and comprise a 5′ 2 nucleotide overhang at each end. In a particular embodiment, an overhang can comprise from about 1 nucleotide to about 5 nucleotides. In another embodiment, the siRNA can be double stranded and comprise at least 2 overhangs. An overhang can comprise from about 1 nucleotide to about 5 nucleotides. In a particular embodiment, each of the at least 2 overhangs comprise 2 nucleotides. In this embodiment of the invention, the siRNA can be an siRNA wherein both 3′ ends comprise a 2 nucleotide overhang; an siRNA wherein one end comprises a 3′ 2 nucleotide overhang and the other end comprises a 5′ 2 nucleotide overhang; and/or a combination thereof.

The siRNA may be modified to contain backbone residues or linkages which are synthetic, naturally occurring, and non-naturally occurring to form analogs, which have similar binding properties as the reference nucleic acid, and which are metabolized in a manner similar to the reference nucleotides. The siRNA may be modified according to process known in general knowledge and the modification includes replacement or addition of one or more atoms or groups in one or more nucleotide bases. Some examples of types of modifications that can comprise nucleotides that are modified with respect to the base moieties include but are not limited to, alkylated, halogenated, thiolated, aminated, amidated, or acetylated bases, individually or in combination. Certain examples include, for example, 2-propyladenine, 2-propylguanine, 2-aminoadenine, 1-methylinosine, 3-methyluridine, 5-propynyluridine, 5-propynylcytidine, 6-methyladenine, 6-methylguanine, N,N-dimethyladenine, 5-methylcytidine, 5-methyluridine and other nucleotides having a modification at the 5 position, 1-methyladenosine, 2-methyladenosine, 3-methylcytidine, 5-(2-amino) propyl uridine, 5-halocytidine, 5-halouridine, 4-acetylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, S-substituted uracils and thymines, azapyrimidines, carboxyhydroxyalkyl nucleotides, carboxyalkylaminoalkyl nucleotides, and alkylcarbonylalkylated nucleotides. Further specific examples of such analogs include, without limitation, phosphorothioates, phosphoramidates, methyl phosphonates, chiral-methyl phosphonates, 2′-O-methyl ribonucleotides, and peptide-nucleic acids (PNAs). Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Chemical modification of the siRNA comprises attaching a conjugate to the siRNA molecule. The conjugate can be attached at the 5′ and/or 3′-end of the sense and/or antisense strand of the siRNA via a covalent attachment such as, e.g., a biodegradable linker. The conjugate can also be attached to the siRNA, e.g., through a carbamate group or other linking group. In certain instances, the conjugate is a molecule that facilitates the delivery of the siRNA into a cell. Examples of conjugate molecules suitable for attachment to an siRNA include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof.

The siRNA can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA.

Substantial identity refers to a sequence that hybridizes to a reference sequence under stringent conditions, or to a sequence that has a specified percent identity over a specified region of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well-known in the art. Optimal alignment of sequences for comparison can be conducted.

A preferred example of algorithms that are suitable for determining percent sequence identity and sequence similarity are the BLAST and BLAST 2.0 algorithms. BLAST and BLAST 2.0 are used with the parameters described herein to determine percent sequence identity for the nucleic acids and proteins of the present invention. Software for performing BLAST analyses is publicly available through the National Center for Biotechnology Information (http://www.nebi.nlm.nih.gov/).

Once a potential siRNA sequence has been identified, the sequence can be analyzed using a variety of criteria known in the art. One of skill in the art will appreciate that sequences with one or more of the foregoing characteristics may be selected for further analysis and testing as potential siRNA sequences. siRNA sequences complementary to the siRNA target sites may also be designed.

The polynucleotide of the present disclosure is operatively linked to a control sequence or gene promoters that direct the expression of said polynucleotide in a particular tissue and/or type of cell. Such control sequences are well known within the art and can be constructed by any of a variety of manners known to those of skill.

Methods suitable for use in the disclosure to determine the expression of the ASPM mRNA comprise an assay selected from the group consisting of Northern blotting, reverse transcription polymerase chain reaction (PCR), RNase protection assay, cDNA or oligonucleotide microarray analysis, nucleotide sequencing, or the probe-based digital mRNA profiling technology such as the NanoString nCounter gene expression system (Geiss, G. K., Bumgarner, R. E., Birditt, B., Dahl, T., Dowidar, N., Dunaway, D. L., Fell, H. P., Ferree, S., George, R. D., Grogan, T., et al. (2008). Direct multiplexed measurement of gene expression with color-coded probe pairs. Nat Biotechnol 26, 317-325).

Methods suitable for use in the present disclosure to determine the expression of the ASPM protein comprise an assay selected from the group consisting of Western blotting, ELISA, immunoprecipitation, glutathione-S-transferase fusion protein pull-down, fluorescence anisotropy, fluorescence polarization, fluorescence resonance energy transfer, analytical ultracentrifugation, surface plasmon resonance, and isothermal titration calorimetry.

In one embodiment, the present disclosure provides a pool or kit of at least one siRNAs, preferably in the form of a kit or therapeutic reagent, wherein one strand of each of the siRNAs, the sense strand comprises a sequence that is substantially similar to a sequence within a target mRNA. The opposite strand, the antisense strand, will preferably comprise a sequence that is substantially complementary to that of the target mRNA. More preferably, one strand of each siRNA will comprise a sequence that is identical to a sequence that is contained in the target mRNA. Most preferably, each siRNA will be 15-25, 15-20 or 15-18 base pairs in length, and one strand of each of the siRNAs will be 100% complementary to a portion of the target mRNA. By increasing the number of siRNAs directed to a particular target using a pool or kit, one is able both to increase the likelihood that at least one siRNA with satisfactory functionality will be included, as well as to benefit from additive or synergistic effects. Further, when two or more siRNAs directed against a single gene do not have satisfactory levels of functionality alone, if combined, they may satisfactorily promote degradation of the target messenger RNA and successfully inhibit translation.

The siRNA duplexes within the aforementioned pools or kit of siRNAs may correspond to overlapping sequences within a particular mRNA, or non-overlapping sequences of the mRNA. However, preferably they correspond to non-overlapping sequences. Moreover, each siRNA may be selected randomly, or one or more of the siRNA may be selected according to the criteria discussed above for maximizing the effectiveness of siRNA

In an embodiment of the present disclosure, an anti-tumor reagent or composition can be delivered to a cell, a malignant tumor or an individual by direct transfection or transfection and expression via an expression vector. Appropriate expression vectors include mammalian expression vectors and viral vectors, into which has been cloned a polynucleotide encoding a sensitizing reagent with the appropriate regulatory sequences including a promoter to result in expression of said sensitizing reagent in a cell or a malignant tumor. Suitable promoters can be constitutive or development-specific promoters. Transfection delivery can be achieved by liposomal transfection reagents, known in the art (e.g., Xtreme transfection reagent, Roche, Alameda, CA; Lipofectamine formulations, Invitrogen, Carlsbad, CA). Delivery mediated by cationic liposomes and direct delivery are efficient. Another possible delivery mode is targeting using antibody to cell surface markers for the target cells.

For transfection, a composition comprising one or more nucleic acid molecules (within or without vectors) can comprise a delivery vehicle, including liposomes, for administration to a subject, carriers and diluents and their salts, and/or can be present in pharmaceutically acceptable formulations. Delivery of siRNA molecules is also described in several U.S. Patent Publications, including for example, 2006/0019912; 2006/0014289; 2005/0239687; 2005/0222064, and 2004/0204377, the disclosures of each of which are hereby incorporated herein by reference. Nucleic acid molecules can be administered to cells by a variety of methods known to those of skill in the art, including, but not restricted to, encapsulation in liposomes, by iontophoresis, by electroporation, or by incorporation into other vehicles, including biodegradable polymers, hydrogels, cyclodextrins (see, for example Gonzalez et al., 1999, Bioconjugate Chem., 10, 1068-1074; Wang et al., International PCT publication Nos. WO 03/47518 and WO 03/46185), poly(lactic-co-glycolic) acid (PLGA) and PLCA microspheres (see for example U.S. Pat. No. 6,447,796 and US Patent Application Publication No. 2002/130430), biodegradable nanocapsules, and bioadhesive microspheres, or by proteinaceous vectors (O'Hare and Normand, International PCT Publication No. WO 00/53722). In another embodiment, the nucleic acid molecules of the present disclosure can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

Examples of liposomal transfection reagents of use with this invention include, for example: CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); Cytofectin GSV, 2:1 (M/M) liposome formulation of a cationic lipid and DOPE (Glen Research); DOTAP (N-[1-(2,3-dioleoyloxy)-N,N,N-tri-methyl-ammoniummethylsulfate) (Boehringer Manheim); Lipofectamine, 3:1 (M/M) liposome formulation of the polycationic lipid DOSPA and the neutral lipid DOPE (GIBCO BRL); and(S) siPORT (Ambion); HiPerfect (Qiagen); X-treme GENE (Roche); RNAicarrier (Epoch Biolabs) and TransPass (New England Biolabs).

Many types of human solid cancers, such as breast cancer, non-small-cell lung carcinoma (NSCLC), PDAC, the scirrhous subtype of gastric adenocarcinoma, and the “stem/serrated/mesenchymal (SSM)” molecular subtype of colorectal cancer (CRC), are characterized by a pronounced stromal reaction termed “the desmoplastic response” (Isella, C., Terrasi, A., Bellomo, S. E., Petti, C., Galatola, G., Muratore, A., Mellano, A., Senetta, R., Cassenti, A., Sonetto, C., et al. (2015). Stromal contribution to the colorectal cancer transcriptome. Nat Genet 47, 312-319), which constitutes a major obstacle for the efficient transport of cancer therapeutics into the tumor. Recently, two nanoparticle-formulated chemotherapy, including albumin-bound paclitaxel (nab-paclitaxel) and liposome-encapsulated irinotecan, have been shown to extend the survival of patients with advanced PDAC. Both reagents could significantly increase the levels of the chemotherapeutic agents in the treated tumors (Wang-Gillam. A., Li, C. P., Bodoky, G., Dean, A., Shan, Y. S., Jameson, G., Macarulla, T., Lee, K. H., Cunningham, D, Blanc, J. F., et al. (2016). Nanoliposomal irinorecan with fluorouracil and folinic acid in metastatic pancreatic cancer after previous gemcitabine-based therapy (NAPOLI-I): a global, randomised, open-label, phase 3 trial. Lancet 387, 545-557), suggesting that nanoparticle formulation is a clinically validated approach to improve the treatment efficacy of desmoplastic cancers. In liver disease, parenterally administration of a lipid nanoparticle (LNP)-formulated siRNA specific for transthyretin (Patisiran, Alnylam Pharmaceuticals, MA, USA) has been shown to reduce up to 86.8% of transthyretin produced by the liver in patients with hereditary transthyretin-mediated amyloidosis and thus became the first clinically approved RNAi drug (Adams, D., Gonzalez-Duarte, A., O'Riordan, W. D., Yang, C. C., Veda, M., Kristen, A. V., Tournev, 1., Schmidt, H. H., Coelho, T., Berk, J. L., et al. (2018). Patisiran, an RNAi Therapeutic, for Hereditary Transthyretin Amyloidosis. N Engl J Med 379, 11-21). Moreover, nanoparticle-delivered siRNA therapy, such as cyclodextrin polymer-based nanoparticles carrying siRNA targeting ribonucleotide reductase M2 (RRM2) and lipid nanoparticles carrying siRNA targeting VEGF-A and kinesin spindle protein (KSP), have shown promising pharmacodynamics and tolerability and anti-tumor efficacy in some of the treated patients in phase 1 clinical trials. Moreover, the systemic delivery of siRNA targeting tumor-driving genes such as BCR-ABL has also showed significant therapeutic efficacy in a murine orthotopic model of hepatocellular carcinoma (HCC) (Tabernero, J., Shapiro, G. L., LoRusso, P. M., Cervantes, A., Schwartz, G. K., Weiss, G. J., Paz-Ares, T., Cho, D. C., Infante, J. R., Alsina, M., et al. (2013). First-in-humans trial of an RNA interference therapeutic targeting VEGF and KSP in cancer patients with liver involvement. Cancer Discov 3, 406-417). As opposed to microRNA, siRNA silences only one mRNA target and therefore its genetic and biological effect is highly specific and associated with less off-target effects as exemplified by the recent approval of the first-in-class liposomal siRNA targeting transthyretin in patients with hereditary amyloidosis (Adams, D., Gonzalez-Duarte, A., O'Riordan, W. D., Yang, C. C., Ueda, M., Kristen, A. V., Tournev, I., Schmidt, H. H., Coelho, T., Berk, J. L., et al. (2018). Patisiran, an RNAi Therapeutic, for Hereditary Transthyretin Amyloidosis, N Engl J Med 379, 11-21). In complex multigenic diseases such as human malignancies, the implementation of siRNA-based therapy requires the identification of “driver” genes that serve as the key regulators of the disease pathology.

Therapeutic gene delivery into tumor cells or malignant tissues can be achieved by using nonviral vehicles, such as lipid-based and polymeric materials). For instance, a tumor-targeting immunoliposome nanocomplex termed scL, in which the therapeutic molecule payload is encapsulated within a cationic liposome with its surface decorated with an anti-transferrin receptor (TfR) single-chain antibody fragment, has been designed to target tumor cells via the TfR highly expressed on their surface. A series of studies have demonstrated that the scL nanocomplex can specifically delivers various payloads, including plasmid DNA, siRNA, and small molecules, to both primary and metastatic tumor cells and even cancer stem cells both in vitro and in vivo. Systemic administration of the p53 plasmid DNA encapsulated with the scL immunoliposome nanoparticle termed TfRscFv-Lip p53 or “SGT-53” have been shown to induce tumor-specific expression of the exogenous p53 and thereby enhance the efficacy of chemo- and radio-therapy in various pre-clinical models of human tumors such as breast cancer, malignant glioma, head- and neck cancer, and pancreatic cancer. Importantly, the clinical applicability of the SGT-53 has been shown in a recent phase 1 clinical trial, which revealed well tolerance of the study participants at the therapeutic doses tested. Significantly, PCR analysis of the tumor tissues revealed the clear presence of the exogenous p53 transgene, lending a solid support to the scL nanoparticles as a valid systemic delivery vehicle to effectively deliver the therapeutic gene to human tumors.

A lipid nanoparticle (LNP) suitable for in vivo delivery of oligonucleotides such as siRNA to liver or tumor tissues has been generated with a rational-design approach by AlCana Technologies (Vancouver, BC) (Jayaraman. M., Ansell, S. M., Mui, B. L., Tam, Y. K., Chen, J., Du, X., Butter, D., Eltepu, L., Matsuda, S., Narayanannair, J. K., et al. (2012). Maximizing the potency of siRNA lipid nanoparticles for hepatic gene silencing in vivo. Angew Chem Int Ed Engl 51, 8529-8533). Said LNP formulation consists of four lipid components, including the ionizable cationic amino lipid DLin-MC3-DMA, which complexes with siRNA, the amphipathic phospholipid distearoyl-phophatidylcholine (DSPC), cholesterol and a coat lipid poly(ethylene glycol) lipid 1,2-dimyristoyl-rac-glycerol-methoxy (poly(ethylene glycol) (DMG-PEG) mixed at the molar ratio of 50:10:38.5:1.5. The particle size of the DLin-MC3-DMA LNP is in the range of 70-90 nm (Jayaraman, M., Ansell, S. M., Mui, B. L., Tam, Y. K., Chen, J., Du, X., Butler, D., Eltepu, L., Matsuda, S., Narayanannair, J. K., et al. (2012). Maximizing the potency of siRNA lipid nanoparticles for hepatic gene silencing in vivo. Angew Chem Int Ed Engl 51, 8529-8533), which is associated with an extended circulation times and permit its leakage into the tumor tissues through the leaky endothelial fenestrations (with estimated pore sizes of 380-780 mm), a phenomenon known as the “enhanced permeability and retention (EPR)” effect (Agarwal. R., and Roy, K. (2013). Intracellular delivery of polymeric nanocarriers: a matter of size, shape, charge, elasticity and surface composition. Ther Deliv 4, 705-723).

The DLin-MC3-DMA-based LNP formation has been used in the first FDA-approved siRNA drug patisiran (Onpattro) developed by Alnylam Pharmaceuticals. It has also been used for systemic delivery of siRNA targeting tumor-driving genes such as BCR-ABL, VEGF-A and kinesin spindle protein (KSP), which showed significant therapeutic efficacy in a mouse model of chronic myeloid leukemia (CML) or a murine orthotopic model of hepatocellular carcinoma (HCC). Systemic delivery of siRNA encapsulated using the DLin-MC3-DMA-based LNP has been shown to be safe and generally well-tolerated in a phase 1 clinical trial except some infusion-related reactions and transient proinflammatory cytokine induction (Tabernero, J., Shapiro, G. I, LoRusso, P. M., Cervantes, A., Schwartz, G. K. Weiss, G. J., Paz-Ares, L., Cho, D. C., Infante. J. R., Alsina, M., et al. (2013). First-in-humans trial of an RNA interference therapeutic targeting VEGF and KSP in cancer patients with liver involvement. Cancer Discov 3, 406-417).

Recently, a next-generation LNP-based delivery vehicle contains a highly biodegradable ionizable cationic lipid CL4H6, cholesterol, and 1,2-Dimirystoyl-sn-glycero, methoxyethyleneglycol 2000 ether (PEG-DMG2000) mixed at molar ratio of 60:40:1 has been described (Sato, Y., Hashiba, K., Sasaki, K., Maeki, M., Tokeshi, M., and Harashima, H. (2019). Understanding structure-activity relationships of pH-sensitive cationic lipids facilitates the rational identification of promising lipid nanoparticles for delivering siRNAs in vivo. J Control Release 295, 140-152). CL4H6 was systematically derivatized based on structure-activity relationship studies of the hydrophilic head group and hydrophobic tail of pH-sensitive cationic lipids and has a higher in vivo gene silencing activity than the benchmark lipid DLin-MC3-DMA (50% effective dose (ED50): 0.0025 mg/kg versus 0.005 mg/kg) in mouse factor VII models (Jayaraman, M., Ansell, S. M., Mui. B. L., Tam, Y. K., Chen, J., Du, X., Butler, D., Eltepu. L., Matsuda, S., Narayanannair, J. K., et al. (2012). Maximizing the potency of siRNA lipid nanoparticles for hepatic gene silencing in vivo. Angew Chemi Int Ed Engl 51, 8529-8533; Sato, Y., Hashiba, K., Sasaki, K., Maeki, M., Tokeshi, M., and Harashima, H. (2019). Understanding structure-activity relationships of pH-sensitive cationic lipids facilitates the rational identification of promising lipid nanoparticles for delivering siRNAs in vivo. J Control Release 295, 140-152). Whilst DLin-MC3-DMA is used in the first FDA-approved siRNA drug patisiran (Onpattro) developed by Alnylam Pharmaceuticals, it may cause liver toxicity and a significant weight loss when given to mice at a high dose range (>3 mg/kg) (Jayaraman, M., Ansell, S. M., Mui, B. L., Tam, Y. K., Chen, J., Du, K., Butler, D., Eltepu, L., Matsuda, S., Narayanannair, J. K., et al. (2012). Maximizing the potency of siRNA lipid nanoparticles for hepatic gene silencing in vivo. Angew Chem Int Ed Engl 51, 8529-8533). By contrast, CL4H6 contain biodegradable ester bonds in its hydrophobic tails and its level rapidly declines in the liver or spleen after 24 hours. The highly biodegradable nature of CL4H6 substantially reduces the likelihood of inducing liver or tissue toxicities especially in repetitive dosing schedules that are required in the treatment of cancers.

A siRNA or an oligonucleotide can be encapsulated by a LNP by mixing siRNA or oligonuclotides dissolved in 10 mM citrate buffer (pH 3.0) with a LNP in dissolved ethanol using a microfluidic instrument such as the NanoAssemblr™ system (Precision Nanosystems, Vancouver, BC, Canada) at a flow rate of 0.5 ml/min and a flow rate ratio of 1:3 (lipid:siRNA=0.125 ml/min: 0.375 ml/min). Syringe pumps (Harvard Apparatus, MA, USA) can be used to control the flow rate. The resulting LNP/siRNA mixture solution is then dialyzed against phosphate buffered saline using Spectra Por 4 dialysis membranes (Spectrum Laboratories, Rancho Dominguez, CA, USA). The LNP-encapsulated siRNA solution was then concentrated by ultrafiltration using an Amicon Ultra-15 unit (MWCO 50 kDa, Merch Millipore, Burlington, MA, USA). The size (number-weighted mean diameter) and ζ-potential of the LNPs were measured by a Zetasizer Nano ZS ZEN3600 instrument (Malvern Instruments, Worcestershire, UK). The encapsulation efficiency and total concentration of siRNA were measured using the Quanti-IT™ RiboGreen RNA Reagent and Kit (Invitrogen, Waltham, MA, USA).

Tumor-targeting of lipid-based nanoparticles can also be achieved by using a CXCR-4 antagonist AMD-3100 as a targeting moiety. CXCR-4 is upregulated after the targeted agent sorafenib treatment in hepatocellular carcinoma (HCC) and thereby the AMD-modified nanoparticles can efficient deliver VEGF siRNA into HCC, which synergizes with sorafenib to induce antiangiogenic effects and suppressing tumor growth and metastasis (Liu, J. Y., Chiang, T., Lin, C. H., Chern, G. G., Lin Ts, T, Gao, D. Y., and Chen, Y. (2015). Delivery of siRNA Using CXCR4-targeted Nanoparticles Modulates Tumor Microenvironment and Achieves a Potent Antitumor Response in Liver Cancer. Mol Ther 23, 1772-1782). Interestingly, even without specific tumor-targeting moieties, amphipathic liposomes or neutral lipid emulsions such as MaxSuppressor™ (BIOO Scientific, TX, USA) or SMARTICLES (Marina Biotech, WA, USA), were found to be able to efficiently deliver RNAi agents into tumors. Most lipid-based delivery systems contain cationic lipids and have a number of shortcomings that can be attributed to charge. It contrast, these neutral or amphoteric liposomes are neutral or anionic at neural and higher pH while are cationic at low pH. In biofluids with a pH of 7-7.5, the nanoparticles assume a slightly anionic character that may prevent unwanted interactions with the negative charge of cellular membranes in the endothelium and other tissues. For the same reasons, these liposomes may be less toxic than those containing cationic lipids. Since the pH tends to be lower in tumor areas, the particles may become cationic in these areas and adhere to tumor cells. The SMARTICLES formulation has been used to deliver tumor-suppressive miRNA, such as miR-34a and let-7, into tumors in animal models of HCC, prostate cancer and lung cancer, which led to a significant tumor regression and prolonged survival (Cortez, M. A., Valdecanas, D., Niknam, S., Peltier, H. J., Diao, L., Giri, U., Komaki, R., Calin, G. A., Gomez, D. R., Chang, J. Y., et al. (2015). In Vivo Delivery of miR-34a Sensitizes Lung Tumors to Radiation Through RAD51 Regulation. Mol Ther Nucleic Acids 4, e270).

Alternatively, using a combinatorial chemical synthesis, a low molecular weight polyamines and lipid compound, termed 7C1, was identified to deliver siRNA to lung endothelium without significantly transfecting immune cells or hepatocytes. In a KrasG12Dp53flox/flox transgenic lung adenocarcinoma model, treatment of tumor-bearing mice with a microRNA34 mimic and siRNA targeting the oncogene Kras using 7C1 nanoparticles reduced K-ras gene expression and MAPK signaling, inhibited tumor growth, synergized with chemotherapy to prolong survival.

The present disclosure also pertains to a pharmaceutical composition comprising an anti-tumor reagent as herein defined optionally in combination with a pharmaceutical carrier, diluent and/or adjuvant. Any suitable pharmaceutically acceptable diluent, adjuvant, carrier or excipient can be used in the present compositions (See e.g., Remington: The Science and Practice of Pharmacy, Alfonso R. Gennaro (Editor) Mack Publishing Company. April 1997). Preferred pharmaceutical forms would be in combination with sterile saline, dextrose solution, or buffered solution, or other pharmaceutically acceptable sterile fluids. Alternatively, a solid carrier, may be used such as, for example, microcarrier beads. Such compositions include the anti-tumor reagent in an effective amount, sufficient to provide a desired therapeutic or prophylactic effect, and a pharmaceutically acceptable carrier or excipient. An “effective amount” includes a therapeutically effective amount or a prophylactically effective amount.

For gene therapy vectors, the dosage to be administered may depend to a large extent on the condition and size of the subject being treated as well as the therapeutic formulation, frequency of treatment and the route of administration. Regimens for continuing therapy, including dose, formulation, and frequency may be guided by the initial response and clinical judgment. The parenteral route of injection into the blood vessel or interstitial space of tissue may be preferred, although other parenteral routes, such as inhalation of an aerosol formulation, may be required in specific administration. In some protocols, a formulation comprising the gene and gene delivery system in an aqueous carrier is injected into tissue in appropriate amounts.

### Sequence Listing

SEQ ID NO: 1 sets out the nucleotide sequence
of the human ASPM transcript, transcript variant
1 (NCBI Reference Sequence: NM_018136.4).
ATTGGTGGAGGCGGCAAGTTTAAACAGAGTCAAAACGCCATACTTGTTTG
GCTCCTCTTTTTAATTTGCGAGTTTATTGGGCTTGTTTTCTGTTTTCTAG
GGAGTAGGTTAGTGGAAAAGAAAAAGGGCCGAATTCACTCCCACGACCTC
TACAGCCCCCCCTGAGGGGAAGCGGTCAGCGTAAGTCCCGGATCCCCGCT
CCGGAGCCGCCTCGTGGGAGGGGGGCAAGGAGATCCAGGAGGGGTCTCGA
ATCTGCCATGGCGAACCGGCGAGTGGGGCGAGGCTGCTGGGAAGTGAGCC
CGACCGAGCGGAGGCCGCCCGCGGGGCTGCGGGGCCCCGCGGCCGAGGAG
GAGCCGTCTTCCCCGCCGGTCCTGTCTCTCAGCCACTTCTGCAGGTCTCC
TTTCCTTTGCTTCGGGGACGTTCTCCTGGGAGCCTCACGGACGCTGTCTC
TGGCCCTAGACAACCCTAACGAGGAGGTGGCAGAAGTGAAGATCTCCCAC
TTCCCGGCCGCGGACCTGGGCTTCAGTGTGTCGCAGCGCTGTTTCGTGTT
GCAGCCTAAAGAGAAAATTGTTATTTCTGTTAACTGGACACCACTCAAAG
AAGGCCGAGTAAGAGAGATTATGACATTTCTTGTAAATGATGTTCTGAAA
CACCAAGCTATATTACTAGGAAATGCAGAAGAGCAGAAAAAGAAAAAGAG
GAGTCTTTCGGATACCATTAAAAAGAAGAAAATTTCAGCCTCTACAAGTC
ACAACAGAAGGGTTTCAAATATTCAGAATGTTAATAAAACATTTAGTGTT
TCCCAAAAAGTTGACAGAGTTAGGAGCCCACTACAAGCTTGTGAAAACTT
GGCTATGAATGAAGGCGGTCCCCCAACAGAAAACAATTCTTTAATACTTG
AAGAAAATAAAATACCCATATCACCTATTAGCCCTGCTTTCAATGAATGC
CATGGTGCAACTTGCTTGCCACTCTCTGTACGTCGATCTACTACCTACTC
ATCTCTTCATGCATCAGAAAATAGGGAACTATTAAATGTACACAGTGCCA
ACGTTTCAAAAGTTTCTTTTAATGAGAAGCTGTAACTGAAACTTCCTTT
AATTCCGTAAATGTTAATGGCCAAAGAGGAGAGAATAGTAAACTTAGTCT
TACCCCCAACTGTTCTTCAACTTTGAACATTACACAAAGCCAAATACATT
TTCTAAGTCCAGATTCTTTTGTAAATAATAGTCATGGAGCTAATAATGAA
CTAGAATTAGTAACATGTCTTTCATCAGATATGTTTATGAAAGATAATTC
ACAGCCTGTGCATTTGGAATCAACAATTGCACATGAAATTTATCAGAAAA
TTTTAAGTCCAGATTCTTTCATAAAAGATAATTATGGACTAAATCAGGAT
CTAGAATCAGAGTCAGTTAATCCTATTTTATCCCCTAATCAATTTTTAAA
AGATAACATGGCATATATGTGTACATCTCAGCAAACATGTAAAGTACCAT
TATCAAATGAAAATTCTCAAGTCCCACAGTCTCCTGAAGATTGGAGAAAA
AGTGAAGTTTCGCCACGTATTCCTGAATGTCAGGGTTCAAAATCTCCCAA
AGCTATTTTTGAAGAACTAGTAGAAATGAAGTCAAATTACTACAGTTTTA
TAAAACAAAATAATCCTAAATTTTCTGCAGTTCAGGATATTTCTAGTCAT
AGCCACAATAAACAACCTAAGAGACGTCCAATACTTTCTGCCACTGTTAC
TAAAAGGAAGGCCACCTGTACCAGAGAAAACCAAACTGAGATTAATAAAC
CAAAAGCAAAAAGATGTCTCAACAGTGCAGTGGGTGAACATGAAAAAGTA
ATAAATAATCAAAAGGAAAAAGAAGATTTTCATTCTTATCTTCCAATTAT
AGATCCAATATTAAGTAAATCTAAGAGTTATAAAAACGAGGTAACACCCT
CTTCGACAACAGCTTCAGTTGCTCGGAAAAGAAAGAGCGATGGAAGCATG
GAAGATGCAAATGTGAGAGTTGCAATTACAGAACATACAGAAGTGCGAGA
AATCAAAAGAATCCATTTTTCTCCCTCAGAGCCTAAAACATCAGCTGTTA
AGAAAACAAAAAATGTGACAACACCCATCTCAAAACGTATTAGCAACAGA
GAGAAATTAAACCTGAAGAAGAAAACTGATTTATCAATATTCAGAACTCC
AATTTCTAAAACAAACAAAAGGACAAAACCCATTATCGCTGTGGCACAGT
CCAGTTTGACCTTCATAAAACCATTAAAAACAGATATTCCCAGACACCCG
ATGCCATTTGCTGCAAAAAACATGTTTTATGATGAACGCTGGAAGGAAAA
GCAGGAACAGGGCTTCACTTGGTGGTTAAATTTTATATTAACCCCTGATG
ACTTCACTGTAAAAACAAATATTTCTGAAGTAAATGCTGCTACTCTTCTT
TTGGGAATAGAGAATCAACATAAAATAAGTGTTCCTAGAGCACCTACAAA
AGAGGAAATGTCTCTCAGAGCTTATACTGCTCGGTGTAGGTTAAACAGAC
TACGTCGTGCAGCATGCCGTTTGTTTACTTCTGAAAAAATGCTTAAAGCT
ATTAAAAAGCTTGAAATTGAAATTGAAGCTAGGCGGTTAATTGTTCGAAA
AGATAGACACCTATGGAAAGATGTGGGAGAACGTCAGAAAGTCCTGAATT
GGCTGTTGTCCTACAATCCTTTGTGGCTTCGAATTGGTCTAGAGACAACT
TATGGAGAACTCATATCTTTGGAAGATAACAGTGATGTCACAGGGTTGGC
TATGTTTATTCTGAATCGCCTACTTTGGAATCCTGATATAGCAGCTGAGT
ATAGACACCCCACTCTTCCTCACCTGTATAGAGATGGTCATGAAGAAGCT
TTGTCCAAGTTTACATTGAAAAAGTTATTGTTGTTGGTCTGTTTTCTTGA
TTATGCTAAAATTTCCAGACTCATTGATCATGATCCTTGTCTCTTCTGTA
AAGATGCCGAATTCAAGGCTAGTAAAGAAATCCTTTTGGCTTTTTCACGA
GATTTCCTAAGTGGTGAAGGTGACCTTTCCCGTCACCTTGGCTTATTGGG
ATTACCTGTTAACCATGTTCAGACACCATTTGATGAATTTGATTTTGCCG
TTACAAATCTTGCCGTAGACTTGCAATGTGGAGTGCGCCTTGTGCGAACC
ATGGAACTTCTCACACAGAACTGGGACCTCTCAAAGAAACTCAGGATTCC
GGCAATAAGTCGTCTTCAAAAGATGCACAATGTTGACATTGTTCTTCAAG
TTCTTAAATCACGAGGAATTGAATTAAGTGATGAGCATGGAAATACAATT
CTATCTAAGGATATTGTGGATAGGCACAGAGAAAAAACTCTCAGGTTGCT
TTGGAAAATAGCGTTTGCTTTTCAGGTGGATATTTCCCTTAACTTAGATC
AATTAAAGGAAGAAATTGCCTTTCTAAAACACACAAAGAGTATAAAGAAA
ACAATATCTCTACTATCATCCCATTCTGATGATCTTATTAATAAGAAAAA
AGGCAAAAGGGATAGTGGTTCCTTTGAACAATATAGTGAAAACATAAAGT
TATTGATGGATTGGGTAAATGCTGTTTGTGCCTTCTATAATAAAAAGGTG
GAGAATTTTACAGTGTCTTTCTCAGACGGCCGTGTGTTATGTTACCTGAT
CCACCATTACCATCCTTGCTATGTGCCATTTGACGCTATATGTCAGCGTA
CTACTCAAACTGTGGAATGTACGCAAACTGGTTCAGTGGTATTAAATTCA
TCATCTGAATCTGATGACAGTTCTCTGGATATGTCTCTTAAAGCATTTGA
TCATGAAAATACTTCAGAGCTATACAAAGAGCTCCTAGAAAATGAAAAGA
AAAATTTTCACTTGGTTAGGTCTGCAGTTAGAGACCTTGGTGGAATACCT
GCTATGATTAATCATTCAGATATGTCAAATACAATTCCAGATGAAAAGGT
GGTTATTACCTATTTGTCATTTCTTTGTGCAAGGCTTTTGGATCTTCGTA
AAGAAATAAGAGCTGCTCGACTCATACAAACAACATGGAGAAAATATAAA
CTAAAAACAGATCTCAAACGCCATCAGGAGAGAGAGAAAGCTGCAAGAAT
TATTCAATTGGCTGTAATCAATTTTCTAGCAAAACAAAGATTGAGAAAAA
GAGTTAATGCAGCACTCGTCATTCAGAAATATTGGCGAAGAGTCTTAGCA
CAGAGAAAATTATTAATGTTAAAAAAGGAAAAGCTGGAAAAAGTTCAAAA
TAAAGCAGCATCACTTATTCAGGGATATTGGAGAAGATATTCCACTAGAC
AAAGATTTCTGAAATTGAAATATTATTCAATCATCCTGCAATCTAGGATA
AGAATGATAATTGCTGTTACATCTTATAAACGATATCTTTGGGCTACAGT
TACAATTCAGAGGCATTGGCGTGCTTATTTAAGAAGAAAACAAGATCAAC
AAAGATATGAAATGCTAAAATCATCAACTCTTATAATCCAATCTATGTTC
AGAAAATGGAAGCAACGTAAAATGCAATCACAAGTAAAAGCTACAGTAAT
ATTGCAAAGAGCTTTTAGAGAATGGCATTTAAGAAAACAAGCTAAAGAAG
AAAATTCTGCTATTATCATACAATCATGGTATAGAATGCATAAAGAATTA
CGGAAATATATTTATATTAGATCTTGTGTTGTTATCATTCAGAAAAGATT
TCGGTGCTTTCAAGCCCAAAAGTTATATAAAAGAAGAAAAGAGTCCATAC
TAACCATCCAGAAGTACTACAAAGCATATCTGAAAGGAAAGATTGAGCGC
ACCAACTATTTGCAGAAACGAGCTGCAGCCATTCAATTACAAGCTGCTTT
TAGGAGACTGAAAGCTCATAATTTATGTAGACAAATTAGAGCTGCTTGTG
TTATTCAGTCATACTGGAGAATGAGACAAGACAGAGTTCGATTTTTAAAC
CTTAAGAAGACTATTATCAAATTTCAGGCACATGTAAGAAAACATCAACA
ACGACAGAAATATAAGAAGATGAAGAAAGCAGCTGTTATAATTCAGACTC
ATTTCCGAGCTTATATTTTTGCCATGAAAGTTCTAGCATCTTACCAGAAA
ACACGCTCTGCTGTCATTGTGCTGCAGTCTGCATATAGAGGGATGCAAGC
CAGGAAAATCTATATTCACATCCTCACATCTGTTATAAAGATTCAATCAT
ATTATCGTGCTTATGTTTCTAAAAAGGAATTTTTGAGCCTAAAAAATGCT
ACAATAAAATTGCAGTCAACTGTTAAGATGAAACAAACACGTAAACAATA
TTTGCATTTAAGAGCAGCTGCACTATTTATCCAGCAATGTTACCGTTCCA
AAAAAATAGCTGCACAAAAGAGAGAAGAGTATATGCAGATGCGGGAATCT
TGTATCAAACTGCAAGCATTTGTTAGAGGATACCTTGTCCGAAAGCAGAT
GAGGTTACAAAGAAAAGCTGTTATTTCACTACAGTCTTATTTCAGAATGA
GAAAGGCTCGGCAGTATTATCTGAAAATGTATAAAGCAATTATTGTCATT
CAGAATTACTATCATGCATACAAAGCACAGGTCAATCAGAGGAAGAACTT
CTTGCAAGTCAAAAAAGCAGCTACTTGCTTGCAAGCAGCTTACAGAGGTT
ATAAAGTACGCCAGCTAATCAAACAACAATCTATAGCTGCTCTTAAAATT
CAGTCTGCTTTTAGAGGCTATAATAAAAGGGTAAAATATCAATCTGTGCT
TCAATCTATAATAAAGATTCAGAGATGGTACAGGGCGTACAAGACTCTTC
ATGATACAAGAACACATTTTTTGAAGACAAAGGCAGCTGTGATTTCCCTC
CAGTCTGCTTATCGTGGCTGGAAGGTTCGGAAACAGATTAGAAGGGAACA
TCAAGCTGCCTTGAAGATTCAGTCTGCTTTTAGAATGGCCAAGGCCCAGA
AACAGTTTAGATTGTTTAAAACAGCAGCATTAGTCATCCAGCAAAATTTC
AGAGCATGGACTGCAGGAAGGAAGCAATGTATGGAGTATATTGAACTCCG
TCATGCGGTACTGGTGCTTCAATCTATGTGGAAGGGAAAAACACTGAGAA
GACAGCTTCAAAGGCAACATAAATGTGCTATCATCATACAGTCATACTAT
AGAATGCATGTGCAACAAAAGAAGTGGAAAATCATGAAAAAAGCTGCTCT
TCTGATTCAAAAGTATTATAGGGCTTACAGTATTGGAAGAGAACAGAATC
ATTTATATTTGAAAACAAAAGCAGCTGTAGTAACTTTACAGTCAGCTTAT
CGTGGTATGAAAGTGAGAAAAAGAATAAAGGATTGCAACAAAGCAGCAGT
CACTATACAGTCTAAATACAGAGCTTACAAAACCAAAAAGAAATATGCAA
CCTATAGAGCTTCAGCTATTATAATTCAGAGATGGTATCGAGGTATTAAA
ATTACAAACCATCAGCATAAGGAGTATCTTAATTTGAAGAAGACAGCAAT
TAAAATCCAATCTGTTTATAGAGGTATTAGAGTTAGAAGACATATTCAAC
ACATGCACAGGGCAGCCACTTTTATTAAAGCCATGTTTAAAATGCATCAG
TCAAGAATAAGTTACCATACAATGAGAAAAGCAGCTATTGTTATTCAAGT
AAGATGTAGAGCATATTATCAAGGTAAAATGCAGCGTGAAAAGTACCTGA
CAATTTTGAAAGCTGTTAAAGTCCTTCAGGCAAGTTTTAGAGGAGTAAGA
GTTAGACGGACTCTTAGAAAGATGCAGACTGCAGCAACACTCATTCAGTC
AAACTACAGAAGATACAGACAGCAAACATACTTTAATAAGTTAAAGAAAA
TAACAAAAACAGTACAGCAAAGATACTGGGCAATGAAAGAAAGAAACATA
CAATTTCAAAGGTATAACAAACTGAGGCATTCTGTAATATACATTCAGGC
TATTTTTAGGGGAAAGAAAGCTAGAAGACATTTAAAAATGATGCATATAG
CCGCAACTCTCATTCAGAGGAGATTTAGAACTCTAATGATGAGAAGAAGA
TTCCTCTCTCTCAAGAAAACTGCTATTTTGATTCAGAGAAAATATCGGGC
ACATCTTTGTACAAAGCATCACTTACAGTTCCTTCAGGTACAAAATGCAG
TTATTAAAATCCAGTCATCATACAGAAGATGGATGATAAGGAAAAGGATG
CGAGAGATCCACAGGGCTGCTACTTTCATCCAGTCTACTTTCAGAATGCA
CAGATTACATATGAGATATCAGGCTTTGAAACAGGCCTCCGTTGTGATCC
AACAGCAATACCAAGCAAATAGAGCTGCAAAACTGCAGAGGCAGCATTAT
CTCAGACAAAGACACTCTGCTGTGATCCTTCAGGCTGCATTCAGGGGTAT
GAAAACTAGAAGACATTTGAAGAGTATGCATTCCTCTGCAACCCTTATTC
AGAGTAGGTTTAGATCATTACTGGTGAGGAGAAGATTCATTTCCCTCAAA
AAAGCTACTATTTTTGTTCAGAGGAAATATCGAGCCACCATTTGTGCCAA
ACATAAATTGTACCAATTCTTGCACTTAAGAAAGGCAGCCATTACAATAC
AGTCATCTTACAGAAGACTGATGGTAAAGAAGAAGTTACAAGAAATGCAA
AGGGCTGCAGTTCTCATTCAGGCTACTTTCAGGATGTACAGAACATATAT
TACATTTCAGACTTGGAAACATGCTTCAATTCTAATTCAGCAACATTATC
GAACATATAGAGCTGCAAAATTACAAAGAGAAAATTATATCAGACAATGG
CATTCTGCTGTGGTTATTCAGGCTGCATATAAAGGAATGAAAGCAAGACA
ACTTTTAAGGGAAAAACACAAAGCTTCTATCGTAATACAAAGCACCTACA
GAATGTATAGGCAGTATTGTTTCTACCAAAAGCTTCAGTGGGCTACAAAA
ATCATACAAGAAAAATATAGAGCAAATAAAAAGAAACAGAAAGTATTTCA
ACACAATGAACTTAAGAAAGAGACTTGTGTTCAGGCAGGTTTTCAGGACA
TGAACATAAAAAAACAGATTCAGGAACAGCACCAGGCTGCCATTATTATT
CAGAAGCATTGTAAAGCCTTTAAAATAAGGAAGCATTATCTCCACCTTAG
AGCAACAGTAGTTTCTATTCAAAGAAGATACAGAAAACTAACTGCAGTGC
GTACCCAAGCAGTTATTTGTATACAGTCTTATTACAGAGGCTTTAAAGTA
CGAAAGGATATTCAAAATATGCACCGGGCTGCCACACTAATTCAGTCATT
CTATCGAATGCACAGGGCCAAAGTTGATTATGAAACAAAGAAAACTGCAA
TTGTGGTTATACAGAATTATTATAGGTTGTATGTTAGAGTAAAAACAGAA
AGAAAAAACTTTTTAGCAGTTCAGAAATCTGTACGAACTATTCAGGCTGC
TTTTAGAGGCATGAAAGTTAGACAAAAATTGAAAAATGTATCAGAGGAAA
AGATGGCAGCCATTGTTAACCAATCTGCACTCTGCTGTTACAGAAGTAAA
ACTCAGTATGAAGCTGTTCAAAGTGAAGGTGTTATGATTCAAGAGTGGTA
TAAAGCTTCTGGCCTTGCTTGTTCACAGGAAGCAGAGTATCATTCTCAAA
GTAGGGCTGCAGTAACAATTCAAAAAGCTTTTTGTAGAATGGTCACAAGA
AAACTGGAAACACAGAAATGTGCTGCCCTACGGATTCAGTTCTTCCTTCA
GATGGCTGTGTATCGGAGAAGATTTGTTCAGCAGAAAAGAGCTGCTATCA
CTTTACAGCATTATTTTAGGACGTGGCAAACCAGAAAACAGTTTTTACTA
TATAGAAAAGCAGCAGTGGTTTTACAAAATCACTACAGAGCATTTCTGTC
TGCAAAACATCAAAGACAAGTCTATTTACAGATCAGAAGCAGTGTTATCA
TTATTCAAGCTAGAAGTAAAGGATTTATACAGAAACGGAAGTTTCAGGAA
ATTAAAAATAGCACCATAAAAATTCAGGCTATGTGGAGGAGATATAGAGC
CAAGAAATATTTATGTAAAGTGAAAGCTGCCTGCAAGATTCAAGCCTGGT
ATAGATGTTGGAGAGCACACAAAGAATATCTAGCTATATTAAAAGCTGTT
AAAATTATTCAAGGTTGCTTCTATACCAAACTAGAGAGAACACGGTTTTT
GAATGTGAGAGCATCAGCAATTATCATTCAGAGAAAATGGAGAGCTATAC
TTCCTGCAAAGATAGCTCATGAACACTTCTTAATGATAAAAAGACATCGA
GCTGCTTGTTTGATCCAAGCACATTATAGAGGATATAAAGGAAGGCAGGT
CTTTCTTCGGCAGAAATCTGCTGCTTTGATCATACAAAAATATATACGAG
CCAGGGAGGCTGGAAAGCATGAAAGGATAAAATATATTGAATTTAAAAAA
TCTACAGTTATCCTACAAGCACTGGTGCGTGGTTGGCTAGTACGAAAAAG
ATTTTTAGAACAGAGAGCCAAAATTCGACTTCTTCACTTCACTGCAGCTG
CATATTATCACCTGAATGCTGTTAGAATTCAAAGAGCCTATAAACTTTAC
CTGGCTGTGAAGAATGCTAACAAGCAGGTTAATTCAGTCATCTGTATTCA
GAGATGGTTTCGAGCAAGATTACAAGAAAAGAGATTTATTCAGAAATATC
ATAGCATCAAAAAGATTGAGCATGAAGGTCAAGAATGTCTGAGCCAGCGA
AATAGGGCTCCATCAGTAATACAGAAAGCAGTGCGCCATTTTCTCCTCCG
TAAAAAGCAGGAAAAATTCACTAGTGGAATCATTAAAATTCAGGCATTAT
GGAGAGGCTATTCTTGGAGGAAGAAAAATGATTCTACAAAAATTAAAGCT
ATACGACTAAGTCTTCAAGTTCTTAATAGGGAGATTCGAGAAGAAAACAA
ACTCTACAAAAGAACTGCACTTGCACTTCATTACCTTTTGACATATAAGC
ACCTTTCTGCCATTCTTGAGGCCTTAAAACACCTAGAGGTAGTTACTAGA
TTGTCTCCACTTTGTTGTGAGAACATGGCCCAGAGTGGAGCAATTTCTAA
AATATTTGTTTTGATCCGAAGTTGTAATCGCAGTATTCCTTGTATGGAAG
TCATCAGATATGCTGTGCAAGTCTTGCTTAATGTATCTAAGTATGAGAAA
ACTACTTCAGCAGTTTATGATGTAGAAAATTGTATAGATATACTATTGGA
GCTTTTGCAGATATACCGAGAAAAGCCTGGTAATAAAGTTGCAGACAAAG
GCGGAAGCATTTTTACAAAAACTTGTTGTTTGTTCGCTATTTTACTGAAG
ACAACAAATAGAGCCTCTGATGTACGAAGTAGGTCCAAAGTTGTTGACCG
TATTTACAGTCTCTACAAACTTACAGCTCATAAACATAAAATGAATACTG
AAAGAATACTTTACAAGCAAAAGAAGAATTCTTCTATAAGCATTCCTTTT
ATCCCAGAAACACCTGTAAGGACCAGAATAGTTTCAAGACTTAAGCCAGA
TTGGGTTTTGAGAAGAGATAACATGGAAGAAATCACAAATCCCCTGCAAG
CTATTCAAATGGTGATGGATACGCTTGGCATTCCTTATTAGTAAATGTAA
ACATTTTCAGTATGTATAGTGTAAAGAAATATTAAAGCCAATCATGAGTA
CGTAAAGTGATTTTTGCTCTCCGTGTACAACTTTTAAAATCTGACTTTGT
TTTAAAAAAACATAAACTGTTCATTACATTCTTCATTTTTATCATTTATA
GTTTTATGCATGTAATAAACTAATATGTCATAAGATGAAAAAAAA
SEQ ID NO: 2 sets out the amino acid sequence
of the human ASPM gene, isoform 1 (NCBI
Reference Sequence: NP_060606.3).
MANRRVGRGCWEVSPTERRPPAGLRGPAEEEASSPPVLSLSHFCRSPFL
CFGDVLLGASRTLSLALDNPNEEVAEVKISHFPAADLGFSVSQRCFVLQP
KEKIVISVNWTPLKEGRVREIMTFLVNDVLKHQAILLGNAEEQKKKKRSL
WDTIKKKKISASTSHNRRVSNIQNVNKTFSVSQKVDRVRSPLQACENLAM
NEGGPPTENNSLILEENKIPISPISPAFNECHGATCLPLSVRRSTTYSSL
HASENRELLNVHSANVSKVSFNEKAVTETSFNSVNVNGQRGENSKLSLTP
NCSSTLNITQSQIHFLSPDSFVNNSHGANNELELVTCLSSDMFMknockd
ownNSQPVHLESTIAHEIYQKILSPDSFIknockdownNYGLNQDLESES
VNPILSPNQFLknockdownNMAYMCTSQQTCKVPLSNENSQVPQSPEDW
RKSEVSPRIPECQGSKSPKAIFEELVEMKSNYYSFIKQNNPKFSAVQDIS
SHSHNKQPKRRPILSATVTKRKATCTRENQTEINKPKAKRCLNSAVGEHE
KVINNQKEKEDFHSYLPHIDPILSKSKSYKNEVTPSSTTASVARKRKSDG
SMEDANVRVAITEHTEVREIKRIHFSPSEPKTSAVKKTKNVTTPISKRIS
NREKLNLKKKTDLSIFRTPISKTNKRTKPIIAVAQSSLTFIKPLKTDIPR
HPMPFAAKNMFYDERWKEKQEQGFTWWLNFILTPDDFTVKINISEVNAAT
LLLGIENQHKISVPRAPTKEEMSLRAYTARCRLNRLRRAACRLFTSEKMV
KAIKKLEIEIEARRLIVRknockdownRHLWknockdownVGERQKVLNW
LLSYNPLWLRIGLETTYGELISLEDNSDVTGLAMFILNRLLWNPDIAAEY
RHPTVPHLYRDGHEEALSKFTLKKLLLLVCFLDYAKISRLIDHDPCLFCk
nockdownAEFKASKEILLAFSRDFLSGEGDLSRHLGLLGLPVNHVQTPF
DEFDFAVTNLAVDLQCGVRLVRTMELLTQNWDLSKKLRIPAISRLQKMHN
VDIVLQVLKSRGIELSDEHGNTILSknockdownIVDRHREKTLRLLWKI
AFAFQVDISLNLDQLKEEIAFLKHTKSIKKTISLLSCHSDDLINKKKGKR
DSGSFEQYSENIKLLMDWVNAVCAFYNKKVENFTVSFSDGRVLCYLIHHY
HPCYVPFDAICQRTTQTVECTQTGSVVLNSSSESDDSSLDMSLKAFDHEN
TSELYKELLENEKKNFHLVRSAVRDLGGIPAMINHSDMSNTIPDEKVVIT
YLSFLCARLLDLRKEIRAARLIQTTWRKYKLKTDLKRHQEREKAARIIQL
AVINFLAKQRLRKRVNAALVIQKYWRRVLAQRKLLMLKKEKLEKVQNKAA
SLIQGYWRRYSTRQRFLKLKYYSIILQSRIRMIIAVTSYKRYLWATVTIQ
RHWRAYLRRKQDQQRYEMLKSSTLIIQSMFRKWKQRKMQSQVKATVILQR
AFREWHLRKQAKEENSAIIIQSWYRMHKELRKYIYIRSCVVIIQKRFRCF
QAQKLYKRRKESILTIQKYYKAYLKGKIERTNYLQKRAAAIQLQAAFRRL
KAHNLCRQIRAACVIQSYWRMRQDRVRFLNLKKTIIKFQAHVRKHQQRQK
YKKMKKAAVIIQTHFRAYIFAMKVLASYQKTRSAVIVLQSAYRGMQARKM
YIHILTSVIKIQSYYRAYVSKKEFLSLKNATIKLQSTVKMKQTRKQYLHL
RAAALFIQQCYRSKKIAAQKREEYMQMRESCIKLQAFVRGYLVRKQMRLQ
RKAVISLQSYFRMRKARQYYLKMYKAIVIQNYYHAYKAQVNQRKNFLQVK
KAATCLQAAYRGYKVRQLIKQQSIAALKIQSAFRGYNKRVKYQSVLQSII
KIQRWYRAYKTLHDTRTHFLKTKAAVISLQSAYRGWKVRKQIRREHQAAL
KIQSAFRMAKAQKQFRLEKTAALVIQQNFRAWTAGRKQCMEYIELRHAVL
VLQSMWKGKTLRRQLQRQHKCAIIIQSYYRMHVQQKKWKIMKKAALLIQK
YYRAYSIGREQNHLYLKTKAAVVTLQSAYRGMKVRKRIknockdownCNK
AAVTIQSKYRAYKTKKKYATYRASAHIIQRWYRGIKITNHQHKEYLNLKK
TAIKIQSVYRGIRVRRHIQHMHRAATFIKAMFKMHQSRISYHTMRKAAIV
IQVRCRAYYQGKMQREKYLTILKAVKVLQASFRGVRVRRTLRKMQTAATL
IQSNYRRYRQQTYFNKLKKITKTVQQRYWAMKERNIQFQRYNKLRHSVIY
IQAIFRGKKARRHLKMMHIAATLIQRRFRTLMMRRRFLSLKKTAILIQRK
YRAHLCTKHHLQFLQVQNAVIKIQSSYRRWMIRKRMREMHRAATFIQSTF
RMHRLHMRYQALKQASVVIQQQYQANRAAKLQRQHYLRQRHSAVILQAAF
RGMKTRRHLKSMHSSATLIQSRFRSLLVRRRFISLKKATIFVQRKYRATI
CAKHKLYQFLHLRKAAITIQSSYRRLMVKKKLQEMQRAAVLIQATFRMYR
TYITFQTWKHASILIQQHYRTYRAAKLQRENYIRQWHSAVVIQAAYKGMK
ARQLLREKHKASIVIQSTYRMYRQYCFYQKLQWATKIIQEKYRANKKKQK
VFQHNELKKETCVQAGFQDMNIKKQIQEQHQAAIIIQKHCKAFKIRKHYL
HLRATVVSIQRRYRKLTAVRTQAVICIQSYYRGFKVRknockdownIQNM
HRAATLIQSFYRMHRAKVDYETKKTAIVVIQNYYRLYVRVKTERKNFLAV
QKSVRTIQAAFRGMKVRQKLKNVSEEKMAAIVNQSALCCYRSKTQYEAVQ
SEGVMIQEWYKASGLACSQEAEYHSQSRAAVTIQKAFCRMVTRKLETQKC
AALRIQFFLQMAVYRRRFVQQKRAAITLQHYFRTWQTRKQFLLYRKAAVV
LQNHYRAFLSAKHQRQVYLQIRSSVIIIQARSKGFIQKRKFQEIKNSTIK
IQAMWRRYRAKKYLCKVKAACKIQAWYRCWRAHKEYLAILKAVKIIQGCF
YTKLERTRFLNVRASAIIIQRKWRAILPAKIAHEHFLMIKRHRAACLIQA
HYRGYKGRQVFLRQKSAALIIQKYIRAREAGKHERIKYIEFKKSTVILQA
LVRGWLVRKRFLEQRAKIRLLHFTAAAYYHLNAVRIQRAYKLYLAVKNAN
KQVNSVICIQRWFRARLQEKRFIQKYHSIKKIEHEGQECLSQRNRAASVI
QKAVRHFLLRKKQEKFTSGIIKIQALWRGYSWRKKNDCTKIKAIRLSLQV
VNREIREENKLYKRTALALHYLLTYKHLSAILEALKHLEVVTRLSPLCCE
NMAQSGAISKIFVLIRSCNRSIPCMEVIRYAVQVLLNVSKYEKTTSAVYD
VENCIDILLELLQIYREKPGNKVADKGGSIFTKTCCLLAILLKTTNRASD
VRSRSKVVDRIYSLYKLTAHKHKMNTERILYKQKKNSSISIPFIPETPVR
TRIVSRLKPDWVLRRDNMEEITNPLQAIQMVMDTLGIPY
SEQ ID NO: 3 sets out the nucleotide sequence
of the mRNA encoded by exon 18 of human ASPM
gene, which corresponds to nucleotide 4323 to
nucleotide 9077 of the ASPM transcript variant 1
(NCBI Reference Sequence: NM_018136.4).
GGATATTGGAGAAGATATTCCACTAGACAAAGATTTCTGAAATTGAAATA
TTATTCAATCATCCTGCAATCTAGGATAAGAATGATAATTGCTGTTACAT
CTTATAAACGATATCTTTGGGCTACAGTTACAATTCAGAGGCATTGGCGT
GCTTATTTAAGAAGAAAACAAGATCAACAAAGATATGAAATGCTAAAATC
ATCAACTCTTATAATCCAATCTATGTTCAGAAAATGGAAGCAACGTAAAA
TGCAATCACAAGTAAAAGCTACAGTAATATTGCAAAGAGCTTTTAGAGAA
TCGCATTTAAGAAAACAAGCTAAAGAAGAAAATTCTGCTATTATCATACA
ATCATGGTATAGAATGCATAAAGAATTACGGAAATATATTTATATTAGAT
CTTGTGTTGTTATCATTCAGAAAAGATTTCGGTGCTTTCAAGCCCAAAAG
TTATATAAAAGAAGAAAAGAGTCCATACTAACCATCCAGAAGTACTACAA
AGCATATCTGAAAGGAAAGATTGAGCGCACCAACTATTTGCAGAAACGAG
CTGCAGCCATTCAATTACAAGCTGCTTTTAGGAGACTGAAAGCTCATAAT
TTATGTAGACAAATTAGAGCTGCTTGTGTTATTCAGTCATACTGGAGAAT
GAGACAAGACAGAGTTCGATTTTTAAACCTTAAGAAGACTATTATCAAAT
TTCAGGCACATGTAAGAAAACATCAACAACGACAGAAATATAAGAAGATG
AAGAAAGCAGCTGTTATAATTCAGACTCATTTCCGAGCTTATATTTTTGC
CATGAAAGTTCTAGCATCTTACCAGAAAACACGCTCTGCTGTCATTGTGC
TGCAGTCTGCATATAGAGGGATGCAAGCCAGGAAAATGTATATTCACATC
CTCACATCTGTTATAAAGATTCAATCATATTATCGTGCTTATGTTTCTAA
AAAGGAATTTTTGAGCCTAAAAAATGCTACAATAAAATTGCAGTCAACTC
TTAAGATGAAACAAACACGTAAACAATATTTGCATTTAAGAGCAGCTGCA
CTATTTATCCAGCAATGTTACCGTTCCAAAAAAATAGCTGCACAAAAGAG
AGAAGAGTATATGCAGATCCGGGAATCTTGTATCAAACTGCAAGCATTTG
TTAGAGGATACCTTGTCCGAAAGCAGATGAGGTTACAAAGAAAAGCTGTT
ATTTCACTACAGTCTTATTTCAGAATGAGAAAGGCTCGGCAGTATTATCT
GAAAATGTATAAAGCAATTATTGTCATTCAGAATTACTATCATGCATACA
AAGCACAGGTCAATCAGAGGAAGAACTTCTTGCAAGTCAAAAAAGCAGCT
ACTTGCTTGCAAGCAGCTTACAGAGGTTATAAAGTACGCCAGCTAATCAA
ACAACAATCTATAGCTGCTCTTAAAATTCAGTCTGCTTTTAGAGGCTATA
ATAAAAGGGTAAAATATCAATCTGTGCTTCAATCTATAATAAAGATTCAG
AGATGGTACAGGGCGTACAAGACTCTTCATGATACAAGAACACATTTTTT
GAAGACAAAGGCAGCTGTGATTTCCCTCCAGTCTGCTTATCGTGGCTGGA
AGGTTCGGAAACAGATTAGAAGGGAACATCAAGCTGCCTTGAAGATTCAG
TCTGCTTTTAGAATGGCCAAGGCCCAGAAACAGTTTAGATTGTTTAAAAC
AGCAGCATTAGTCATCCAGCAAAATTTCAGAGCATGGACTGCAGGAAGGA
AGCAATGTATGGAGTATATTGAACTCCGTCATGCGGTACTGGTGCTTCAA
TCTATGTGGAAGGGAAAAACACTGAGAAGACAGCTTCAAAGGCAACATAA
ATGTGCTATCATCATACAGTCATACTATAGAATGCATGTGCAACAAAAGA
AGTGGAAAATCATGAAAAAAGCTGCTCTTCTGATTCAAAAGTATTATAGG
GCTTACAGTATTGGAAGAGAACAGAATCATTTATATTTGAAAACAAAAGC
AGCTGTAGTAACTTTACAGTCAGCTTATCGTGGTATGAAAGTGAGAAAAA
GAATAAAGGATTGCAACAAAGCAGCAGTCACTATACAGTCTAAATACAGA
GCTTACAAAACCAAAAAGAAATATCCAACCTATAGAGCTTCAGCTATTAT
AATTCAGAGATCGTATCGAGGTATTAAAATTACAAACCATCAGCATAAGG
AGTATCTTAATTTGAAGAAGACAGCAATTAAAATCCAATCTGTTTATAGA
GGTATTAGAGTTAGAAGACATATTCAACACATGCACAGGGCAGCCACTTT
TATTAAAGCCATGTTTAAAATGCATCAGTCAAGAATAAGTTACCATACAA
TGAGAAAAGCAGCTATTGTTATTCAAGTAAGATGTAGAGCATATTATCAA
GGTAAAATGCAGCGTGAAAAGTACCTGACAATTTTGAAAGCTGTTAAAGT
CCTTCAGGCAAGTTTTAGAGGAGTAAGAGTTAGACGGACTCTTAGAAAGA
TGCAGACTGCAGCAACACTCATTCAGTCAAACTACAGAAGATACAGACAG
CAAACATACTTTAATAAGTTAAAGAAAATAACAAAAACAGTACAGCAAAG
ATACTGGGCAATGAAAGAAAGAAACATACAATTTCAAAGGTATAACAAAC
TGAGGCATTCTGTAATATACATTCAGGCTATTTTTAGGGGAAAGAAAGCT
AGAAGACATTTAAAAATGATGCATATAGCCGCAACTCTCATTCAGAGGAG
ATTTAGAACTCTAATGATGAGAAGAAGATTCCTCTCTCTCAAGAAAACTG
CTATTTTGATTCAGAGAAAATATCGGGCACATCTTTGTACAAAGCATCAC
TTACAGTTCCTTCAGGTACAAAATGCAGTTATTAAAATCCAGTCATCATA
CAGAAGATGGATGATAAGGAAAAGGATGCGAGAGATGCACAGGGCTGCTA
CTTTCATCCAGTCTACTTTCAGAATGCACAGATTACATATGAGATATCAG
GCTTTGAAACAGGCCTCCGTTGTGATCCAACAGCAATACCAAGCAAATAG
AGCTGCAAAACTGCAGAGGCAGCATTATCTCAGACAAAGACACTCTGCTG
TGATCCTTCAGGCTGCATTCAGGGGTATGAAAACTAGAAGACATTTGAAG
AGTATGCATTCCTCTGCAACCCTTATTCAGAGTAGGTTTAGATCATTACT
GGTGAGGAGAAGATTCATTTCCCTCAAAAAAGCTACTATTTTTGTTCAGA
GGAAATATCGAGCCACCATTTGTGCCAAACATAAATTGTACCAATTCTTG
CACTTAAGAAAGGCAGCCATTACAATACAGTCATCTTACAGAAGACTGAT
GGTAAAGAAGAAGTTACAAGAAATGCAAAGGGCTGCAGTTCTCATTCAGG
CTACTTTCAGGATGTACAGAACATATATTACATTTCAGACTTGGAAACAT
GCTTCAATTCTAATTCAGCAACATTATCGAACATATAGAGCTGCAAAATT
ACAAAGAGAAAATTATATCAGACAATGGCATTCTCCTGTGGTTATTCAGG
CTGCATATAAAGGAATGAAAGCAAGACAACTTTTAAGGGAAAAACACAAA
GCTTCTATCGTAATACAAAGCACCTACAGAATGTATAGGCAGTATTGTTT
CTACCAAAAGCTTCAGTGGGCTACAAAAATCATACAAGAAAAATATAGAG
CAAATAAAAAGAAACAGAAAGTATTTCAACACAATGAACTTAAGAAAGAG
ACTTGTGTTCAGGCAGGTTTTCAGGACATGAACATAAAAAAACAGATTCA
GGAACAGCACCAGGCTGCCATTATTATTCAGAAGCATTGTAAAGCCTTTA
AAATAAGGAAGCATTATCTCCACCTTAGAGCAACAGTAGTTTCTATTCAA
AGAAGATACAGAAAACTAACTGCAGTGCGTACCCAAGCAGTTATTTGTAT
ACAGTCTTATTACAGAGGCTTTAAAGTACGAAAGGATATTCAAAATATGC
ACCGGGCTGCCACACTAATTCAGTCATTCTATCGAATGCACAGGGCCAAA
GTTGATTATGAAACAAAGAAAACTGCAATTGTGGTTATACAGAATTATTA
TAGGTTGTATGTTAGAGTAAAAACAGAAAGAAAAAACTTTTTAGCAGTTC
AGAAATCTGTACGAACTATTCAGGCTGCTTTTAGAGGCATGAAAGTTAGA
CAAAAATTGAAAAATGTATCAGAGGAAAAGATGGCAGCCATTGTTAACCA
ATCTGCACTCTGCTGTTACAGAAGTAAAACTCAGTATGAAGCTGTTCAAA
GTGAAGGTGTTATGATTCAAGAGTGGTATAAAGCTTCTGGCCTTGCTTGT
TCACAGGAAGCAGAGTATCATTCTCAAAGTAGGGCTGCAGTAACAATTCA
AAAAGCTTTTTGTAGAATGGTCACAAGAAAACTGGAAACACAGAAATGTG
CTGCCCTACGGATTCAGTTCTTCCTTCAGATGGCTGTGTATCGGAGAAGA
TTTGTTCAGCAGAAAAGAGCTGCTATCACTTTACAGCATTATTTTAGGAC
GTGGCAAACCAGAAAACAGTTTTTACTATATAGAAAAGCAGCAGTGGTTT
TACAAAATCACTACAGAGCATTTCTGTCTGCAAAACATCAAAGACAAGTC
TATTTACAGATCAGAAGCAGTGTTATCATTATTCAAGCTAGAAGTAAAGG
ATTTATACAGAAACGGAAGTTTCAGGAAATTAAAAATAGCACCATAAAAA
TTCAG
SEQ ID NO: 4 sets out the nucleotide sequence
of the sense strand of the ASPM-vl-targeted
siASPM-v1.4822, which corresponds to nucleotide
4822 to nucleotide 4840 of the ASPM
transcript variant 1.
GCGCACCAACUAUUUGCAG
SEQ ID NO: 5 sets out the nucleotide sequence
of the antisense strand of the ASPM-v1-targeted
siASPM-v1.4822, which is complimentary to the
sense strand shown in SEQ ID NO: 4.
CUGCAAAUAGUUGGUGCGC
SEQ ID NO; 6 sets out the nucleotide sequence
of the sense strand of the ASPM-vl-targeted
siASPM-v1.7636, which corresponds to nucleotide
7636 to nucleotide 7654 of the ASPM
transcript variant 1.
CAGAAGACUGAUGGUAAAG
SEQ ID NO: 7 sets out the nucleotide sequence
of the antisense strand of the ASPM-vl-targeted
siASPM-v1.7636, which is complimentary to the
sense strand shown in SEQ ID NO; 6,
CUUUACCAUCAGUCUUCUG
SEQ ID NO: 8 sets out the nucleotide sequence
of the sense strand of the ASPM-v1-targeted
siASPM-v1.4360, which corresponds to nucleotide
4360 to nucleotide 4378 of the ASPM
transcript variant 1.
CCUGCAAUCUAGGAUAAGA
SEQ ID NO: 9 sets out the nucleotide sequence
of the antisense strand of the ASPM-v1-targeted
siASPM-v1.4360, which is complimentary to the
sense strand shown in SEQ ID NO: 8.
GGACGUUAGAUCCUAUUCU

### EXAMPLE

The following examples are given for illustrative purposes only and are not intended to be limiting unless otherwise specified. It should be appreciated by those of skill in the art that the techniques disclosed in the examples which follow represent techniques discovered by the inventors to function well in the practice of invention, and thus can be considered to constitute preferred modes for its practice. Those of skill in the art should appreciate that many changes can be made in the specific embodiments which are disclosed and still obtain a like or similar result without departing from the spirit and scope of the invention.

### Example 1 Identification of ASPM as the Top-Ranked Wat-Related Gene that is Associated with Cancer Metastasis

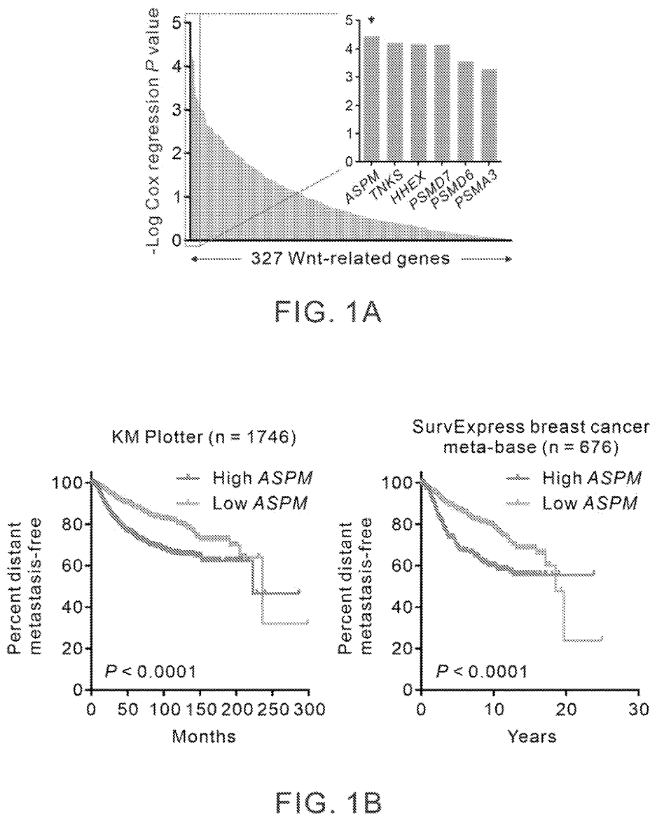

We focus on a list of 327 Wnt-related genes according to Gene Ontology terms. When ranked descendingly according to their prognostic significance (Cox regression P-value), the gene whose transcript levels correlate the best with shorter relapse-free survival (i.e., the highest probability of relapse and/or metastasis) is ASPM (P<0.0001; FIG. 1A). Consistently, we found that the ASPM expression inversely correlates with distant-metastasis-free survival in large meta-analyses of patient cohorts (total n=2422; P<0.0001; FIG. 1B).

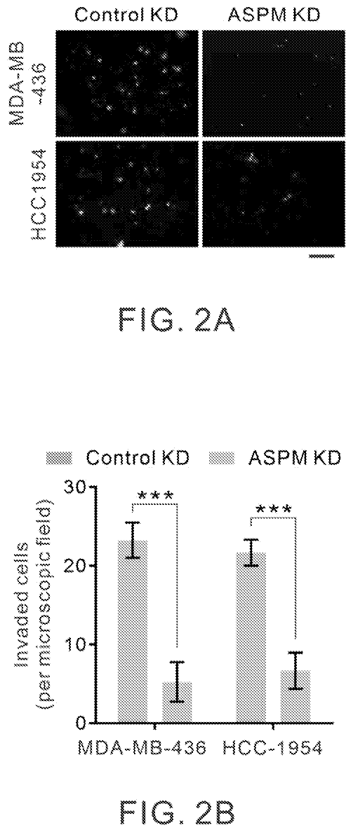

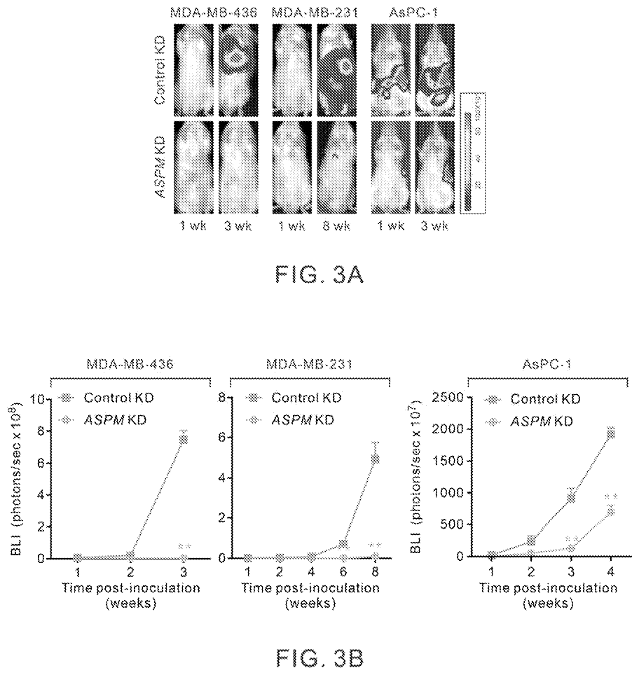

The preceding clinical correlative analysis raised the possibility that ASPM may play a functional role in breast cancer invasiveness and metastasis. Indeed, small hairpin RNA (shRNA)-mediated knockdown (knockdown) of ASPM expression in MDA-MB-436 and HCC-1954 breast cancer cells completely abrogated their ability to invade through the recombinant basement membrane (FIG. 2). To verify the functional importance of ASPM expression in vivo, human breast cancer MDA-MB-436 cells and MDA-MB-231 cells and pancreatic cancer AsPC-1 cells were lentivirally transduced a GFP and firefly luciferase (FF-Luc) fusion vector (UBC-EGFP-T2A-Luc; System Biosciences) and GFP-positive cells were enriched by FACS. MDA-MB-436-FF-Luc or MDA-MB-231-FF-Lue cells (10″ cells) that were lentivirally infected with control- or ASPM-specific shRNA (control knockdown or ASPM knockdown) were injected through tail veins into immunodeficient NOD/SCID mice through a 27-gauge needle. The resultant pulmonary metastatic tumors were visualized by bioluminescence imaging (BLI) weekly, starting from 3 weeks following cell inoculation, according to the manufacturer's recommendations (IVIS Imaging System. Caliper Life Sciences). As shown in FIGS. 3, the control knockdown breast cancer cells led to the development of metastatic pulmonary tumors within 3 weeks or 8 weeks following cell injection. By contrast, knockdown of ASPM expression almost completely abrogated the distant metastases. We further verified the findings in a distant metastasis model of pancreatic cancer. Briefly, AsPC-1-FF-Luc cells were injected into the splenic pulp of NOD/SCID mice over 1 minute, followed by splenectomy and splenic vein ligation. The development of hepatic and/or metastatic peritoneal tumors was monitored by BLI. In accordance with the preceding findings in breast cancer, control knockdown pancreatic cancer AsPC-1 cells rapidly disseminated into the liver and the peritoneum 3 weeks following cell inoculation, whereas knockdown of ASPM expression substantially nullified the pro-metastatic ability of the cancer cells.

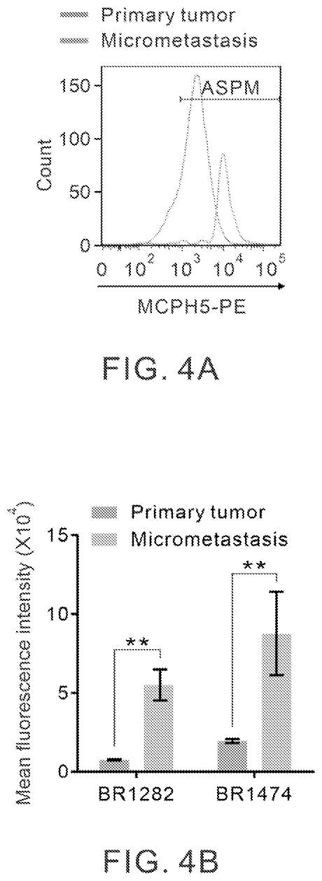

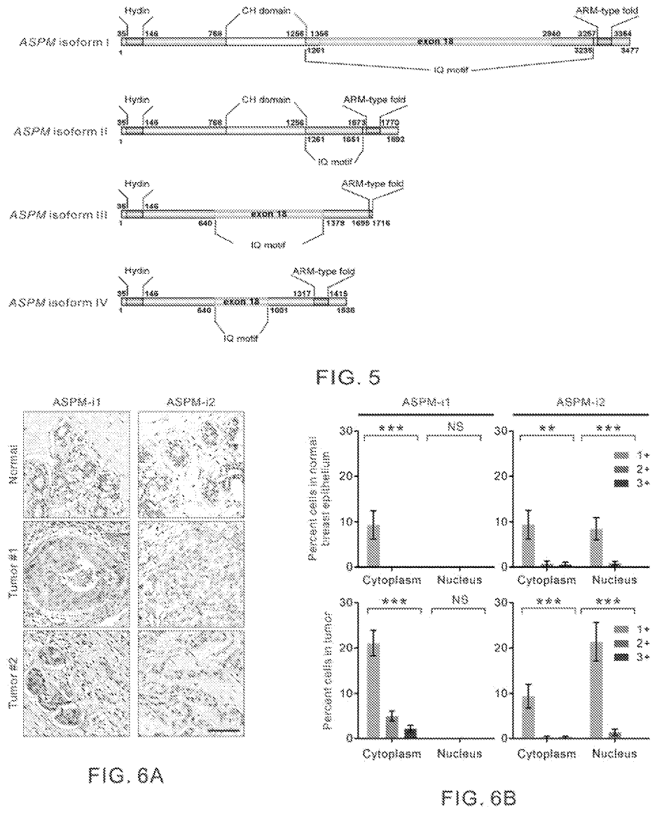

Compatible with the prevailing theory proposing that metastatic colonization is seeded by rare tumor cells with unique properties (Lawson, D. A., Bhakta, N. R., Kessenbrock, K., Prummel, K. D., Yu, Y., Takai, K., Zhou, A., Eyoh, H., Balakrishnan, S., Wang, C. Y., et al. (2015). Single-cell analysis reveals a stem-cell program in human metastatic breast cancer cells. Nature 526, 131-135), we sought to substantiate this finding by establishing patient-derived xenograft (PDX) models of breast cancer and analyzing the ASPM expression in micro-metastatic lesions. Briefly, PDX breast tumors derived from two human TNBC tumors were implanted into the mammary fat pads of NOD/Shi-scid/IL2Rγ^{null} (NOG) mice (BR 1282 and BR1474, CrownBio), and the tumors were calipered weekly to monitor growth kinetics. The lung tissues were harvested from the tumor-bearing mice when the tumor reached 2 mm³, at which time the development of pulmonary micro-metastases was confirmed by parallel experiments. The resected tissues were then digested and the cells were labeled with APC-anti-CD298, which is a specific surface marker of human breast cancer cells (Lawson et al., 2015) (BioLegend 341706), then fixed for the subsequent labeling with PE-anti-ASPM (Santa Cruz, se-48883), and analyzed by fluorescence-activated cell sorting (FACS). Indeed, cancer cells isolated from the micro-metastatic niches in the lungs of mice bearing PDX expressed high levels of ASPM compared with those in the parental tumors (FIG. 4). Taken together, these in vitro and in vivo findings support ASPM as an important and indispensable factor of cancer invasiveness and metastasis.

### Example 2 Specific Upregulated Expression of ASPM Protein Isoform 1 in Malignant Tumors

Several splicing variants of the ASPM transcripts exist in normal and malignant human tissues, which encode the protein isoforms consisting of 3477 (isoform 1; ASPM-i1), 1892 (isoform 2; ASPM-i2), 1389 (isoform 21), and 1062 amino acid residues (isoform IV), respectively (Kouprina, N., Pavlicek, A., Collins, N. K., Nakano, M., Noskov, V. N., Ohzeki, J., Mochida, G. H., Risinger, J. I., Goldsmith, P., Gunsior, M., et al. (2005). The microcephaly ASPM gene is expressed in proliferating tissues and encodes for a mitotic spindle protein. Hum Mol Genet 14, 2155-2165). Importantly, the three shorter ASPM isoforms (isoforms 2-4) lack multiple functional domains of the full-length isoform 1 protein, such as the IQ (isoleucine and glutamine) motifs and the calponin-homology (CH) domain, raising the possibility that these ASPM isoforms may play different roles in normal and malignant cells. Notably, the transcript variant 1 (ASPM-v1; NCBI RefSeq: NM_018136.4), which encodes the full-length ASPM protein (ASPM isoform 1 or ASPM-i1; NCBI RefSeq: NP_060606.3), and variant 2 (NCBI RefSeq: NM_001206846), which encodes a truncated protein lacking the 67 IQ domains carried by exon 18 (ASPM-i1; NCBI RefSeq: NP_001193775.1), are the two major transcripts detected in pancreatic cancer cells (Hsu et al., 2019a). In several types of cancer, ASPM augments canonical Wint signaling by positively regulating critical upstream Wnt mediators, including dishevelled (DVL) proteins and β-catenin (Wang, W. Y., Hsu, C. C., Wang, T. Y., Li, C. R., Hou, Y. C., Chu, J. M., Lee, C. T., Lìu, M. S., Su, J. J., Jian, K. Y., et al. (2013). A gene expression signature of epithelial tubulogenesis and a role for ASPM in pancreatic tumor progression. Gastroenterology 145, 1110-1120). Interestingly, in pancreatic cancer, using a rabbit polyclonal antibody raised using a peptide epitope located within the fragment encoded by the exon 18 of the human ASPM gene (unique to ASPM-v1 and ASPM-i1), it was demonstrated that only ASPM-i1 associated with DVL.2 and regulates its protein stability in cancer cells. By contrast, the protein isoform 2 of ASPM interacts with cyclin E and thereby regulates cell cycle progression in cancer cells. As such, ASPM-i1 but not ASPM-isoform 2 contributes to the Wat activity and the tumorigenicity of cancer cells, such as pancreatic cancer (Hsu, C. C., Liao, W. Y., Chan, T. S., Chen, W. Y., Lee, C. T., Shan, Y. S., Huang, P. J., Hou, Y. C., Li. C. R., and Tsai, K. K. (2019a). The differential distributions of ASPM isoforms and their roles in Wnt signaling, cell cycle progression, and pancreatic cancer prognosis. J Pathol 249, 498-508). These results suggest that different ASPM isoforms have different functions and pathogenetic roles in cancer cells.

Previously, ASPM-i1 has been shown to be specifically expressed in the cytoplasm of pancreatic cancer cells, whereas ASPM-i2 is mainly expressed in cell nuclei (Hsu et al., 2019a). Importantly, immunohistochemical (IHC) analysis on tissue microarrays (TMA) of human pancreatic or gastric cancer tissues revealed that the expression of ASPM-i1 is upregulated in tumor cells, whereas its staining is rarely positive in the normal pancreatic ductal epithelium (data not shown). To extend these observations to non-gastroenterological (GI) tract malignant tumors, TMA analysis was conducted on human breast cancer tissues and the matched adjacent normal tissues (BR084b, US Biomax, Inc., Rockville, MD. USA). Tissue sections were deparaffinized, hydrated, and immersed in citrate buffer at pH 6.0 for epitope retrieval in a microwave. Endogenous peroxidase activity was quenched in 3% hydrogen peroxidase for 15 minutes, and slides were then incubated with 10% normal horse serum to block nonspecific immunoreactivity. We raised rabbit polyclonal antibodies (1:1600) against immunogens specific for ASPM-i1 or ASPM-i2. The staining intensities were quantified at the single-cell level, with at least 300 tumor cells counted per tumor (3 tissue sections per tumor; at least 100 tumor cells counted per section).

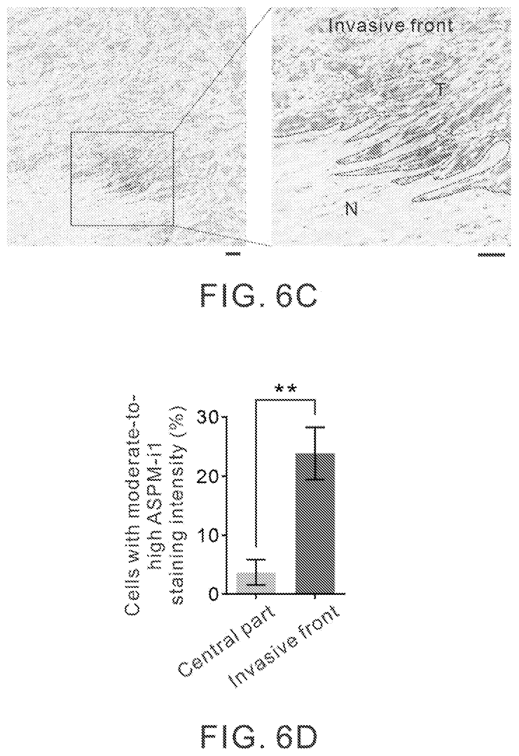

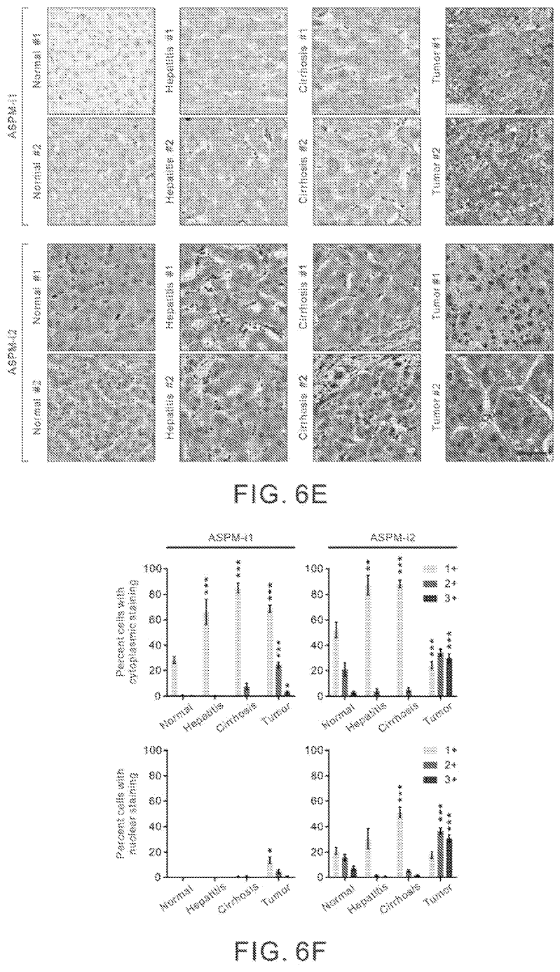

As shown in FIGS. 6A, a small subset (approximately 9.3%) of the epithelial cells in normal human breast tissues display a weak (1+) staining intensity of ASPM-i1 in the cytoplasm, whereas ASPM-i2 is expressed (≥1+) in either the cytoplasm (10.5%) or the nuclei (9.3%) of a small proportion of mammary epithelial cells. In accordance with the preceding findings in pancreatic cancers, the expression of ASPM-i1 is markedly upregulated in the cytoplasm of the malignant cells in breast cancer tissues. By contrast, the expression of ASPM-i2 remains predominantly in the cell nuclei of breast cancer cells. Specifically, an average of approximately 21.1% of the tumor cells exhibit a weak (1+) staining of ASPM-i1, with approximately 5.0% of them exhibiting moderate (2+) staining and approximately 2.2% of them exhibiting strong (3+) staining within the same tumor. By contrast, there are an average of 9.4%, 0.3%, and 0.3%, respectively, of the tumor cells, exhibiting weak, moderate, and strong staining of ASPM-i2, respectively, in the cytoplasm (FIG. 6B). To further examine the expression pattern of ASPM-i1 in breast cancer, we repeated its IHC staining in the whole-tumor tissue sections of 3 patients with breast cancer. Intriguingly and importantly, we noted a significant regional heterogeneity in the expression level of ASPM-i1 in breast cancer tissues, which is higher in the cancer invasive front than in other regions (FIG. 6C and FIG. 6D). This observation raised the possibility that ASPM-i1 may be linked to breast cancer invasiveness and/or metastasis.

Having demonstrated the expressional heterogeneity of ASPM isoforms, we next sought to determine if the expression pattern of ASPM, especially the specific upregulation of ASPM-i1 in the cytoplasm of cancer cells, can be generalized to other types of human cancers. Previously, the transcript level of ASPM has been shown to increase in 66% of human HCC tissues and correlates with short S-year survival and early tumor recurrence in HCC patients (Lin, S. Y., Pan, H. W., Liu, S. H., Jeng, Y. M., Hu, F. C., Peng, S. Y., Lai, P. I., and Hsx, H. C. (2008). ASPM is a novel marker for vascular invasion, early recurrence, and poor prognosis of hepatocellular carcinoma. Clin Cancer Res 14, 4814-4820). To investigate the expression pattern of ASPM-i1 in human normal and malignant liver tissues at the protein level, we carried out IHC staining of ASPM-i1 on two tissue microarrays consisting of 24 normal liver tissues, 10 hepatitis liver tissues, 50 cirrhotic liver tissues, and 111 HCC tissues (LVN241a and LV805b, US Biomax, Inc., Rockville, MD, USA). Like its expression pattern in breast cancer tissues, ASPM-i1 is mainly expressed (≥1+) in the cytoplasm of a subset (averaged 28.3%) of normal hepatocytes in normal liver tissues (FIG. 6E), and the percentage of epithelial cells with weak (1+) ASPM-i1 staining significantly increased in hepatitis, cirrhotic, and HCC tissues. Importantly, a significant population of epithelial cells express a moderate (2+; averaged 24.3%) or a strong (3+; averaged 3.43%) staining intensity of ASPM-i1 in HCC tissues. By contrast, the expression of ASPM-i1 in cell nuclei in normal liver, hepatitis, and cirrhotic liver tissues was neglectable. In HCC tissues, only a small percentage of tumor cells express weak (1+; averaged 13.5%) or moderate (2+; averaged 4.7%) staining of ASPM-i1 in their nuclei.

In contrast to the staining pattern of ASPM-i1 in liver tissues, the majority of hepatocytes in the normal liver express ASPM-i2 (≥1+) in the cytoplasm (76.0%), and a large proportion of hepatocytes also express ASPM-i2 in cell nuclei (44.1%) (FIG. 6E). Interestingly, the expression of ASPM-i2 is moderately upregulated in HCC tissues, with approximately two-thirds of tumor cells displaying a moderate-to-strong (≥2+) staining intensity of ASPM-i2 in the cytoplasm (64.0%) or cell nuclei (67.5%) (FIG. 6F).

Collectively, the very low frequency of expression of ASPM-i1 in normal epithelial tissues, together with its specific upregulation in cancer cells, support it as a cancer-cell specific target that has a favorable safety profile in the treatment of cancers.

### Example 3 ASPM Isoform 1 (ASPM-i1) Specifically Promotes the Assembly of the PAR-Planar Cell Polarity Complex and Invadopodia Formation in Invasive Cancer Cells

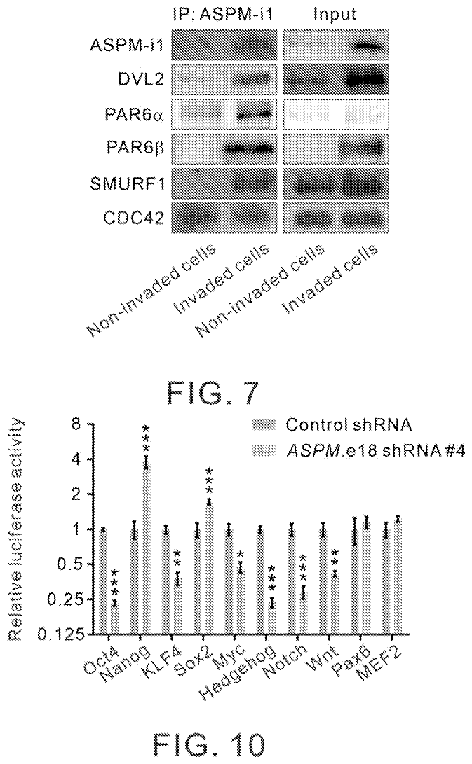

Our preceding findings of the important roles of ASPM in the invasiveness of cancer cells and cancer metastasis, together with the specific role of ASPM-i1 in Wnt activity and tumorigenesis, incited us to gain mechanistic insights into how it regulates Wnt-associated invasiveness in cancer cells. To this end, we isolated invasive breast cancer MDA-MB-436 cells using a modified Boyden chamber invasion assay (Transwell inserts; BD Biosciences). The Transwell inserts were coated with a thin layer of growth factor-reduced reconstituted basement membrane (rBM; BD Biosciences), and the cells were allowed to invade across the rBM for 12 hours. The cells that invaded through the insert membrane and those that stayed above the membrane were collected, respectively. We then screened a list of 107 annotated human Wat proteins (http://web.stanford. edu/group/nasselab/cgi-bin/wnt/) using a high-throughput Micro Western Array analysis (Ciaccio, M. F., Wagner, J. P., Chou, C. P., Lauffenburger, D. A., and Jones, R. B. (2010). Systems analysis of EGF receptor signaling dynamics with microwestern arrays. Nat Methods 7, 148-155) for interaction with ASPM-i1 in invasive breast cancer. Several members in the PAR-planar cell polarity (PCP) protein complex, including Par-6 family cell polarity regulator alpha (PAR6α), PAR6β, disheveled 2 (DVL2), CDC42, and SMURF1, strongly interact with ASPM-i1 (FIG. 7).

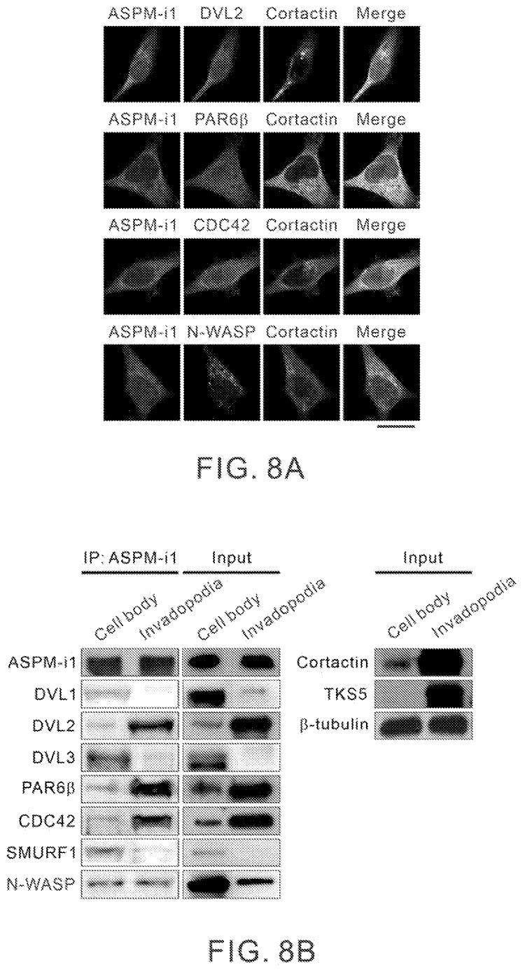

Since the noncanonical Wnt-PCP signaling translates tissue patterning information to individual cells where it controls cell morphogenetic behaviors as well the invasive behaviors of malignant cells through regulating cell polarity and invadopodia (Luga, V., Zhang, L., Viloria-Petit, A. M., Ogunjimi, A. A., Inanlou, M. R., Chiu, E., Buchanan, M., Hosein, A. N., Basik, M., and Wrana, J. L. (2012). Exosomes mediate stromal mobilization of autocrine Wni-PCP signaling in breast cancer cell migration. Cell 151, 1542-1556), we considered the possibility that ASPM-i1 may coordinate the PAR-PCP proteins to regulate invadopodia dynamics and thus cancer cell invasiveness. We thus induced invadopodia formation of breast cancer MDA-MB-436 cells by plating them onto the gelatin matrix (Sigma-Aldrich) as described previously (Eckert, M. A., Lwin, T. M., Chang, A. T., Kim, J., Dams, E., Ohno-Machado, L., and Yang, J. (2011). Twist1-induced unvadopodia formation promotes tumor metastasis. Cancer Cell 19, 372-386). The cells were seeded on gelatin for 3 hours or longer and then immunostained with anti-cortactin and Alexa Fluor 647 phalloidin (staining for F-actin) and evaluated the staining patterns using confocal imaging analysis. The cortactin “F-actin” structures protruding downwardly from the cells are considered invadopodia. As shown in FIGS. 8A, confocal imaging analyses revealed that ASPM-i1 colocalizes with DVL2, PAR6β, CDC42, and cortactin in the invadopodia of invasive cancer cells. To confirm that ASPM-i1 interacts with the PAR/PCP proteins, specifically in invadopodia, we isolated invadopodia proteins from cells plated on the gelatin matrix for 3 hours to induce the formation of invadopodia. Cell bodies were sheared from the surface 2.5 of the plates to leave the invadopodia embedded in the gelatin. The invadopodia protein and the cell body protein fractions were then solubilized in IP buffer. For co-IP, cells were lysed by non-denaturing lysis buffer (1 mM PMSF, 1 mM Na3VO4, 1 μg/ml Pepstatin, 20 mM NaF, phosphatase inhibitor cocktail, 0.5% NP-40 and 10% Glycerol in PBS) and the lysates (1 mg) were cleared by incubation with 50% protein A-Sepharose bead slurry, after which 1 mL of the cleared lysates were incubated with antibody-conjugated 50% protein A-Sepharose beads and 10 μL of 10% BSA overnight at 4° C. The beads were washed three times with washing buffer (0.5% NP-40, 0.1% Triton X-100, 1 mM PMSF, and 1 mM Na₃ VO₄ in PBS). Proteins were revealed after SDS/PAGE and immunoblotting with the indicated antibodies. Immunoblotting analysis of invadopodia proteins fractionated from invasive breast cancer MDA-MB-436 cells confirmed that ASPM interacts with DVL2, PAR6β, CDC42, and N-WASP, but not with other DVL isoforms or SMURF1, in the invadopodia (FIG. 8B).

Having demonstrated that ASPM-i1 specifically interacts with the PAR/PCP complex and its assembly in the invadopodia of cancer cells, we next sought to gain functional insights into its role in the invadopodia biogenesis and cancer invasiveness. To this end, we stably knocked down (knockdown) the expression of the ASPM transcript variant 1 (ASPM-v1) by synthesizing four in silico predicted ASPM-v1 (SEQ ID NO: 3) specific small hairpin RNA (shRNA) oligonucleotides using BLOCK-iT™ RNAi Designer (Invitrogen), which has a 21-nucleotide target sequence spaced within the mRNA region encoded by exon 18 of the ASPM gene (ASPM. e18). We constructed lentiviral vectors expressing each of these ASPM-v1-targeted siRNA by ligating the shRNA sequence containing both sense and antisense strands, separated by a 9-bp loop region for directional cloning into pGLV2-U6-Puro. We infected MDA-MB-436 cells with each of the lentiviral vectors and tested their respective efficacy to downregulate the transcript and the protein abundance levels of ASPM-i1. A non-target siRNA (NT siRNA; SHC002V; Sigma-Aldrich) was used as a control in subsequent experiments.

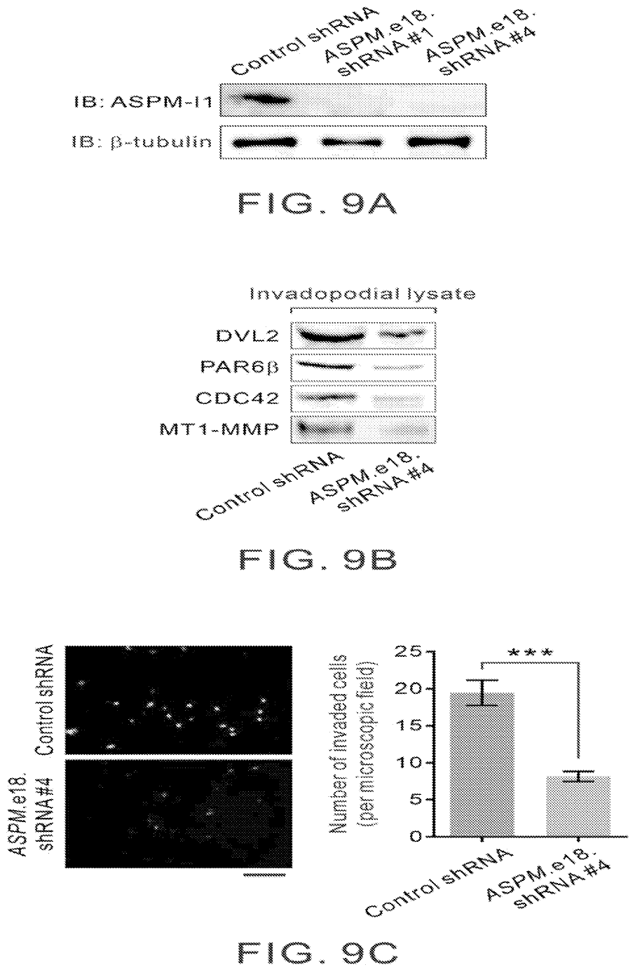

As shown in FIGS. 9A, infection of breast cancer MDA-MB-436 cells with two of the ASPM.c18-specific shRNAs, including ASPM. e18 shRNA #1 and ASPM.c18 shRNA #4, could dramatically downregulate the protein abundance level of ASPM-i1 without affecting that of ASPM isoform 2. Importantly, the specific knockdown of ASPM-v1 expression using the ASPM. e18. #4 shRNA profoundly attenuated the recruitment of the PAR-PCP proteins, including DVL2, PAR6β, and CDC42, and membrane-type matrix metalloproteinase (MTI-MMP; representing the functional protein on invadopodia), to the invadopodia (FIG. 9B), confirming the functional role of ASPM-i1 in the assembly of the invadopodia-specific PAR6-PCP complex. Complementing these molecular analyses, knockdown of ASPM-i1 considerably abrogated the formation of cortactin “F-actin” invadopodia in cancer cells and thereby inhibited their invasive capacity (FIG. 9C). These data collectively suggest that ASPM-i1 regulates invadopodia biogenesis and cancer invasiveness by promoting the assembly of the novel DVL2/PAR6β/CDC42/N-WASP protein complex in invadopodia.

### Example 4 ASPM-i1 Regulates Multiple Development- and Stemness-Associated Pathways in Cancer Cells

ASPM has been identified as a critical regulator of Wnt signaling pathways. ASPM expression was found to be indispensable for cellular responsiveness to canonical Wat ligands, such as Wnt-Ja, in pancreatic and prostate cancer cells. Mechanistic studies revealed that ASPM interacts with upstream activators of β-catenin, including disheveled 2 (DVL2) or DVL3, AXIN, and protease-activated receptor 1 (PAR1) and inhibits the proteasome-dependent degradation of the DVL protein, thereby increasing the protein abundance level of β-catenin and augments canonical Wnt signaling that is important to its oncogenic effect. Consistently, whereas knockdown of ASPM expression resulted in a dramatic decrease in the Wnt reporter activity in cancer cells, elevating DVL expression in these cells could restore their Wnt activity and stemness, implicating DVL can functionally rescue ASPM deficiency and serve as the critical regulator of Wnt signaling-related cancer stemness.

The important role of ASPM in neuron development and its specific expression in normal or malignant stem cells, together with its pleiotropic role in mitotic regulation), cell cycle, and developmental pathways, raising an intriguing possibility of its potential role in other developmental signaling pathways. To this end, we measured the activity of a panel of hallmark development- and stemness-associated pathways using a commercial reporter array (the Cignal Finder Stem Cell & Differentiation 10-Pathway Reporter Array, Qiagen, Germany). Briefly, the highly transfectable human embryonic kidney 293T cells (HEK293T) were knocked downed (knockdown) of their ASPM-v1 expression using lentivirus-mediated shRNA using ASPM. e18. #4. The cells were in-well transfected with the reporter constructs using the Lipofectmine LTX reagent (ThermoFisher Scientific, Waltham, MA, USA), after which the Firefly and Renilla luciferase activity were measured on a SpectraMax® Luminometer (Molecualer Device, Waltham, MA USA) by using the dual luciferase assay system (Promega, Madison, WI, USA). The results were expressed as a ratio of firefly luciferase (Fluc) activity to Renilla luciferase (Rluc) activity.

As shown in FIG. 10, we verified that the Wnt signaling pathway activity significantly decreased in HEK293T cells with knockdown of ASPM-v1 expression. Notably and importantly, the data revealed that knockdown of ASPM-v1 expression also significantly reduced the activities of Notch and Hedgehog (Hh) signaling pathways as well as the stemness-related transcriptional factors OCT4 and KLF4. Together, these data suggest that ASPM-i1 regulates not only Wnt signaling but also other development- and/or stemness-associated pathways in cancer cells, underscoring its critical role in cancer stemness and progression.

### Example 5 in Silico and Experimental Screening of ASPM-v1-Targeted siRNA

To exploit ASPM-i1 as a therapeutic target in the treatment of both cancer growth and cancer aggressiveness via invadopodia formation, we sought to develop siRNA therapy to specifically inhibit the expression of ASPM-i1 in cancer cells. To facilitate the preclinical efficacy test and the primate toxicity studies of ASPM transcript variant 1 (ASPM-v1; NCBI RefSeq: NM_018136.4; encoding ASPM-i1)-targeted siRNA therapy, we searched siRNA sequences that simultaneously target the mRNA region that is encoded by exon 18 of the ASPM gene (unique to ASPM-v1; “ASPM-v1. e18”) of both human ASPM-v1 and that of cynomolgus monkey (Macaca fascicularis). We aimed at designing siRNA that target different regions on the mRNA of exon 18 of ASPM-v1; we evenly divided exon 18 of human ASPM-v1 into four segments of similar lengths and aligned them with the corresponding regions on the cynomolgus monkey gene using the NCBI BLAST search. A high identity (96%-98%) was found between human and primate sequences for each of these segments (Table 1).

TABLE 1
The four highly homologous mRNA regions encoded by
exon 18 of human and cynomolgus monkey ASPM genes

| | | Nucleotide | Nucleotide | |
|---|---|---|---|---|
| | | location (start) | location (end) | Identify of |
| Fragment | Species | on ASPM-v1 | on ASPM-v1 | alignment |
| ASPM-v1.e18.F1 | Homo sapiens | 4066 | 5253 | 97% |
| | Cynomolgus monkey | 4066 | 5253 | |
| ASPM-v1.e18.F2 | Homo sapiens | 5254 | 6441 | 97% |
| | Cynomolgus monkey | 5254 | 6441 | |
| ASPM-v1.e18.F3 | Homo sapiens | 6442 | 7629 | 96% |
| | Cynomolgus monkey | 6442 | 7629 | |
| ASPM-v1.e18.F4 | Homo sapiens | 7630 | 8820 | 98% |
| | Cynomolgus monkey | 7630 | 8820 | |

We then searched the various identical sub-segments (data not shown) located within each of the four ASPM-v1. e18 segments and designed siRNA for each of these sub-segments based on the siRNA Selection Server at the Whitehead Institute for Biomedical Research (http://sima.wi.mit. edu/home.php). We applied the following three selection criteria to select the candidate siRNA: (1) a GC content of 40-60%, (2) a negative difference between the binding energies of 5′ end sense strand and the 5′ end antisense strand (ΔΔG), (3) an off-target score greater than 10. The off-target score of a siRNA was determined by subjecting said siRNA to a bomology search against human mRNA sequences using NCBI BLAST, followed by extracting the numbers of mismatches in the non-seed region, the seed region, as well as the cleavage site region on said siRNA for the calculation of the score, as described previously (U.S. Pat. No. 8,809,292 B2), and (4) the lack of known immunostimulatory motifs, including (5′ to 3′) “GUCCUUCAA”, and “UGUGU” (Fedorov et al., 2006; Judge et al., 2005). Using these criteria, we identified 42 candidate siRNA that fulfill these selection criteria (Step 1). To verify the gene-silencing of the knockdown (knockdown) efficacy of the ASPM-v1-targeted siRNAs (siASPM-v1), we synthesized the double-stranded siRNA (Dharmacon) corresponding to the selected sequences and transfected each of them into the highly tranducible human embryonic kidney HEK293T cells using the Lipofectamine LTX Reagent (ThermoFisher Scientific), A siRNA (siASPM-v1.8602; 5′-GAGCUGCUAUCACUUUACAGC-3″), which had been previously validated for its knockdown effect on ASPM-v1 expression, was also synthesized and included as a positive control (Hsu et al., 2019b). A non-target control siRNA (5′-UGGUUUACAUGUCGACUAAUU-3′; Dharmacon) was synthesized and included as a negative control. We measured the transcript level of ASPM-v1 using quantitative real-time PCR (qRT-PCR) using the LightCycler FastStart DNA MASTERPLUS SYBR Green I Kit and the LightCycler System (Roche Diagnostics GmbH, Mannheim, Germany). Oligonucleotide primers were designed using Primer Bank (http://pga.mgh.harvard. edu/primerbank/index.html). All the transduction experiments were performed in triplicate to allow for statistical analyses. We ranked the 42 candidate siRNAs according to their respective gene-silencing effect on the transcript level of ASPM-v1. Of note, not all these in silico designed siASPM-v1 could effectively knockdown the expression of ASPM-v1, with three of them not affecting the transcript level of ASPM-v1 after being transduced into HEK293 cells. Using the Student's t-test, we identified a list of 30 of siRNAs from these 42 candidate siRNAs that could significantly (P value less than 0.05) reduce the expression of ASPM-v1 when transduced into HEK293K cells.

To verify the knock-down (knockdown) effect of the siRNAs designed above, we synthesized the double-stranded siRNAs (Dharmacon) corresponding to the selected sequences and transfected each of them into the highly tranducible human embryonic kidney HEK293T cells using the Lipofectamine LTX Reagent (ThermoFisher Scientific). A siRNA (siASPM-v1.8602; 5′-GAGCUGCUAUCACUUUACAGC-3′), which had been previously validated for its gene-silencing effect on ASPM-v1 expression, was also synthesized and included as a positive control (Hsu, C. C., Liao, W. Y., Chan, T. S., Chen, W. Y., Lee, C. T., Shan. Y. S., Huang, P. J., Hou, Y. C., Li. C. R., and Tsai, K. K. (2019b). The differential distributions of ASPM isoforms and their roles in Wnt signaling, cell cycle progression, and pancreatic cancer prognosis. J Pathol). A non-target control siRNA (NT siRNA; 5′-UGGUUUACAUGUCGACUAAUU-3′; Dharmacon) was synthesized and included as a negative control. We measured the transcript level of ASPM-v1 using quantitative real-time PCR (qRT-PCR) using the LightCycler FastStart DNA MASTERPLUS SYBR Green 1 Kit and the LightCycler System (Roche Diagnostics GmbH, Mannheim, Germany), Oligonucleotide primers were designed using Primer Bank (http://pga.mgh.harvard. edu/primerbank/index.html). We then ranked the 45 candidate siRNAs according to their respective knockdown effect on the transcript level of ASPM-v1 and then selected the two top-ranked siRNA sequences from each of the four human-monkey homogeneous fragments as shown in Table 1 (Step 2). To further select the top-ranked siRNAs, we transduced breast cancer MDA-MB-436 cells with the eight siRNAs selected from Step 2 and ranked them according to their respective knockdown effect (Step 3). We then selected the three top-ranked siRNA sequences for the subsequent analysis, which include siASPM-v1.F2 #1, siASPM-v1. F3 #1, and siASPM-v1. F4 #1 (Step 4).

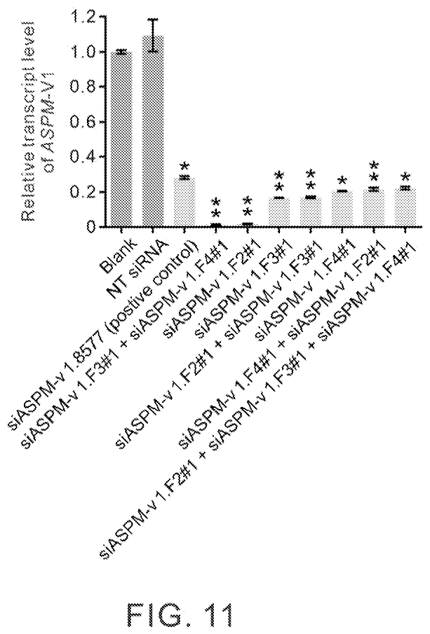

To compare the efficacy of each of the three selected siRNAs or their combinations to knock down the expression of ASPM-v1, we transduced MDA-MB-436 cells with each of the three siRNAs or their different combinations and analyzed the respective knockdown effect using qRT-PCR. Interestingly, we found the 1:1 mixture of two of the siRNAs, including siASPM-v1. F3 #1 and siASPM-v1. F4 #1, as well as the single siRNA siASPM-v1. F2 #1, achieved the best gene-silencing effect on ASPM-v1 among the different combinations (FIG. 11). Notably and importantly, the 1:1 mixture of siASPM-v1. F3 #1 and siASPM-v1. F4 #1 was chosen over the single siASPM-v1. F2 #1 for subsequent developments due to the following considerations: (1) the target mRNA sequences of siASPM-v1. F3 #1 and siASPM-v1. F4 #1 are located at different fragments on exon 18 of the ASPM gene (i.e., fragments 3 and 4, respectively) and therefore their simultaneous targeting using the siRNA mixture may obviate the potential variations in the knockdown efficacy caused by chromatin conformations, and (2) the siRNA mixture consists of each siRNA at an amount half of that in a single siRNA, whereby the potential toxicity, such as off-target silencing and immune-stimulatory effects, can be theoretically mitigated.

### Example 6 Biological Effects of the Lipid-Nanoparticle-Formulated ASPM-v1-Targeted siRNA on Cancer Cells In Vitro

Many types of solid human cancers, such as breast cancer, non-small-cell lung carcinoma, pancreatic ductal adenocarcinoma (PDAC), the scirrhous subtype of gastric adenocarcinoma, and the “stem/serrated/mesenchymal” molecular subtype of colorectal cancer, are characterized by a pronounced stromal reaction termed “the desmoplastic response”, which constitutes a major obstacle for the efficient transport of cancer therapeutics into the tumor. Recently, two nanoparticle-formulated chemotherapy, including albumin-bound paclitaxel (nab-paclitaxel) and liposome-encapsulated irinotecan, have been shown to extend the survival of patients with advanced PDAC. Both reagents could significantly increase the levels of the chemotherapeutic agents in the treated tumors, suggesting that nanoparticle formulation is a clinically validated approach to improve the treatment efficacy of desmoplastic cancers. In liver disease, parenterally administration of a liposome-encapsulated siRNA specific for transthyretin (Patisiran, Alnylam) has been shown to reduce up to 86.8% of transthyretin produced by the liver in patients with hereditary transthyretin-mediated amyloidosis and thus became the first clinically approved RNAi drug (Adams, D., Gonzalez-Duarte, A., O'Riordan, W. D., Yang, C. C., Ueda, M., Kristen, A. V., Tournev, L., Schmidt, H. H., Coelho. T., Berk. J. L., et al. (2018). Patisiran, an RNAI Therapeutic, for Hereditary Transthyretin Amyloidosis. N Engl J Med 379, 11-21). Moreover, nanoparticle-delivered siRNA therapy, such as cyclodextrin polymer-based nanoparticles carrying siRNA targeting ribonucleotide reductase M2 (RRM2) and lipid nanoparticles carrying siRNA targeting VEGF-A and kinesin spindle protein (KSP), have shown promising pharmacodynamics and tolerability and anti-tumor efficacy in some of the treated patients in phase 1 clinical trials (Davis, M. E., Zuckerman, J. E., Choi, C. H., Seligson, D., Tolcher, A., Alabi, C. A., Yen, Y., Heidel, J. D., and Ribas, A. (2010). Evidence of RNAi in humans from systemically administered siRNA via targeted nanoparticles. Nature 464, 1067-1070). Recently, intravenous injections of a liposomal nanoparticle (LNP)-encapsulated small activating RNA designed to activate the transcription of the CEBPA gene has shown an acceptable safety profile and potential synergistic efficacy with tyrosine kinase inhibitors in a phase 1b study of patients with advanced hepatocellular carcinoma (Sarker, D)., Sudergren, M., Plummer. E. R., Basu, B., Meyer, T., Huang, K. W., Evans, T. R. J., Spalding, D., Ma, Y. T., Palmer, D. H., et al. (2020). First-in-human phase I trial of small activating RNA (saRNA) oligonucleotide MTL-CEBPA in combination with sorafentib in patients with advanced hepatocellular carcinoma (HCC). Journal of Clinical Oncology 38). These clinical advances, together with the crucial role of ASPM-i1 in the invadopodia biogenesis and cancer invasiveness, incited us to develop a nanoparticle-formulated and ASPM-v1-targeted siRNA gene therapy in the treatment of invasive and metastatic cancers.

Unmodified siRNA is vulnerable to serum exo- and endo-nucleases, leading to a short half-life in serum, and can induce immune responses via interferons and proinflammatory cytokines (Watts, J. K., Deleavey, G. F., and Damha, M. J. (2008). Chemically modified siRNA: tools and applications. Drug Discov Today 13, 842-855). Therefore, a number of chemical modifications have been explored to improve the stability of siRNA and to render siRNA less immunogenic (Hassler, M. R., Turanov, A. A., Altermon, J. F., Haraszti, R. A., Coles, A. H., Osborn, M. F., Echeverria, D., Nikan, M., Salomon, W. E., Roux, L., et al. (2018). Comparison of partially and fully chemically-modified siRNA in conjugate-mediated delivery in vivo. Nucleic Acids Res 46, 2185-2196). To avoid potential immunogenicity of these siRNA and to increase their stability in serum, we added the 2′-O-methylation modification at site UA or CA in the antisense strand and at all pyrimidine in the sense strand since the 2′-O-methylation modification of siRNA of the selected siRNA, which has been associated with less immune activation when administered systematically (Adami, R. C., Seth, S., Harvie, P., Johns, R., Fam, R., Fosnaugh, K., Zhu, T., Farber, K., Mccutcheon, M., Goodman, T. T., et al. (2011). An amino acid-based amphoterie liposomal delivery system for systemic administration of siRNA. Mol Ther 19, 1141-1151). To enhance the serum stability, we additionally added two deoxy-thymidine 3′ overhangs with phosphorothioate linkage to both the sense and the antisense strand.

The double-stranded and partially chemically modified siASPM-v1.F3 #1, siASPM-v1.F4 #1, and the similarly modified non-target double-stranded oligonucleotide (Sense strand: 5′-UGGUUUACAUGUCGACUAA-3′; non-target control siRNA) were synthesized by Dharmacon (Horizon Discovery Ltd., Waterbeach, UK). A UU dinucleotide is added to the 3′ end of both oligonucleotide strands as overhangs. Each of them was formulated with a lipid nanoparticle (LNP)-based delivery vehicle comprising the ionizable cationic amino lipid DLin-MC3-DMA (MC3), which complexes with siRNA, the amphipathic phospholipid distcaroyl-phophatidylcholine (DSPC), cholesterol and a coat lipid poly(ethylene glycol) lipid 1,2-dimyristoyl-rac-glycerol-methoxy (poly(ethylene glycol) (DMG-PEG) mixed at the molar ratio of 50:10:38.5:1.5 (Adams, D., Gonzalez-Duarte, A., O'Riordan, W. D., Yang, G. C., Veda, M., Kristen, A. V., Tournev, 1., Schmidt, H. H., Coelho, T., Berk, J. L., et al. (2018). Patisiran, an RNAi Therapeutic, for Hereditary Transthyretin Amyloidosis. N Engl J Med 379, 11-21). The rationales for choosing this MC3-based LNP as the delivery vehicle of our ASPM-targeted siRNA therapy are three-fold. First, the same LNP formation is used in the first FDA-approved siRNA drug patisiran (Onpattro) developed by Alnylam Pharmaceuticals. Second, this LNP formulation has been used for systemic delivery of siRNA targeting tumor-driving genes such as BCR-ABL, VEGF-A, and kinesin spindle protein (KSP), which showed significant therapeutic efficacy in a mouse model of chronic myeloid leukemia (CML) (Jyotsana, N., Sharma, A., Chaturvedi, A., Budida, R., Scherr, M., Kuchenbauer, F., Lindner, R., Noyan, F., Suhs, K. W., Stangel, M., et al. (2019). Lipid nanoparticle-mediated siRNA delivery for safe targeting of human CML in vivo. Ann Hematol 98, 1905-1918) or an orthotopic murine model of hepatocellular carcinoma (HCC) (Tabernero, J., Shapiro, G. I., LoRusso, P. M., Cervantes, A., Schwartz, G. K., Weiss, G. J., Paz-Ares, L., Cho, D. C., Infante, J. R., Alsina, M., et al. (2013). First-in-humans trial of an RNA interference therapeutic targeting VEGF and KSP in cancer patients with liver involvement. Cancer Discov 3, 406-417). Third, systemic delivery of siRNA based on this LNP formation was shown to be safe and generally well-tolerated in phase 1 clinical trial except for some infusion-related reactions and transient proinflammatory cytokine induction. The particle size of the MC3-based LNP is in the range of 70-90 nm (Jayaraman et al., 2012), which is associated with an extended circulation time and permits its leakage into the tumor tissues through the leaky endothelial fenestrations (with estimated pore sizes of 380-780 nm), a phenomenon known as the “enhanced permeability and retention (EPR)” effect (Agarwal and Roy, 2013; Jain and Stylianopoulos, 2010).

We synthesized DLin-MC3-DMA and purchased DSPC (Avanti Polar Lipids, Alabaster, AL, USA), cholesterol (Sigma-Aldrich), and DMG-PEG (NOF America Corporation, White Plains, NY, USA). We encapsulated the chemically modified formulate the above-mentioned mixture of siASPM-v1. F3. #1 and siASPM-v1. F4. #1 an in-house designed microfluidic apparatus for controlled mixing conditions at a flow rate of 0.5 ml/min and a flow rate ratio of 1:3 (lipid:siRNA=0,125 ml/min: 0.375 ml/min). The size (number-weighted mean diameter) and ζ-potential of the LNPs were measured by a Zetasizer Nano ZS ZEN3600 instrument (Malvern Instruments. Worcestershire, UK). The encapsulation efficiency and total concentration of siRNA were measured using the Quanti-iTIM RiboGreen RNA Reagent and Kit (Invitrogen, Waltham, MA, USA). The resultant MC3-based LNP-capsulated 1:1 mixture of chemically modified siASPM-v1. F3 #1 and siASPM-v1. F4 #1 was designated as “LNP-siASPM-v1”.

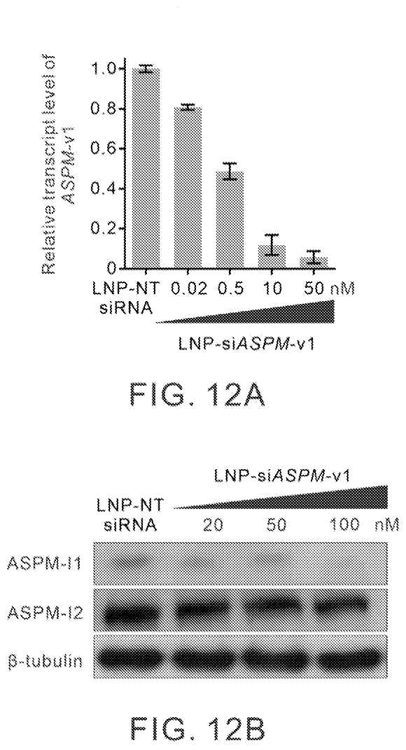

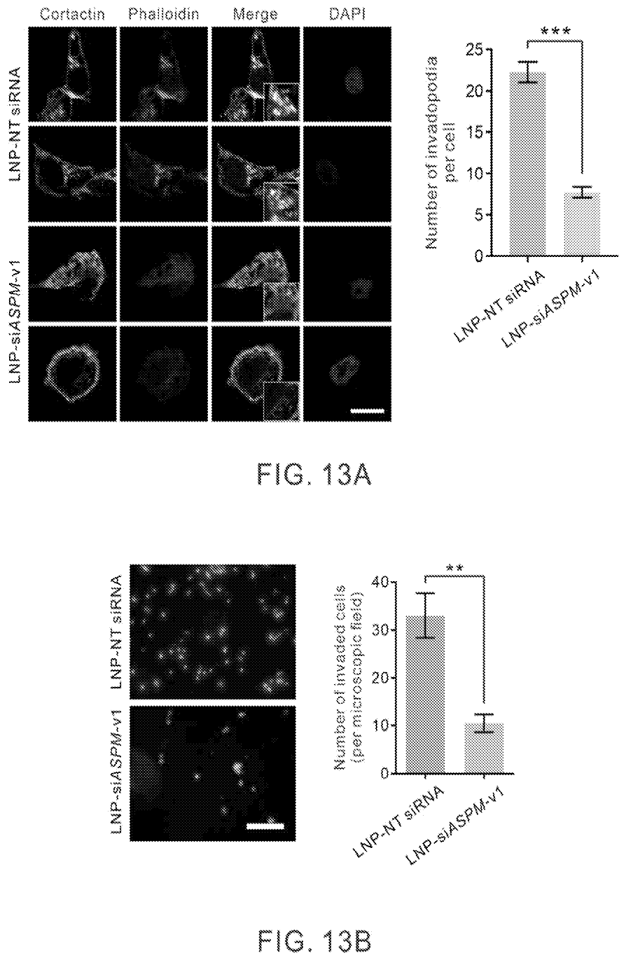

We first verified that treatment of MDA-MB-436 cells with LNP-siASPM-v1 could reduce the transcript level of ASPM-v1 in a dose-dependent manner with a 50% inhibitory concentration (IC50) of 0.43 nM (FIG. 12A). Consistently, Immunoblotting analysis confirmed that the treatment dose-dependently reduced the protein abundance level of ASPM-i1 (FIG. 12B). By contrast, the treatment did not affect the transcript level of ASPM transcript variant 2 (data not shown) or the protein abundance level of ASPM-i2 (FIG. 12B), reaffirming its specific knockdown effect on ASPM-v1 expression. In accordance with the effect of knockdown on ASPM-i1, treatment of cancer cells with siASPM-v1 considerably abrogated the formation of cortactin “F-actin” invadopodia (FIG. 13A) as well as their invasive capacity (FIG. 13B).

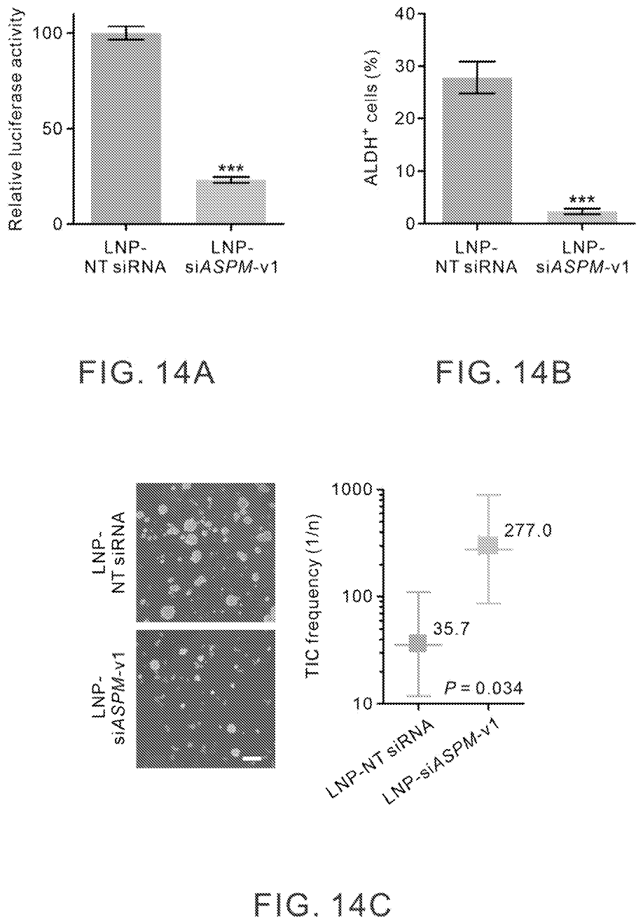

Previously, ASPM, specifically ASPM-i1, has been shown to contribute to canonical Wat activity, stemness and tumorigenesis in various types of cancers, such as pancreatic cancer, prostate cancer, and hepatocellular carcinoma (HCC) (Hsu, C. C., Liso, W. Y., Chan, T. S., Chen, W. Y., Lee, C. T., Shan, Y. S., Huang, P. J., Hou, Y. C., Li, C. R., and Tsai, K. K. (2019a). The differential distributions of ASPM isoforms and their roles in Wnt signaling, cell cycle progression, and pancreatic cancer prognosis. J Pathol 249, 498-508). We, therefore, sought to evaluate the effect of the ASPM-v1-targeted siRNA on the Wnt activity and stemness of cancer cells. To this end, we treated HCC HuH-1 cells with the LNP-siASPM-v1 and verified that it could substantially reduce WNT3A-stimulated Wnt-specific TEF/LEF reporter activity in HuH-1 cells (FIG. 14A). Consistently, it could also diminish the population of aldehyde dehydrogenase (ALDH)-positive cells, which are known to contain the enriched stem-like cells in HCC (FIG. 14B). At the functional level, the transduction of HuH-1 cells with LNP-ASPM-v1 siRNA could substantially inhibit their ability to form tumorspheres in a serum-free and ultra-low attachment culture condition (FIG. 14C). These findings collectively support the Wnt- and stemness-inhibitory functions of LNP-formulated ASPM-v1-targeted siRNA.

### Example 7 Pharmacodynamic Studies of the Lipid-Nanoparticle-Formulated ASPM-v1-Targeted siRNA Therapy

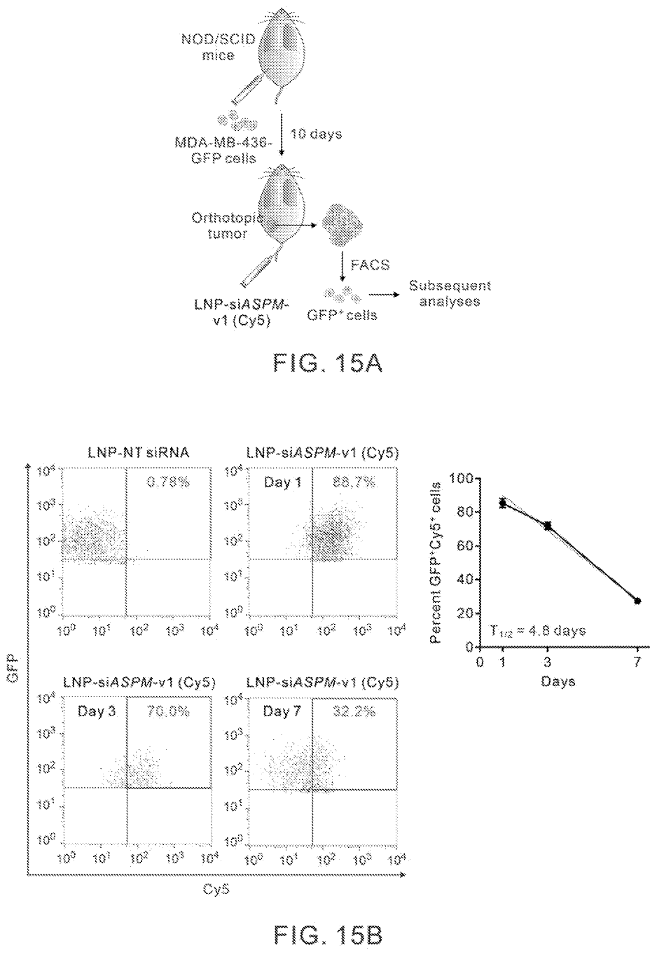

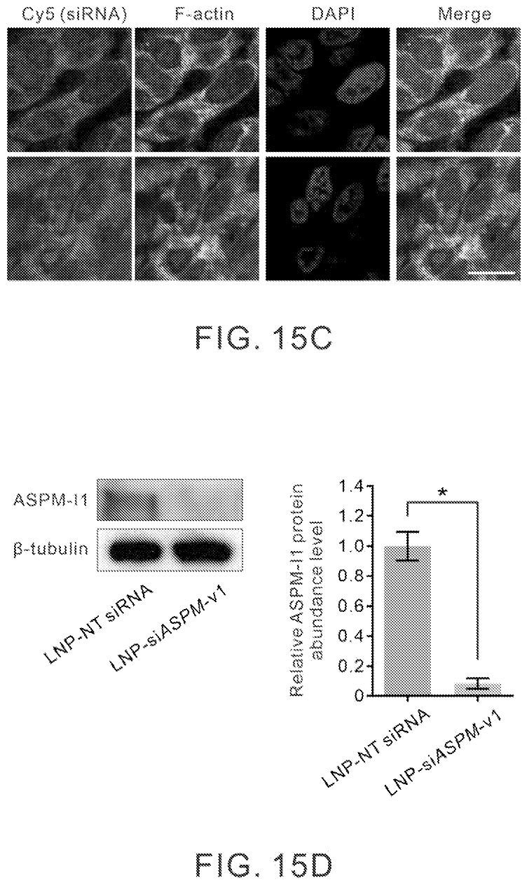

To ascertain the pharmacodynamic activity of the systemic LNP-siASPM-v1 therapy in treated tumor cells, triple-negative breast cancer (TNBC) MDA-MB-436 cells were lentivirally transduced a GFP and firefly luciferase (FF-Luc) fusion vector (UBC-EGFP-T2A-Luc; System Biosciences) and GFP-positive cells were sorted using the BD Influx™ Cell Sorter (BD Biosciences). The cells were then injected orthotopically into the mammary fat pads of immunodeficient NOD/SCID mice. Ten days following cell inoculation, when the tumors were detectable by bioluminescence (BLI), the tumor-bearing mice received intravenous (IV) injections of LNP-siASPM-v1 at the dose of 100 μg per mouse (approximately 4 mg/kg). The treatment was repeated three days later, and the tumors were removed 24 hours following the second injection. The tumors were enzymatically dissociated into single cells, and the GFP⁺ cancer cells were sorted using FACSAria™ III cell sorter (BD) Biosciences) for the subsequent analysis (FIG. 15A).

As shown in FIGS. 15B, the majority (88.7% on average) of the GFP⁺ humor cells isolated from the LNP-siASPM-v1-treated mice were Cy5-positive 24 hours following completion of the treatment, affirming that they were successfully transduced with the siRNA. The percentage of cells transduced with siRNA gradually decreased over time, with an estimated half-life of 4.8 days.

Next, to ascertain the accumulation of LNP-siASPM-v1 in tumor cells, the tumor tissues removed from the siRNA-treated mice were snap-frozen in liquid nitrogen and sectioned for immunofluorescence staining with Phalloidin (stain for actin filaments) to mark the cell contour. As shown in FIGS. 15C, confocal imaging revealed that the systemically administrated siRNA was successfully delivered to the cytoplasm of the tumor cells in the orthotopic TNBC model.

Finally, to ascertain the knockdown effect of the systemic LNP-siASPM-v1 therapy on the protein abundance level of ASPM-i1 in the treated tumor cells, the protein lysates were collected from the GFP tumor cells freshly sorted from the treated tumors and blotted with an ASPM-i1-specific rabbit polyclonal antibody (Hsu et al., 2019a). As anticipated, the siRNA therapy substantially reduced the protein abundance level of ASPM-i1 compared to that of the untreated tumor FIGS. 15D, affirming that the systemic siASPM-v1 therapy does specifically inhibit the expression of ASPM-i1 in the treated tumor cells.

### Example 8 Anti-Tumor and Anti-Metastasis Effects of the ASPM-v1-Targeted siRNA Therapy in Triple-Negative Breast Cancer (TNBC)

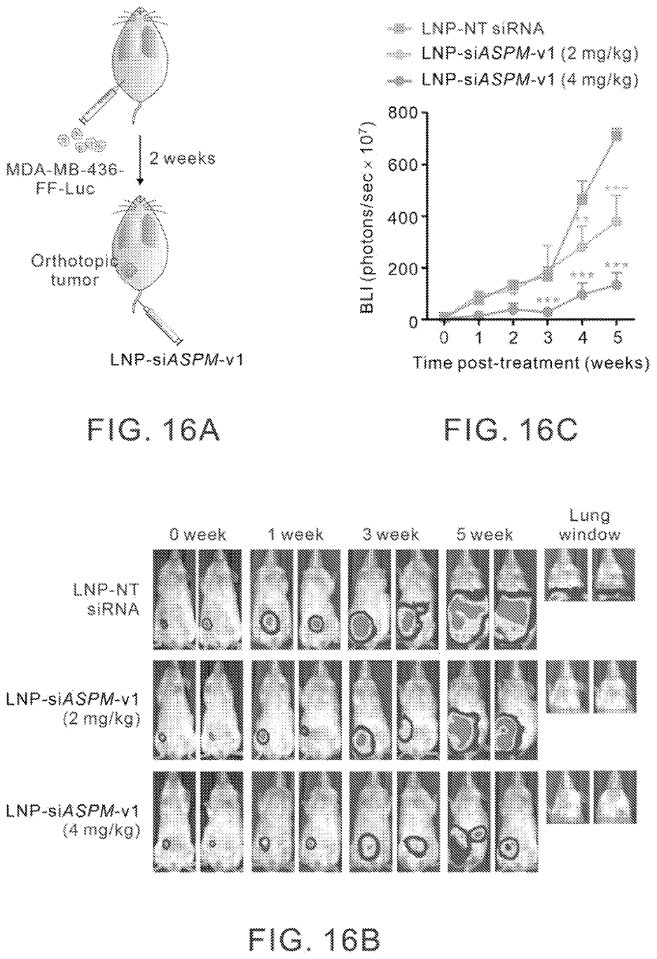

Having demonstrated the anticipated inhibitory effects of LNP-ASPM-v1 siRNA on the invadopodia formation and the invasive capacity of cancer cells, we next sought to investigate its therapeutic effect in vivo. We explored if the systemic LNP-siASPM-v1 could exert anti-metastasis efficacy using an orthotopic breast cancer progression model. The GFP- and FF-Loc-expressing TNBC MDA-MB-436 cells (10⁶ cells) established in Example 7 were injected orthotopically into the mammary fat pads of NOD/SCID mice. Ten days following cell inoculation, when the tumors were detectable by BLI, the tumor-bearing mice received repetitive intravenous (IV: 100 μg per mouse [approximately 4 mg/kg] every 3 days; 10 doses in total) injections of LNP-siASPM-v1 or LNP-non-target control siRNA (NT siRNA). The resultant primary or metastatic pulmonary tumors were visualized by bioluminescence imaging (BLI) weekly according to the manufacturer's recommendations (IVIS Imaging System, Caliper Life Sciences) (FIG. 16A). The results showed that IV LNP-siASPM-v1 therapy at different dose levels (2 mg/kg or 4 mg/kg per injection) could significantly reduce the growth of the primary tumors in a dose-dependent manner with a tumor-control rate of 81.2% at the high-dose level (FIG. 16B). As expected, the systemic gene therapy could completely prevent the occurrence of distant metastatic tumors in the lung (FIG. 16C), highlighting the strong anti-tumor and anti-metastasis efficacy of the ASPM-v1-targeted siRNA therapy.

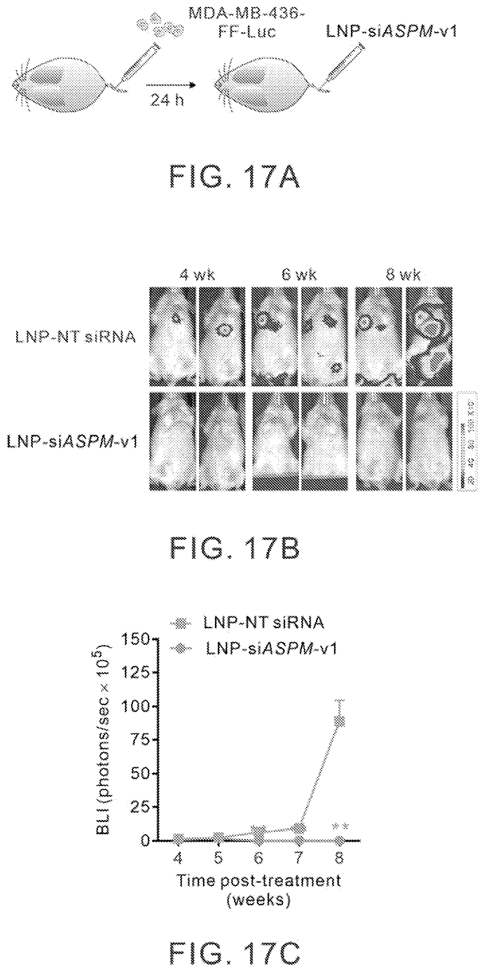

Despite the advances in adjuvant chemotherapy, approximately 40% of patients with breast cancer who receive surgical removal of their primary tumors develop recurrence and ultimately die of metastatic diseases (Weigelt, B., Peterse, J. L., and van't Veer, L. J. (2005). Breast cancer metastasis: markers and models. Nat Rev Cancer 5, 591-602). The current adjuvant chemotherapy only increases the 15-year survival of breast cancer patients by 3%-10%, highlighting the pressing need to develop anti-metastasis therapies that are safer and more efficacious than chemotherapy. Along this line, the ability of nanoparticles to improve the tissue penetration and tumor uptake of the cargo drugs, together with their potential to combine multiple therapeutic functions into a single platform, position nanoparticle therapeutics as a highly promising strategy in the treatment of metastatic cancers (Schroeder, A., Heller, D. A., Winslow, M. M., Dahlman, J. E., Pratt, G. W., Langer, R., Jacks. T., and Anderson, D. G. (2011). Treating metastatic cancer with nanotechnology. Nat Rev Cancer 12, 39-50). Since the ASPM-v1-targeted siRNA therapy shows dramatic anti-metastasis efficacy in orthotopic models of breast cancer, we posited that this nano-gene therapy will also be of great utility in the prevention of metastatic disease. To address this possibility, we injected MDA-MB-436 cells through tail veins in NOD/SCID mice. Twenty-four hours after cell inoculation, the mice received IV injections of LNP-siASPM-v1 (4 mg/kg per mouse every 3 days; 6 doses in total) (FIG. 17A). Indeed, the therapy completely prevented the occurrence of distant metastasis, especially in the lung (FIG. 17B and FIGS. 17C), supporting the therapeutic potential of the ASPM-v1-targeted siRNA therapy at the adjuvant setting.

### Example 9 Anti-Tumor Efficacy of the ASPM-v1-Targeted siRNA Therapy in Hepatocellular Carcinoma (HCC)

Our preceding findings demonstrated that the ASPM-v1-targeted siRNA therapy inhibited the invadopodia formation and the invasiveness of cancer cells as well as their Wnt activity and steniness properties (FIG. 14). Consistently, intratumoral (IT) injections of the LNP-siASPM-v1 could reduce the bulk of primary tumors in the aforementioned orthotopic breast cancer model. These findings, together with the recent FDA approval of the fiposame-encapsulated siRNA therapy for the liver disease transthyretin-mediated amyloidosis (Patisiran, Alnylam) as well as the crucial role of the ASPM gene in HCC tumorigenicity and progression, incited us to evaluate the therapeutic potential of ASPM-v1-targeted siRNA therapy in the treatment of local and/or advanced HCC. We thus administered LNP-siASPM-v1 into established HCC through IT injections in a subcutaneous xenograft mouse model with or without the concurrent oral administration of sorafenib, a multi-kinase inhibitor routinely used in the first-line treatment of advanced HCC that also has a Wnt-inhibitory effect (Lachenmayer, A., Alsinet, C., Savic, R., Cabellos, L., Toffanin, S., Hoshida, Y., Villanueva, A., Minguez, B., Newell, P., Twar, H. W., et al. (2012). Wnt-pathway activation in two molecular classes of hepatocellular carcinoma and experimental modulation by sorafenib. Clin Cancer Res 18, 4997-5007). In this model, HuH-1 cells were lentivirally transduced with a GFP and firefly luciferase fusion vector, and the GFP-positive cells were sorted as described above. The cells (1×10⁶ cells) in 100 μl (1:1 Matrigel:cells) were inoculated subcutaneously into the flanks or orthotopically into the left hepatic lobe of 8-week-old NOD/SCID mice, and the tumor mass and distribution were assessed by BLI.

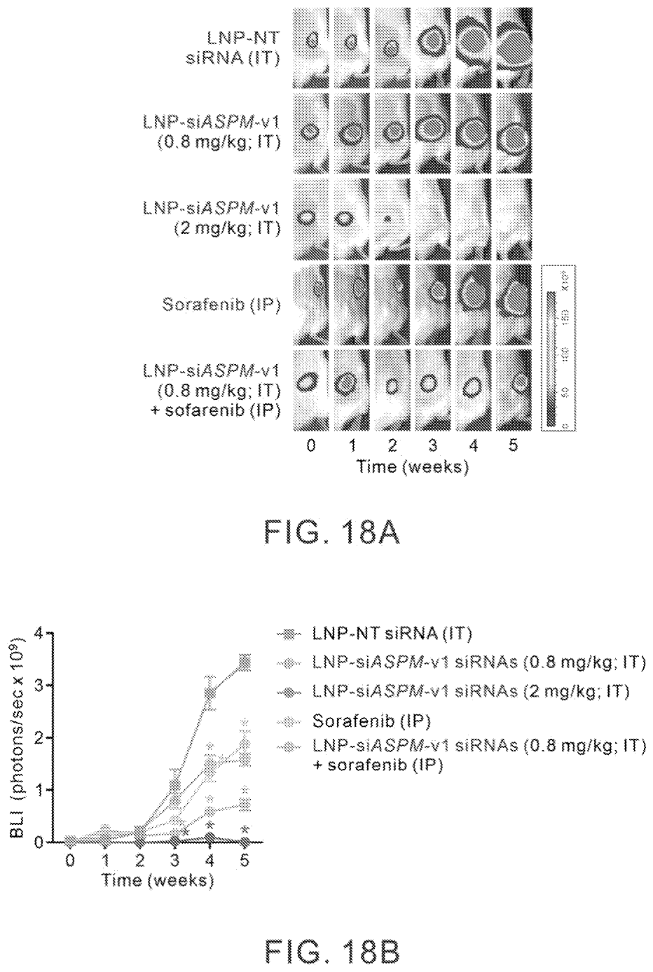

As shown in FIGS. 18A, intra-tumoral injections of LNP-siASPM-v1 (0.8 mg/kg or 2 mg/kg every 3 days for 3 doses in total) significantly attenuated tumor growth in a dose-dependent manner. Remarkably, the treatment dramatically reduced primary tumor growth with an average tumor-control rate of 92.8% at the 2 mg/kg dose level. While the LNP-siASPM-v1 therapy at the 0.8 mg/kg dose level had less significant anti-tumor efficacy, it could markedly enhance the anti-tumor efficacy of the standard HCC therapeutic agent sorafenib, implicating the potential synergistic effect of the LNP-formulated ASPM-v1-targeted siRNA with sorafenib in the treatment of HCC (FIG. 18B).

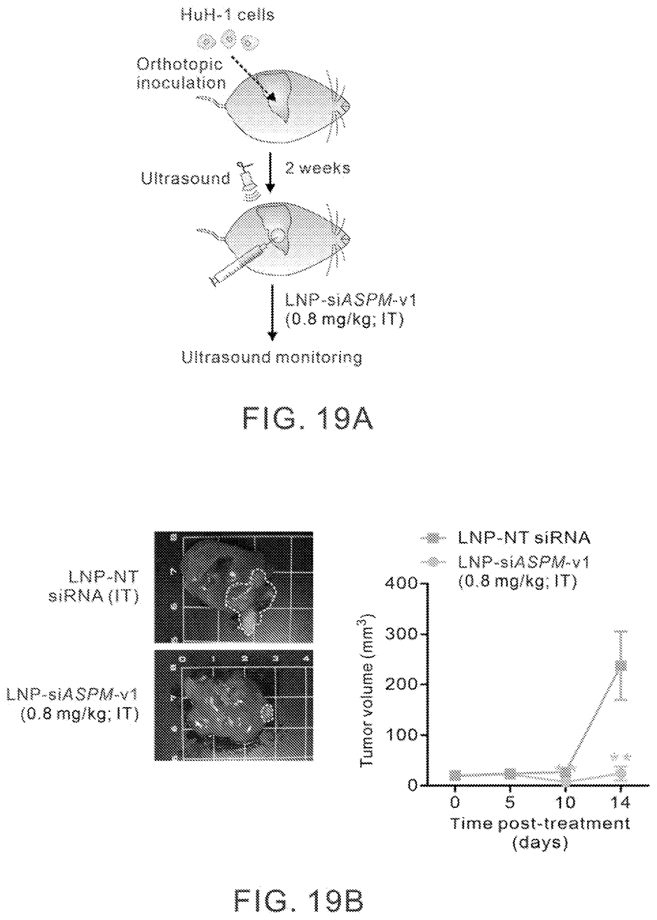

Next, we sought to further extend our findings to a more clinically relevant context. Since image-guided local treatments such as ablation and transcatheter tumor therapy are widely accepted treatment options for patients with early-stage HCC, we conducted ultrasound-guided injections of the LNP-formulated ASPM-v1-targeted siRNA in an orthotopic mouse model of HCC (FIG. 19A). Under the guidance of ultrasound, human HCC HuH-1 cells were injected into the left lobe of the livers of NOD/SCID mice. Three weeks following cell inoculation, LNP-siASPM-v1 was injected directly into the tumors under the guidance of ultrasound. Remarkably, injections of LNP-siASPM-v1 at a low-dose level (0.8 mg/kg) into the orthotopic HuH-1 tumors could substantially attenuate their growth (FIG. 19B), supporting the intratumoral ASPM-v1-targeted siRNA therapy as a clinically feasible way to treat patients with early-stage HCC presenting with approachable lesions.

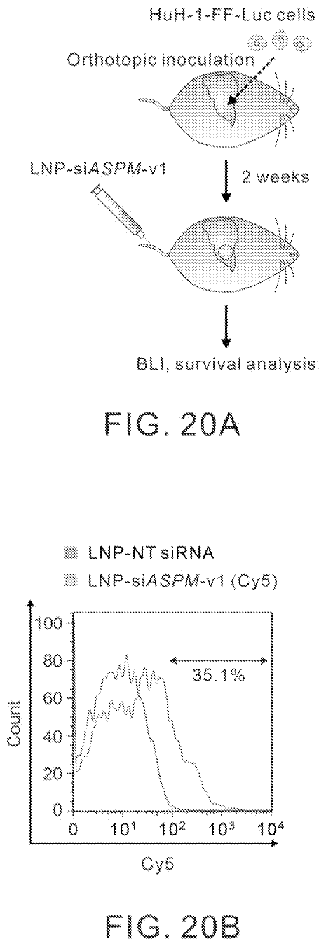

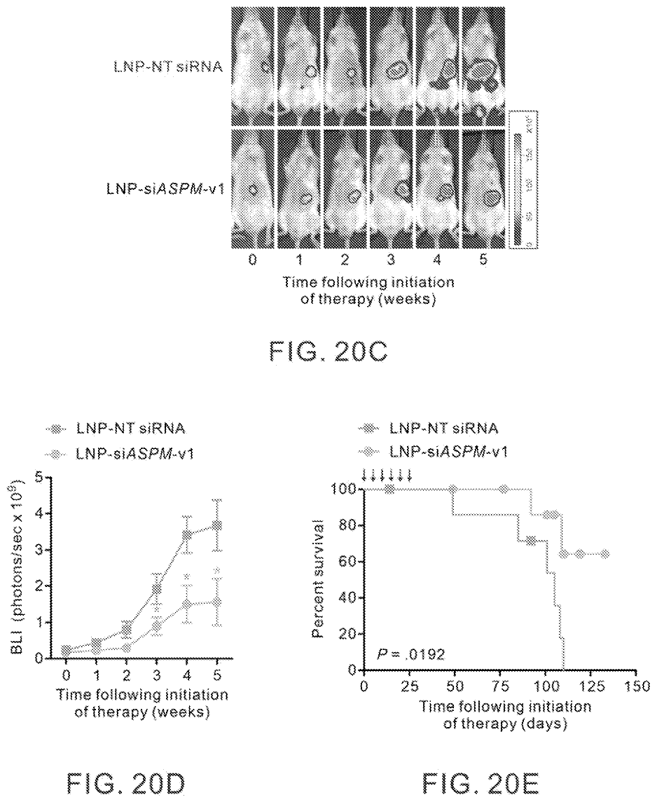

Systemic administration of multi-kinase inhibitors or immune checkpoint inhibitors is the treatment options for patients with advanced HCC, whereas they are associated with a very limited survival benefit or response rate. We thus investigated if systemic administrations of the LNP-formulated ASPM-v1-targeted siRNA also exert anti-tumor efficacy in an orthotopic model of HCC. Briefly, FF-Luc-expressing HuH-1 cells (1×10⁶ cells) in 100 μl (1:1 Matrigel:cells) were inoculated subcutaneously into the flanks or orthotopically into the left hepatic lobe of 8-week-old NOD/SCID mice. Two weeks following tumor establishment, LNP-siASPM-v1 (100 μg per mouse [approximately 4 mg/kg] every 3 days; 6 doses in total) or LNP-non-target control siRNA was administered through tail vein injections (FIG. 20A). As shown in FIGS. 20B, systemically administered LNP-siASPM-v1 could efficiently transduce tumor cells in the orthotopically established HCC. Importantly, the systemic LNP-siASPM-v1 therapy was able to markedly attenuate tumor progression with an average tumor-control rate of 57.5% (FIG. 20C and FIG. 20D); therefore, the mice receiving the therapy survived significantly longer than those receiving the control treatments (FIG. 20E).

### Example 10 a Combination of siRNAs that Effectively Downregulate the Expression of ASPM-v1 in Various Types of Human Cancer Cells

Our preceding findings in Example 5 have shown that the gene-silencing effect of the ASPM-v1-specific siRNAs varied considerably among different types of malignant cells, such as HEK293T cells and breast cancer MDA-MB-436 cells (FIG. 11). Since we sought to select siRNAs that can effectively silence the expression ASPM-v1 in various types of cancers, we undertook to re-select siRNAs that can achieve the best knockdown efficacy across different malignant cells.

To this end, we first selected ten siRNAs that could achieve a more than 80% of knockdown effect on the expression of ASPM-v1 in HEK293T cells from the thirty siRNAs described in Example 5. We then acquired the synthesized double-stranded siRNAs (Dharmacon) and transduced each of them into HEK293T cells, the TNBC line MDA-MB-436 cells, and the HCC line HuH-1 cells using Lipofectamine LTX Reagent (ThermoFisher Scientific). A SÍRNA (siASPM-v1.8602; 5′-GAGCUGCUAUCACUUUACAGC-3′) that had been previously validated for its knockdown effect on ASPM-v1 expression was included as a positive control (Hsu et al., 2019b), and a non-target control siRNA (NT siRNA; 5′-UGGUUUACAUGUCGACUAAUU-3′; Dharmacon) was included as a negative control. We measured the transcript level of ASPM-v1 using quantitative real-time PCR (qRT-PCR) using the LightCycler FastStart DNA MASTERPLUS SYBR. Green I Kit and the LightCycler System (Roche Diagnostics GmbH, Mannheim, Germany), Oligonucleotide primers were designed using Primer Bank (http://pga.mgh.harvard. edu/primerbank/index.html) and include the forward primer: GCG AAG AGT CTT AGC ACA G and the reverse primer; GTG GAA TAT CTT CTC CAA TAT CCC. We averaged the knockdown efficacy of each of the 13 siRNAs in each line and ranked their performance accordingly, thereby identifying top-ranked siRNAs that could consistently achieve a knockdown efficacy of more than 80% in all the 3 lines.

In an effort to develop the active pharmaceutical ingredient (API) of the siRNA therapeutic in our preferred embodiment of the present invention, we reason that (1) siRNAs that target different fragments on the exon 18 of the mRNA of ASPM-v1 may obviate the potential variations in the knockdown efficacy caused by chromatin conformations, and (2) a siRNA mixture consists of each siRNA at an amount half of that in a single siRNA, whereby the potential toxicity, such as off-target silencing and immune-stimulatory effects, can be theoretically mitigated. We thus selected siASPM-v1.4822 (sense strand: SEQ ID NO: 4; antisense strand: SEQ ID NO: 5), which targets Fragment I on exon 18 (ASPM-v1. e18. F1), and siASPM-v1.7636 (sense strand: SEQ ID NO: 6; antisense strand: SEQ ID NO: 7), which targets Fragment 3 on exon 18 (ASPM-v1. e18. F3). We undertook to use their 1:1 combination as the API of the ASPM-v1-targeted siRNA therapeutics for the subsequent development.

To avoid potential immunogenicity of these siRNAs and to increase their stability in serum, we added the 2′-O-methylation modification at site UA or CA in the antisense strand and at all pyrimidines in the sense strand since the 2′-O-methylation modification of siRNA formulated with an LNP has been associated with less immune activation when administered systematically (Adami, R. C., Seth, S., Harvie, P., Johns, R., Fam. R., Fosnough, K., Zhu. T., Farber, K., Mccutcheon, M., Goodman, T. T., et al. (2011). An amino acid-based amphoteric liposomal delivery system for systemic administration of siRNA. Mol Ther 19, 1141-1151). To enhance the serum stability, we additionally added two deoxy-thymidine 3′ overhangs with phosphorothioate linkage to both the sense and the antisense strand (Table 2).

TABLE 2
The chemical modifications of siASPM-v1.7636
(siASPM-v1.m7635) and siASPM-v1,4822
(siASPM-v1.m4822)

| | | Original | | Partially |
|---|---|---|---|---|
| Sequence | | sequence | Sequence | modified |
| ID | Strand | 5′-3′ | ID | sequence 5′-3′¹ |
| SiASPM- | Sense | GCCCACCAACUAU | SiASPM- | GmCGmCAmCmCAAmCm |
| v1.4822 | | UUGCAG (SEQ ID | v1.m4822 | UAmUmUmUGmCAGdT* |
| | | NO: 4) | | dT |
| | Antisense | CUGCAAAUAGUUG | | CUGmCAAAmUAGUUGG |
| | | GUGCGC (SEQ ID | | UGCGCdT*dT |
| | | NO: 5) | | |
| SiASPM- | Sense | CAGAAGACUGAUG | siASPM- | mCAGAAGAmCmUGAmU |
| v1.7636 | | GUAAAG (SEQ ID | v1.m7636 | GGmUAAAGdT*dT |
| | | NO: 6) | | |
| | Antisense | CUUUACCAUCAGU | | CUUmUACmCAUmCAGU |
| | | CUUCUG (SEQ ID | | CUUCUGAdT*dT |
| | | NO: 7) | | |
| Non-target | Sense | UGGUUUACAUGUC | mNT | mUGGmUmUmUAmCAmU |
| control | | GACUAA | SIRNA | GmUmCGAmCmUAAdT* |
| SIRNA | | | | dT |
| | Antisense | UUAGUCGACAUGU | | UmUAGUCGAmCAUGmU |
| | | AAACCA | | AAACmCAdT*dT |

m, 2′-O-methyl RNA; *, phosphorothioate linkage.
¹Underlined are modified nucleotides.

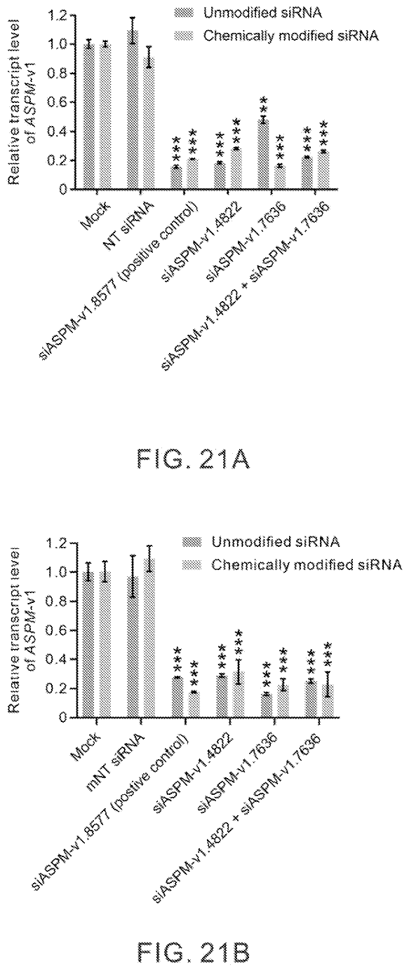

To compare and verify the knockdown efficacy of the two newly selected siRNAs and their mixture, we transduced MDA-MB-436 cells with siASPM-v1.7636, siASPM-v1.4822, their 1:1 mixture), or the non-target control siRNA (NT siRNA), with or without the chemical modifications using Lipofectamine LTX Reagent for 48 hours, after which the RNA was isolated and the transcript level of ASPM-v1 was measured using qRT-PCR. As shown in FIGS. 21A, except for the unmodified siASPM-v1.7636, all the siRNA tested could achieve a comparable knockdown efficacy of more than 70% for all the sequences tested. The chemically modified siASPM-v1.7636 and siASPM-v1.4822 could achieve a knockdown efficacy comparable to that of unmodified siRNAs except for the chemically modified siASPM-v1.7636, which resulted in a knockdown efficacy greater than that of the unmodified siASPM-v1.7636. We verified the knockdown efficacy of chemically modified siRNAs in HuH-01 cells using the same transduction method. As shown in FIG. 21B, all the sequences tested could achieve a greater than 75% knockdown efficacy, except for siASPM-v1.4822 (mean knockdown efficacy=68.6%). The collective data confirmed that the transduction with these siRNAs could achieve a satisfactory knockdown efficacy in both TNBC and HCC cells and that their performances are not affected by introducing chemical modifications.

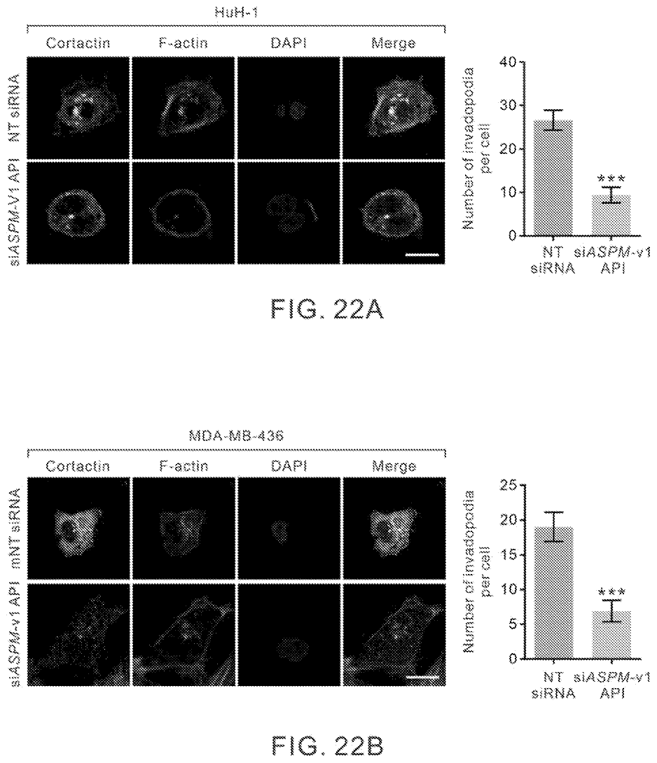

Having confirmed the gene-silencing efficacy of the 1:1 mixture of siASPM-v1.7636 and siASPM-v1.4822 (designated hereafter as siASPM-v1 API), we used it as the API of the ASPM-v1-targeted siRNA therapeutics as the preferred embodiment in the present invention. To verify the effect of siASPM-v1 API to inhibit cancer cell invadopodia formation, invasion, and development pathway activities, we transduced HuH-01 or MDA-MD-436 cells with the siASPM-v1 API or chemically modified non-target control siRNA (NT siRNA) at 100 nM using Lipofectamine LTX Reagent for 48 hours, after which the cells were plated onto the gelatin matrix (Sigma-Aldrich, G1393) as described previously (Eckert et al., 2011). The cells were seeded on gelatin for 6 hours or longer and then immunostained with anti-cortactin (4F11; Abcam, Cambridge, UK) or Alexa Fluor 647 phalloidin (staining for F-actin; Invitrogen) and evaluated the staining patterns using confocal imaging analysis using a Leica TCS SP5 confocal microscope system (Leica Microsystems GmbH, Wetzlar, Germany). The cortactin “F-actin” puncta seen under a confocal microscope represent the cross-sections of invadopodia that protrude downwardly from the cell bodies. As shown in FIGS. 22, the pre-treatment of both HhH-1 and MDA-MB-436 cells with the siASPM-v1 API could significantly reduce the number of invadopodia by averaged 64.8% (HuH-01) ˜63.7% (MDA-MB-436) compared with the cells treated with NT siRNA.

Having confirmed the inhibitory effect of siASPM-v1 API on the invadopodia formation of HCC and TNBC cells, we investigated if it can effectively inhibit the invasive capacity of cancer cells. HuH-1 cells or MDA-MB-436 cells were transduced with siASPM-v1 API at 100 nM using the Lipofectamine LTX Reagent for 48 hours, after which the cells were seeded on Transwell inserts (BD Biosciences, San Jose, CA) with a thin layer of collagen type I (BD Biosciences) in the presence of 10% FBS and allowed to invade across the collagen for 12 hours. The cells that invaded through the insert membrane were fixed, stained with SYTOX Green (Invitrogen), and counted using a fluorescence microscope. As shown in FIG. 23, in keeping with the inhibitory effect on invadopodia, the transduction of cells with siASPM-v1 API could substantially reduce the invasive capacities of both HuH-1 by averaged 89.2% and MDA-MB-436 cells (by averaged 75.3% compared with those treated with NT siRNA.

As described in our preceding findings in Example 4, ASPM-i1 contributes to the activities of multiple developmental signaling pathways, including Wnt. Hedgehog, and Notch, in cancer cells. To affirm that its functional inhibition by transducing the cells with siASPM-v1 API could suppress the activities of these development-associated signaling pathways in cancer cells, we infected HuH-1 or MDA-MB-436 cells with a lentivirus vector carrying a triple luciferase reporter of Wnt, Hedgehog (Hh), and Notch (pMuLE_EXPR_CMV-eGFP_TOP-NLuc1.1_12GLI-FLuc_CBF-GLuc; Addgene plasmid #113862) (Maier et al., 2019). In separate experiments, to further stimulate the activities of these pathways, cells were treated with recombinant human WMT3A (250 ng/ml for 16 hours; R&D Systems, Minneapolis, MN), recombinant human sonic hedgehog (SHH: 3 μg/ml for 24 hours; Sigma-Aldrich) (Lin et al., 2006), recombinant human JAG1-Fc (5 μg/ml for 24 hours; Sigma-Aldrich, Steinheim, Germany) (Dees et al., 2011) or vehicle. The Wnt, Hedgehog, and Notch reporter activity of unstimulated or stimulated cells were then measured using the Nano-Glo® Luciferase Assay System, the ONE-Glo® Luciferase Assay System, and the Renilla-Glo® Luciferase Assay System (Promega, Madison, WI), respectively.

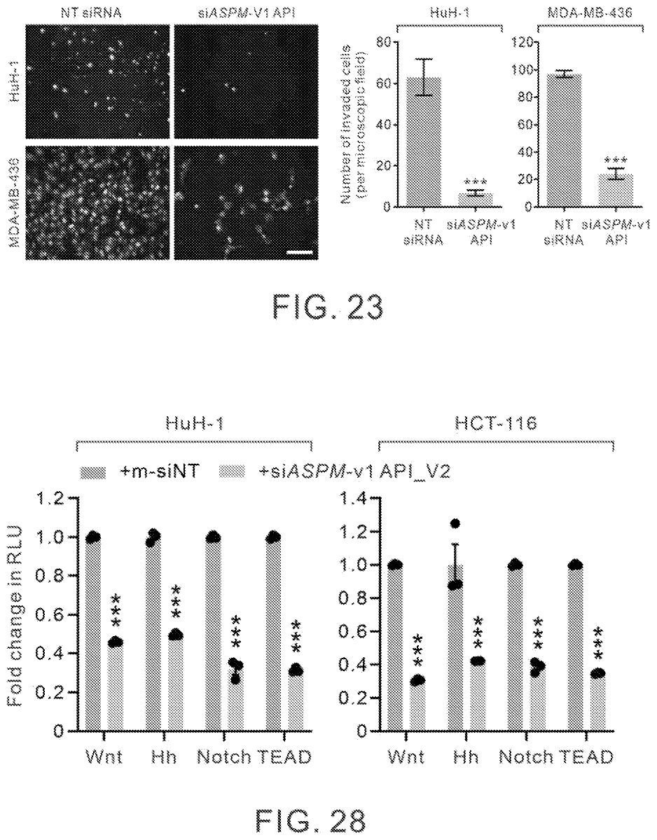

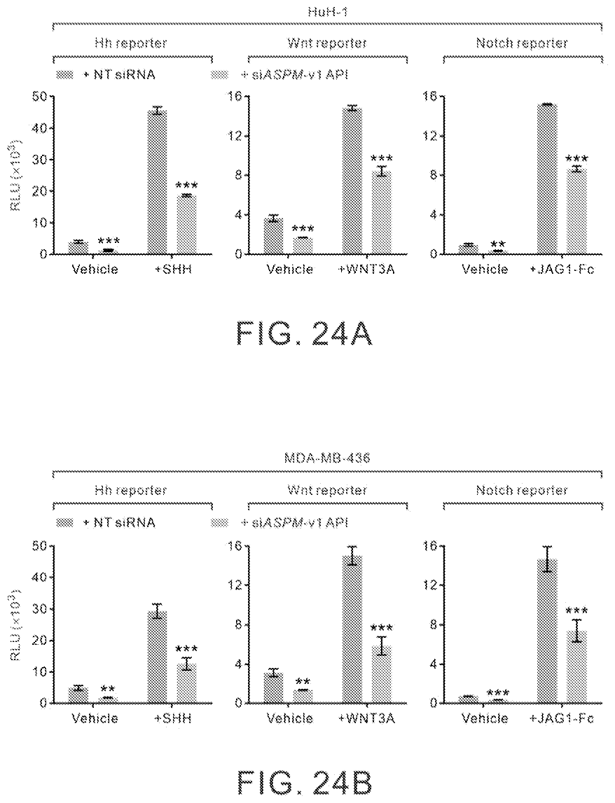

As shown in FIGS. 24, the transduction of HuH-1 cells with the siASPM-v1 API markedly reduced the Wnt, Hedgehog, and Notch reporter activities, which was even more evident in the ligand-stimulated cells. Similarly, the transduction with the siASPM-v1 API also markedly inhibited the Wnt/Hedgehog/Notch reporter activities in both unstimulated and ligand-stimulated MDA-MB-436 cells. The collective data confirmed that inhibiting the expression of ASPM-v1 by the siASPM-v1 API indeed could effectively inhibit theses oncogenic developmental pathways in different types of cancer cells.

We extended the above-mentioned siRNA selection strategy to include additional higher mammal species in the design of ASPM-v1-targeted siRNA. We selected Canis familiaris (dog) as its ASPM gene has a high percent identity with the human gene (82.69%) next to the monkey ortholog (99.52%). We aligned the exon 18 sequences of the human, canine, and monkey ASPM gene using the NCBI BLAST search, thereby identifying ten identical sub-segments in which conserved sequences were longer than 25 nucleotides. Notably, the sequences of these sub-segments were identical among human, canine, and cynomolgus monkey genes, permitting the flexible selection of relevant species in the subsequent toxicity and preclinical tests. We designed and selected candidate siRNAs using the criteria as described above.

To compare the gene-silencing effect of the candidate ASPM-v1-targeted siRNAs (siASPM-v1), we acquired the synthesized double-stranded siRNAs (Dharmacon) and transduced each into HuH-1 hepatocellular carcinoma (HCC) cells or HCT-116 colorectal cancer (CRC) cells using Lipofectamine LTX. Reagent (ThermoFisher Scientific). We measured the transcript level of ASPMv1 using qRT-PCR as described above. We ranked their performance and thereby identified two top-ranked siRNAs, including siASPM-v1.4360 (sense strand: SEQ ID NO: 8; antisense strand: SEQ ID NO: 9), and siASPM-v1.4822 (sense strand: SEQ ID NO: 4; anti-sense strand: SEQ ID NO: 5), that could consistently achieve a knockdown efficacy of more than 75% in both HuH-1 cells and HCT-116 cells. We verified that the transduction of canine A-72 fibroblasts (Bioresource Collection and Research Center (BCRC), Hsinchu, Taiwan, #60480) with the two siASPM-v1 led to a satisfactory (>75%) KD of ASPM-v1 expression. To exploit the potential utility of the selected siASPM-v1 as the active pharmaceutical ingredient (APD) of the ASPM-v1-targeted siRNA drug to extra-hepatic cancers, we tested their gene-silencing efficacy on ASPM-v1 in MDA-MB-436 triple-negative breast cancer (TNBC) cells and NCI-H209 and NCI-H146 small cell lung cancer (SCLC) cells (American Type Culture Collection, Manassas, VA). We transduced each cell line with the candidate siASPM-v1 using Lipofectamine LTX Reagent (ThermoFisher Scientific) and measured the transcript level of ASPM-v1 using qRT-PCR as described above. We verified that the two top-ranked siASPM-v1 selected from HCC and CRC cells, including siASPM-v1.4360 and siASPM-v1.4822, were the best-performed siRNAs in MDA-MB-436 TNBC cells, with the knockdown efficacy of 96.71% and 83.95%, respectively. Likewise, transduction of NCI-H209 and NCI-H146 SCLC cells with the two siASPM-v1 also achieved an excellent gene-silencing effect on ASPM-v1, achieving a knockdown effect of 84.49% and 86.31%, respectively, in NCI-H209 cells, and 81.37% and 83.77%, respectively, in NCI-H146 cells. These highly consistent and robust data support the inclusion of the two siASPM-v1 for the subsequent development of ASPM-v1-targeted siRNA therapeutics in different cancers.

siASPM-v1.4360 and siASPM-v1.4822 target different mRNA subsegments on exon 18 and we therefore designated their 1:1 combination as another version of the API of the LNP-encapsulated SiASPM-v1 (siASPM-v1 API_V2). Furthermore, to avoid the potential immunogenicity of these siRNAs and to increase their stability in serum, we added the 2′-O-methylation modification at site UA or CA in the antisense strand and at all pyrimidines in the sense strand since the 2′-O-methylation modification of siRNA formulated with an LNP has been associated with less immune activation when administered systematically (Adami, R. C., Seth, S., Harvie, P., Johns, R., Fam, R., Fosnaugh, K., Zhu, T., Farber, K., Mccutcheon, M., Goodman. T. T., et al. (2011). An amino acid-based amphoteric liposomal delivery system for systemic administration of siRNA. Mol Ther 19, 1141-1151). To enhance the serum stability, we added two deoxy-thymidine 3′ overhangs with phosphorothioate linkage to the sense and the antisense strand. The chemically modified siRNAs were named “siANPM-v1.m4360-4378” and “siASPM-v1.m4822-4840”, respectively, wherein “m” indicates “modified” (Table 3).

TABLE 3
The chemical modifications of
siASPM-v1.4360 (siASPM-v1.m4360)

| | | | | Partially |
|---|---|---|---|---|
| | | Original | | modified |
| Sequence | | sequence | Sequence | sequence |
| ID | Strand | 5′-3′ | ID | 5′-3′¹ |
| SLASPM-V1. | Sense | CCUGCAAU | SiASPM- | mCmCmUGm |
| 4360 | | CUAGGAUA | v1.m4360 | CAAmUmCm |
| | | AGA | | UAGGAmUAA |
| | | (SEQ ID | | GAdT*dT |
| | | NO: 8) | | |
| | Antisense | GGACGUUA | | GGACGUmU |
| | | GAUCCUA | | AGAUCCm |
| | | UUCU | | UAUUCUd |
| | | (SEQ ID | | T*dT |
| | | NO: 9) | | |

m, 2′-O-methyl RNA; *, phosphorothioate linkage.
¹Underlined are modified nucleotides.

To compare and verify the gene-silencing efficacy, we transduced Huh-1 and HCT-116 cells with siASPM-v1.m4360, siASPM-v1.m4822, their 1:1 mixture (siASPM-v1 API_V2), or non-target SÍRNA (siNT), with or without the chemical modifications as shown in Table 2 and Table 3 using Lipofectamine LTX Reagent for 48 hours, after which the RNA was isolated, and the transcript level of ASPM-v1 was measured using qRT-PCR. As shown in

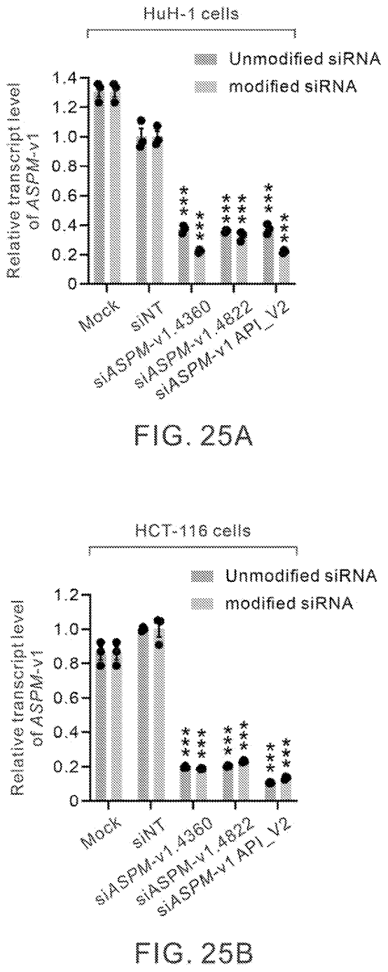

FIG. 25A, all the siRNA tested could achieve a gene-silencing efficacy on ASPM-v1 for approximately 70% in HuH-1 HCC cells. Of note, the chemically modified siRNAs could achieve a knockdown efficacy (approximately 75%) slightly better than of unmodified siRNAs. Likewise, in HCT-116 CRC cells (

FIG. 25B), all the sequences tested achieved an averaged knockdown efficacy of 82.26%. These data demonstrated that siASPM-v1.m4360-4378, siASPM-v1.m4822-4840, or their 1:1 mixture (i.e., siASPM-v1 API_V2) could all achieve a satisfactory gene-silencing effect on ASPM-v1 in both HCC and CRC cells and that their performances were unaffected by introducing chemical modifications.

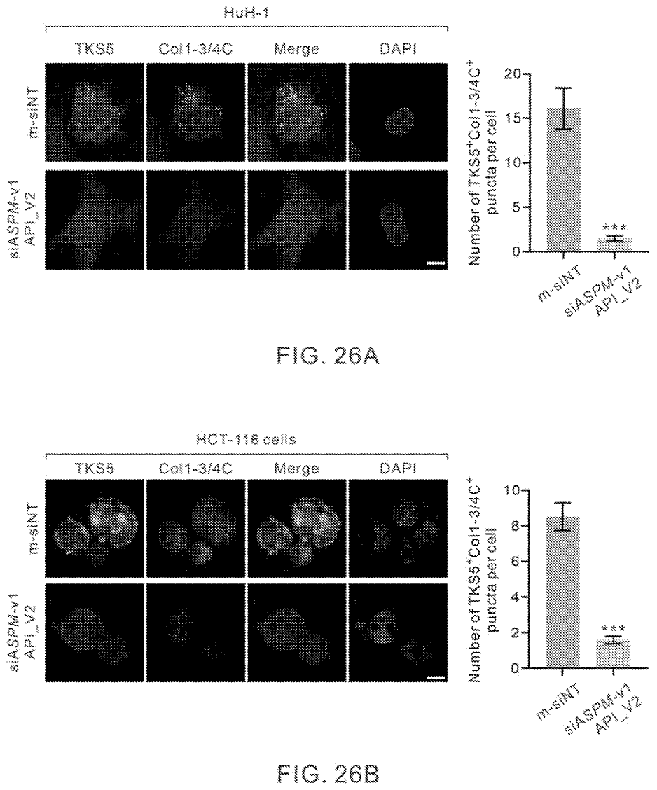

Next, we pursued functional characterization of siASPM-v1 API_V2 in terms of its efficacy in inhibiting cancer cell invadopodia formation, invasion, and development-associated pathway activities. First, we transduced HuH-1 or HCT-116 cells with siASPM-v1 API_V2 or the chemically modified non-target siRNA (m-siNT) at 100 nM using Lipofectamine LTX Reagent for 48 hours, after which the cells were plated onto the gelatin matrix (Sigma-Aldrich, G1393). The cells were seeded on gelatin for 12 hours and then immunostained with the invadopodia marker TKS5 (anti-TKS5; 1:200; clone 13H6.3. Merck, Burlington MA) or anti-Col1-3/4C (collagen type I cleavage site; 1:25, ImmunoGlobe Antikörpertechnik GmbH, Himmelstadt, Germany), and evaluated the staining patterns using confocal imaging analysis using a Leica TCS SP5 confocal microscope system (Leica Microsystems GmbH, Wetzlar, Germany). The TKS5⁺Col1-3/4C⁺ puncta seen under a confocal microscope represent the cross-sections of functional invadopodia that protrude downwardly from the cell bodies and degrade the surrounding collagen 1 matrices. As shown in FIG. 26, the pre-treatment of both HhH-1 and HCT-116 cells with siASPM-v1 API_V2 could significantly reduce the number of invadopodia by an averaged 90.69% in HuH-1 cells or 81.31% in HCT-116 cells compared with the cells treated with siNT.

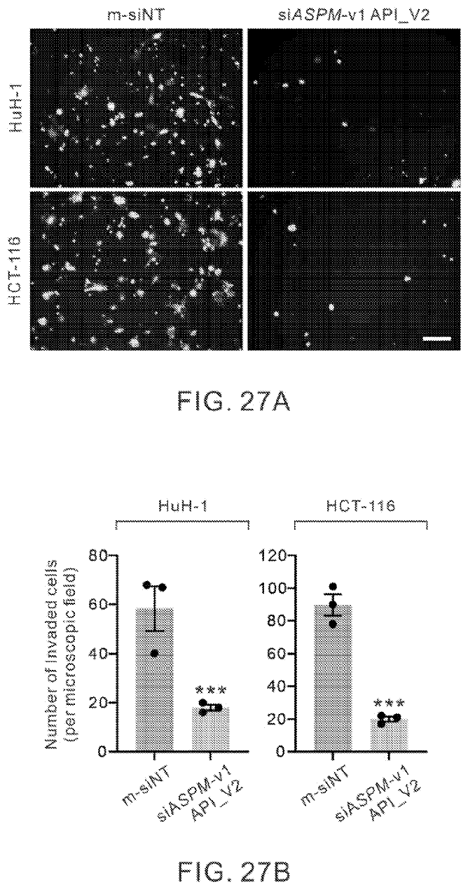

Having confirmed the inhibitory effect of siASPM-v1 API_V2 on the invadopodia formation of cancer cells, we next investigated if it can effectively inhibit the invasive capacity of cancer cells. To this end, HuH-1 or HCT-116 cells were transduced with siASPM-v1 API_V2 at 100 nM using Lipofectamine LTX Reagent for 48 hours, after which the cells were seeded on Transwell inserts (BD Biosciences, San Jose, CA) with a thin layer of collagen type I (BD Biosciences) in the presence of 10% FBS and allowed to invade across the collagen for 12 hours. The cells that invaded through the insert membrane were fixed, stained with SYTOX Green (Invitrogen), and counted using a fluorescence microscope. As shown in FIG. 27, in keeping with the inhibitory effect on invadopodia, the transduction of cells with siASPM-v1 API_V2 could substantially reduce the invasive capacities of HuH-1 by an averaged 69.1% and HCT-116 cells by an averaged 77.7% compared with those treated with m-siNT.

ASPM-i1 has been shown to critically control to the activities of multiple development-associated and oncogenic signaling pathways, including the Wnt. Hedgehog (Hh), and Notch pathways. We recently uncovered that ASPM-i1 also increased the stability of Yes-associated protein (YAP) and PDZ-binding motif (TAZ), both being the co-activators of the TEAD transcriptional factor, in cancer cells. To affirm that its functional inhibition by transducing the cells with siASPM-v1 API_V2 could suppress the activities of the aforementioned these development- and stemness-associated pathways in cancer cells, we infected HuH-1 or HCT-116 cells with a lentivirus vector carrying a triple luciferase reporter of Wat, Hh, and Notch (pMuLE_EXPR_CMV-eGFP_TOP-NLuc1.1_12GLI-FLuc_CBF-OLuc; Addgene plasnud #113862) (Maier et al., 2019) or the Hippo pathway transcriptional factor TEAD (BPS Bioscience #79833. San Diego, CA). Cells were treated with recombinant human WNT3A (250 ng/ml for 16 hours; R&D Systems, Minneapolis, MN), recombinant human sonic hedgehog (SHH; 3 μg/ml for 24 hours; Sigma-Aldrich), recombinant human JAG1-Fc (5 μg/ml for 24 hours; Sigma-Aldrich, Steinheim, Germany), respectively, to stimulate the activities of the War, Hh, or Notch pathway. The Wnt, Hh, Notch, and TEAD reporter activity of unstimulated or stimulated cells were then measured using the Nano-Glo® Luciferase Assay System (Wat repoter), the ONE-Glo® Luciferase Assay System (Hh and TEAD reporter), and the Renilla-Glo® Luciferase Assay System (Notch reporter) (Promiega, Madison, WI). As shown in FIG. 28, the transduction of HuH-1 cells with siASPM-v1 API_V2 markedly reduced the reporter activities of all the pathways tested, including the Wnt, Hh, Notch, and TEAD, affirming that inhibiting the expression of ASPM-v1 by siASPM-v1 APL_V2 could effectively inhibit multiple oncogenic and development-associated pathways in cancer cells

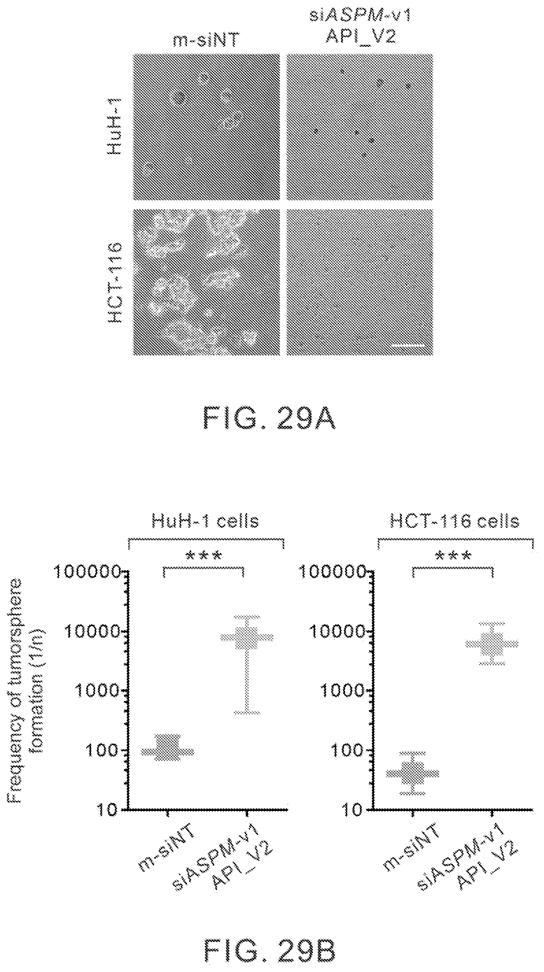

The ability to form large three-dimensional sphere-like structures, or “tumorspheres”, under serum-free and anchorage-independent conditions reflects the tumorigenic potential of cancer cells. Given that the knockdown of ASPM-v1 expression prominently inhibited the development- and stemness-associated Wnt, Hh, and Notch pathways, and the transcriptional activity of YAP/TEAD, we investigated if treatment of cancer cells with siASPM-v1 API_V2 could inhibit their tumorsphere-forming capacity. HaH-1 hepatocellular carcinoma cells or HCT-116 colorectal cancer cells were transduced with siASPM-v1 API_V2 or m-siNT at 100 nM using Lipofectamine LTX Reagent for 48 hours, after which the cells were plated in limiting dilution (10000, 1000, 100, and 10 cells per well) in 24-well nonadherent culture plates. The presence of tumorspheres were evaluated after ten days after ten days. The data from the limiting dilution assay were analyzed and plotted using the ELDA software (http://bioinf.wehi. edu.au/software/elda/index.html). As shown in FIG. 29, the transduction of HhH-1 or HCT-116 cells with siASPM-v1 API_V2 markedly inhibited the tumorsphere-forming capacity of HuH-1 or HCT-116 cells, underscoring its therapeutic potential in hepatocellular carcinoma and colorectal cancer.

1. A polynucleotide, comprising a nucleotide sequence complimentary to the mRNA of the ASPM gene with the nucleotide sequence shown in SEQ ID NO: 1: or a nucleotide sequence comprising a contiguous segment having at least 70%, at least 80%, or at least 90% sequence identity to the nucleotide sequence complimentary to SEQ ID NO: 1.

2. The polynucleotide of claim 1, which comprises a nucleotide sequence complementary to the mRNA encoded by exon 18 of the transcript variant 1 of the human ASPM gene with the nucleotide sequence shown in SEQ ID NO:3 or a nucleotide sequence comprising a contiguous segment having at least 70%, at least 80%, or at least 90% sequence identity to the nucleotide sequence complimentary to SEQ ID NO:3.

3. The polynucleotide of claim 1, wherein the polynucleotide is shRNA, siRNA or dsRNA.

4. The polynucleotide of claim 1, wherein the polynucleotide is an siRNA molecule, wherein said siRNA molecule comprises: (a) a duplex region; and (b) either no overhang regions or at least one overhang region, wherein each overhang region contains six or fewer nucleotides, wherein the duplex region consists of a sense region and an antisense region, wherein said sense region and said antisense region together form said duplex region, and said antisense region and said sense region are each 15-30 nucleotides in length and said antisense region comprises a sequence that is the complement of a sequence selected from SEQ ID NO: 3.

5. The polynucleotide of claim 4, wherein the antisense region and the sense region are each 15-25 bases in length.

6. The polynucleotide of claim 4, wherein the siRNA molecule is a chemically synthesized double stranded siRNA molecule, wherein: (a) each strand of said double stranded siRNA molecule is between 15 and 30 nucleotides in length; and (b) one strand of said siRNA molecule comprises a sequence that is the complement of a sequence selected from SEQ ID NO: 3, 5, 7 or 9.

7. The polynucleotide of claim 4, wherein the siRNA molecule comprises a sense strand and an antisense strand, wherein said sense strand comprises at least 15 contiguous nucleotides differing by no more than 3 nucleotides from SEQ ID NO: 4, and said antisense strand comprises at least 15 contiguous nucleotides differing by no more than 3 nucleotides from SEQ ID NO: 5.

8. The polynucleotide of claim 7, wherein the siRNA molecule comprises a sense strand and an antisense strand, wherein said sense strand comprises the nucleotide sequence as set forth in SEQ ID NO:4 and said antisense strand comprises the nucleotide sequence as set forth in SEQ ID NO: 5.

9. The polynucleotide of claim 4, wherein the siRNA molecule comprises a sense strand and an antisense strand forming another double-stranded RNA dimer, wherein said sense strand comprises at least 15 contiguous nucleotides differing by no more than 3 nucleotides from SEQ ID NO: 6, and said antisense strand comprises at least 15 contiguous nucleotides differing by no more than 3 nucleotides from SEQ ID NO: 7.

10. The polynucleotide of claim 2, wherein the siRNA molecule comprises a sense strand and an antisense strand, wherein said sense strand comprises the nucleotide sequence as set forth in SEQ ID NO:6 and said antisense strand comprises the nucleotide sequence as set forth in SEQ ID NO: 7.

11. The polynucleotide of claim 4, wherein the siRNA molecule comprises a sense strand and an antisense strand forming another double-stranded RNA dimer, wherein said sense strand comprises at least 15 contiguous nucleotides differing by no more than 3 nucleotides from SEQ ID NO: 8, and said antisense strand comprises at least 15 contiguous nucleotides differing by no more than 3 nucleotides from SEQ ID NO: 9

12. The polynucleotide of claim 11, wherein the siRNA molecule comprises a sense strand and an antisense strand, wherein said sense strand comprises the nucleotide sequence as set forth in SEQ ID NO:8 and said antisense strand comprises the nucleotide sequence as set forth in SEQ ID NO: 9.

13. (canceled)

14. The polynucleotide of claim 4, wherein one or both of the sense strand and the antisense strand are further modified as modified siRNA.

15. The polynucleotide of claim 14, wherein the modified siRNA is modified with one or more of the following: a 2′-O-methyl modified nucleotide, a 2′-fluorophoramidate, a 3′-terminal deoxy-thymine nucleotide, a non-natural base comprising a nucleotide, a nucleotide comprising a 5′phosphorothioate group, and a terminal nucleotide linked to a cholesteryl derivative and a dodecanoic acid bisdecylamide group.

16. The polynucleotide of claim 14, wherein the modified siRNA comprises 10% to about 30% of the nucleotides in the double stranded region comprise 2′-O-methyl (2′OMe) nucleotides, comprises 2′OMe nucleotides on both strands of the modified siRNA.

17. (canceled)

18. (canceled)

19. (canceled)

20. A pharmaceutical composition comprising a polynucleotide of claim 1 and a pharmaceutical carrier, diluent and/or adjuvant.

21. A nanoparticle comprising the polynucleotide of claim 1 and lipid nanoparticle (LNPs), liposome, micelle, virosome, nucleic acid complex, and any mixture thereof.

22. A method for inhibiting growth, loco-regional spreading, and distant metastasis of a malignant tumor and/or treating a solid tumor and/or tumor metastasis in a subject, comprising administrating the polynucleotide of claim 1 to the subject.

23. The method of claim 22, wherein the solid cancer is selected from hepatocellular carcinoma, colorectal cancer, breast cancer, pancreatic cancer, gastric cancer, and lung cancer.


SEQUENCE LISTING (9 sequences)

SEQ ID NO: 1 - RNA, 10906 nt, Homo sapiens
  source 1..10906: mol_type = mRNA
attggtggaggcggcaagtttaaacagagtcaaaacgccatacttgtttggctcctctttttaatttgcgagtttattgggcttgttttctgttttctagggagtaggttagtggaaaagaaaaagggccgaattcactcccacgacctctacagccgcccctgaggggaagcggtcagcgtaagtcccggatccccgctccggagccgcctcgtgggagcggggcaaggagatccaggaggggtctcgaatctgccatggcgaaccggcgagtggggcgaggctgctgggaagtgagcccgaccgagcggaggccgcccgcggggctgcggggccccgcggccgaggaggaggcgtcttccccgccggtcctgtctctcagccacttctgcaggtctcctttcctttgcttcggggacgttctcctgggagcctcacggacgctgtctctggccctagacaaccctaacgaggaggtggcagaagtgaagatctcccacttcccggccgcggacctgggcttcagtgtgtcgcagcgctgtttcgtgttgcagcctaaagagaaaattgttatttctgttaactggacaccactcaaagaaggccgagtaagagagattatgacatttcttgtaaatgatgttctgaaacaccaagctatattactaggaaatgcagaagagcagaaaaagaaaaagaggagtctttgggataccattaaaaagaagaaaatttcagcctctacaagtcacaacagaagggtttcaaatattcagaatgttaataaaacatttagtgtttcccaaaaagttgacagagttaggagcccactacaagcttgtgaaaacttggctatgaatgaaggcggtcccccaacagaaaacaattctttaatacttgaagaaaataaaatacccatatcacctattagccctgctttcaatgaatgccatggtgcaacttgcttgccactctctgtacgtcgatctactacctactcatctcttcatgcatcagaaaatagggaactattaaatgtacacagtgccaacgtttcaaaagtttcttttaatgagaaagctgtaactgaaacttcctttaattccgtaaatgttaatggccaaagaggagagaatagtaaacttagtcttacccccaactgttcttcaactttgaacattacacaaagccaaatacattttctaagtccagattcttttgtaaataatagtcatggagctaataatgaactagaattagtaacatgtctttcatcagatatgtttatgaaagataattcacagcctgtgcatttggaatcaacaattgcacatgaaatttatcagaaaattttaagtccagattctttcataaaagataattatggactaaatcaggatctagaatcagagtcagttaatcctattttatcccctaatcaatttttaaaagataacatggcatatatgtgtacatctcagcaaacatgtaaagtaccattatcaaatgaaaattctcaagtcccacagtctcctgaagattggagaaaaagtgaagtttcgccacgtattcctgaatgtcagggttcaaaatctcccaaagctatttttgaagaactagtagaaatgaagtcaaattactacagttttataaaacaaaataatcctaaattttctgcagttcaggatatttctagtcatagccacaataaacaacctaagagacgtccaatactttctgccactgttactaaaaggaaggccacctgtaccagagaaaaccaaactgagattaataaaccaaaagcaaaaagatgtctcaacagtgcagtgggtgaacatgaaaaagtaataaataatcaaaaggaaaaagaagattttcattcttatcttccaattatagatccaatattaagtaaatctaagagttataaaaacgaggtaacaccctcttcgacaacagcttcagttgctcggaaaagaaagagcgatggaagcatggaagatgcaaatgtgagagttgcaattacagaacatacagaagtgcgagaaatcaaaagaatccatttttctccctcagagcctaaaacatcagctgttaagaaaacaaaaaatgtgacaacacccatctcaaaacgtattagcaacagagagaaattaaacctgaagaagaaaactgatttatcaatattcagaactccaatttctaaaacaaacaaaaggacaaaacccattatcgctgtggcacagtccagtttgaccttcataaaaccattaaaaacagatattcccagacacccgatgccatttgctgcaaaaaacatgttttatgatgaacgctggaaggaaaagcaggaacagggcttcacttggtggttaaattttatattaacccctgatgacttcactgtaaaaacaaatatttctgaagtaaatgctgctactcttcttttgggaatagagaatcaacataaaataagtgttcctagagcacctacaaaagaggaaatgtctctcagagcttatactgctcggtgtaggttaaacagactacgtcgtgcagcatgccgtttgtttacttctgaaaaaatggttaaagctattaaaaagcttgaaattgaaattgaagctaggcggttaattgttcgaaaagatagacacctatggaaagatgtgggagaacgtcagaaagtcctgaattggctgttgtcctacaatcctttgtggcttcgaattggtctagagacaacttatggagaactcatatctttggaagataacagtgatgtcacagggttggctatgtttattctgaatcgcctactttggaatcctgatatagcagctgagtatagacaccccactgttcctcacctgtatagagatggtcatgaagaagctttgtccaagtttacattgaaaaagttattgttgttggtctgttttcttgattatgctaaaatttccagactcattgatcatgatccttgtctcttctgtaaagatgccgaattcaaggctagtaaagaaatccttttggctttttcacgagatttcctaagtggtgaaggtgacctttcccgtcaccttggcttattgggattacctgttaaccatgttcagacaccatttgatgaatttgattttgccgttacaaatcttgccgtagacttgcaatgtggagtgcgccttgtgcgaaccatggaacttctcacacagaactgggacctctcaaagaaactcaggattccggcaataagtcgtcttcaaaagatgcacaatgttgacattgttcttcaagttcttaaatcacgaggaattgaattaagtgatgagcatggaaatacaattctatctaaggatattgtggataggcacagagaaaaaactctcaggttgctttggaaaatagcgtttgcttttcaggtggatatttcccttaacttagatcaattaaaggaagaaattgcctttctaaaacacacaaagagtataaagaaaacaatatctctactatcatgccattctgatgatcttattaataagaaaaaaggcaaaagggatagtggttcctttgaacaatatagtgaaaacataaagttattgatggattgggtaaatgctgtttgtgccttctataataaaaaggtggagaattttacagtgtctttctcagacggccgtgtgttatgttacctgatccaccattaccatccttgctatgtgccatttgacgctatatgtcagcgtactactcaaactgtggaatgtacgcaaactggttcagtggtattaaattcatcatctgaatctgatgacagttctctggatatgtctcttaaagcatttgatcatgaaaatacttcagagctatacaaagagctcctagaaaatgaaaagaaaaattttcacttggttaggtctgcagttagagaccttggtggaatacctgctatgattaatcattcagatatgtcaaatacaattccagatgaaaaggtggttattacctatttgtcatttctttgtgcaaggcttttggatcttcgtaaagaaataagagctgctcgactcatacaaacaacatggagaaaatataaactaaaaacagatctcaaacgccatcaggagagagagaaagctgcaagaattattcaattggctgtaatcaattttctagcaaaacaaagattgagaaaaagagttaatgcagcactcgtcattcagaaatattggcgaagagtcttagcacagagaaaattattaatgttaaaaaaggaaaagctggaaaaagttcaaaataaagcagcatcacttattcagggatattggagaagatattccactagacaaagatttctgaaattgaaatattattcaatcatcctgcaatctaggataagaatgataattgctgttacatcttataaacgatatctttgggctacagttacaattcagaggcattggcgtgcttatttaagaagaaaacaagatcaacaaagatatgaaatgctaaaatcatcaactcttataatccaatctatgttcagaaaatggaagcaacgtaaaatgcaatcacaagtaaaagctacagtaatattgcaaagagcttttagagaatggcatttaagaaaacaagctaaagaagaaaattctgctattatcatacaatcatggtatagaatgcataaagaattacggaaatatatttatattagatcttgtgttgttatcattcagaaaagatttcggtgctttcaagcccaaaagttatataaaagaagaaaagagtccatactaaccatccagaagtactacaaagcatatctgaaaggaaagattgagcgcaccaactatttgcagaaacgagctgcagccattcaattacaagctgcttttaggagactgaaagctcataatttatgtagacaaattagagctgcttgtgttattcagtcatactggagaatgagacaagacagagttcgatttttaaaccttaagaagactattatcaaatttcaggcacatgtaagaaaacatcaacaacgacagaaatataagaagatgaagaaagcagctgttataattcagactcatttccgagcttatatttttgccatgaaagttctagcatcttaccagaaaacacgctctgctgtcattgtgctgcagtctgcatatagagggatgcaagccaggaaaatgtatattcacatcctcacatctgttataaagattcaatcatattatcgtgcttatgtttctaaaaaggaatttttgagcctaaaaaatgctacaataaaattgcagtcaactgttaagatgaaacaaacacgtaaacaatatttgcatttaagagcagctgcactatttatccagcaatgttaccgttccaaaaaaatagctgcacaaaagagagaagagtatatgcagatgcgggaatcttgtatcaaactgcaagcatttgttagaggataccttgtccgaaagcagatgaggttacaaagaaaagctgttatttcactacagtcttatttcagaatgagaaaggctcggcagtattatctgaaaatgtataaagcaattattgtcattcagaattactatcatgcatacaaagcacaggtcaatcagaggaagaacttcttgcaagtcaaaaaagcagctacttgcttgcaagcagcttacagaggttataaagtacgccagctaatcaaacaacaatctatagctgctcttaaaattcagtctgcttttagaggctataataaaagggtaaaatatcaatctgtgcttcaatctataataaagattcagagatggtacagggcgtacaagactcttcatgatacaagaacacattttttgaagacaaaggcagctgtgatttccctccagtctgcttatcgtggctggaaggttcggaaacagattagaagggaacatcaagctgccttgaagattcagtctgcttttagaatggccaaggcccagaaacagtttagattgtttaaaacagcagcattagtcatccagcaaaatttcagagcatggactgcaggaaggaagcaatgtatggagtatattgaactccgtcatgcggtactggtgcttcaatctatgtggaagggaaaaacactgagaagacagcttcaaaggcaacataaatgtgctatcatcatacagtcatactatagaatgcatgtgcaacaaaagaagtggaaaatcatgaaaaaagctgctcttctgattcaaaagtattatagggcttacagtattggaagagaacagaatcatttatatttgaaaacaaaagcagctgtagtaactttacagtcagcttatcgtggtatgaaagtgagaaaaagaataaaggattgcaacaaagcagcagtcactatacagtctaaatacagagcttacaaaaccaaaaagaaatatgcaacctatagagcttcagctattataattcagagatggtatcgaggtattaaaattacaaaccatcagcataaggagtatcttaatttgaagaagacagcaattaaaatccaatctgtttatagaggtattagagttagaagacatattcaacacatgcacagggcagccacttttattaaagccatgtttaaaatgcatcagtcaagaataagttaccatacaatgagaaaagcagctattgttattcaagtaagatgtagagcatattatcaaggtaaaatgcagcgtgaaaagtacctgacaattttgaaagctgttaaagtccttcaggcaagttttagaggagtaagagttagacggactcttagaaagatgcagactgcagcaacactcattcagtcaaactacagaagatacagacagcaaacatactttaataagttaaagaaaataacaaaaacagtacagcaaagatactgggcaatgaaagaaagaaacatacaatttcaaaggtataacaaactgaggcattctgtaatatacattcaggctatttttaggggaaagaaagctagaagacatttaaaaatgatgcatatagccgcaactctcattcagaggagatttagaactctaatgatgagaagaagattcctctctctcaagaaaactgctattttgattcagagaaaatatcgggcacatctttgtacaaagcatcacttacagttccttcaggtacaaaatgcagttattaaaatccagtcatcatacagaagatggatgataaggaaaaggatgcgagagatgcacagggctgctactttcatccagtctactttcagaatgcacagattacatatgagatatcaggctttgaaacaggcctccgttgtgatccaacagcaataccaagcaaatagagctgcaaaactgcagaggcagcattatctcagacaaagacactctgctgtgatccttcaggctgcattcaggggtatgaaaactagaagacatttgaagagtatgcattcctctgcaacccttattcagagtaggtttagatcattactggtgaggagaagattcatttccctcaaaaaagctactatttttgttcagaggaaatatcgagccaccatttgtgccaaacataaattgtaccaattcttgcacttaagaaaggcagccattacaatacagtcatcttacagaagactgatggtaaagaagaagttacaagaaatgcaaagggctgcagttctcattcaggctactttcaggatgtacagaacatatattacatttcagacttggaaacatgcttcaattctaattcagcaacattatcgaacatatagagctgcaaaattacaaagagaaaattatatcagacaatggcattctgctgtggttattcaggctgcatataaaggaatgaaagcaagacaacttttaagggaaaaacacaaagcttctatcgtaatacaaagcacctacagaatgtataggcagtattgtttctaccaaaagcttcagtgggctacaaaaatcatacaagaaaaatatagagcaaataaaaagaaacagaaagtatttcaacacaatgaacttaagaaagagacttgtgttcaggcaggttttcaggacatgaacataaaaaaacagattcaggaacagcaccaggctgccattattattcagaagcattgtaaagcctttaaaataaggaagcattatctccaccttagagcaacagtagtttctattcaaagaagatacagaaaactaactgcagtgcgtacccaagcagttatttgtatacagtcttattacagaggctttaaagtacgaaaggatattcaaaatatgcaccgggctgccacactaattcagtcattctatcgaatgcacagggccaaagttgattatgaaacaaagaaaactgcaattgtggttatacagaattattataggttgtatgttagagtaaaaacagaaagaaaaaactttttagcagttcagaaatctgtacgaactattcaggctgcttttagaggcatgaaagttagacaaaaattgaaaaatgtatcagaggaaaagatggcagccattgttaaccaatctgcactctgctgttacagaagtaaaactcagtatgaagctgttcaaagtgaaggtgttatgattcaagagtggtataaagcttctggccttgcttgttcacaggaagcagagtatcattctcaaagtagggctgcagtaacaattcaaaaagctttttgtagaatggtcacaagaaaactggaaacacagaaatgtgctgccctacggattcagttcttccttcagatggctgtgtatcggagaagatttgttcagcagaaaagagctgctatcactttacagcattattttaggacgtggcaaaccagaaaacagtttttactatatagaaaagcagcagtggttttacaaaatcactacagagcatttctgtctgcaaaacatcaaagacaagtctatttacagatcagaagcagtgttatcattattcaagctagaagtaaaggatttatacagaaacggaagtttcaggaaattaaaaatagcaccataaaaattcaggctatgtggaggagatatagagccaagaaatatttatgtaaagtgaaagctgcctgcaagattcaagcctggtatagatgttggagagcacacaaagaatatctagctatattaaaagctgttaaaattattcaaggttgcttctataccaaactagagagaacacggtttttgaatgtgagagcatcagcaattatcattcagagaaaatggagagctatacttcctgcaaagatagctcatgaacacttcttaatgataaaaagacatcgagctgcttgtttgatccaagcacattatagaggatataaaggaaggcaggtctttcttcggcagaaatctgctgctttgatcatacaaaaatatatacgagccagggaggctggaaagcatgaaaggataaaatatattgaatttaaaaaatctacagttatcctacaagcactggtgcgtggttggctagtacgaaaaagatttttagaacagagagccaaaattcgacttcttcacttcactgcagctgcatattatcacctgaatgctgttagaattcaaagagcctataaactttacctggctgtgaagaatgctaacaagcaggttaattcagtcatctgtattcagagatggtttcgagcaagattacaagaaaagagatttattcagaaatatcatagcatcaaaaagattgagcatgaaggtcaagaatgtctgagccagcgaaatagggctgcatcagtaatacagaaagcagtgcgccattttctcctccgtaaaaagcaggaaaaattcactagtggaatcattaaaattcaggcattatggagaggctattcttggaggaagaaaaatgattgtacaaaaattaaagctatacgactaagtcttcaagttgttaatagggagattcgagaagaaaacaaactctacaaaagaactgcacttgcacttcattaccttttgacatataagcacctttctgccattcttgaggccttaaaacacctagaggtagttactagattgtctccactttgttgtgagaacatggcccagagtggagcaatttctaaaatatttgttttgatccgaagttgtaatcgcagtattccttgtatggaagtcatcagatatgctgtgcaagtcttgcttaatgtatctaagtatgagaaaactacttcagcagtttatgatgtagaaaattgtatagatatactattggagcttttgcagatataccgagaaaagcctggtaataaagttgcagacaaaggcggaagcatttttacaaaaacttgttgtttgttggctattttactgaagacaacaaatagagcctctgatgtacgaagtaggtccaaagttgttgaccgtatttacagtctctacaaacttacagctcataaacataaaatgaatactgaaagaatactttacaagcaaaagaagaattcttctataagcattccttttatcccagaaacacctgtaaggaccagaatagtttcaagacttaagccagattgggttttgagaagagataacatggaagaaatcacaaatcccctgcaagctattcaaatggtgatggatacgcttggcattccttattagtaaatgtaaacattttcagtatgtatagtgtaaagaaatattaaagccaatcatgagtacgtaaagtgatttttgctctccgtgtacaacttttaaaatctgactttgttttaaaaaaacataaactgttcattacattcttcatttttatcatttatagttttatgcatgtaataaactaatatgtcataagatgaaaaaaaaaaaaaaaaaaa

SEQ ID NO: 2 - protein, 3477 aa, Homo sapiens
MANRRVGRGCWEVSPTERRPPAGLRGPAAEEEASSPPVLSLSHFCRSPFLCFGDVLLGASRTLSLALDNPNEEVAEVKISHFPAADLGFSVSQRCFVLQPKEKIVISVNWTPLKEGRVREIMTFLVNDVLKHQAILLGNAEEQKKKKRSLWDTIKKKKISASTSHNRRVSNIQNVNKTFSVSQKVDRVRSPLQACENLAMNEGGPPTENNSLILEENKIPISPISPAFNECHGATCLPLSVRRSTTYSSLHASENRELLNVHSANVSKVSFNEKAVTETSFNSVNVNGQRGENSKLSLTPNCSSTLNITQSQIHFLSPDSFVNNSHGANNELELVTCLSSDMFMKDNSQPVHLESTIAHEIYQKILSPDSFIKDNYGLNQDLESESVNPILSPNQFLKDNMAYMCTSQQTCKVPLSNENSQVPQSPEDWRKSEVSPRIPECQGSKSPKAIFEELVEMKSNYYSFIKQNNPKFSAVQDISSHSHNKQPKRRPILSATVTKRKATCTRENQTEINKPKAKRCLNSAVGEHEKVINNQKEKEDFHSYLPIIDPILSKSKSYKNEVTPSSTTASVARKRKSDGSMEDANVRVAITEHTEVREIKRIHFSPSEPKTSAVKKTKNVTTPISKRISNREKLNLKKKTDLSIFRTPISKTNKRTKPIIAVAQSSLTFIKPLKTDIPRHPMPFAAKNMFYDERWKEKQEQGFTWWLNFILTPDDFTVKTNISEVNAATLLLGIENQHKISVPRAPTKEEMSLRAYTARCRLNRLRRAACRLFTSEKMVKAIKKLEIEIEARRLIVRKDRHLWKDVGERQKVLNWLLSYNPLWLRIGLETTYGELISLEDNSDVTGLAMFILNRLLWNPDIAAEYRHPTVPHLYRDGHEEALSKFTLKKLLLLVCFLDYAKISRLIDHDPCLFCKDAEFKASKEILLAFSRDFLSGEGDLSRHLGLLGLPVNHVQTPFDEFDFAVTNLAVDLQCGVRLVRTMELLTQNWDLSKKLRIPAISRLQKMHNVDIVLQVLKSRGIELSDEHGNTILSKDIVDRHREKTLRLLWKIAFAFQVDISLNLDQLKEEIAFLKHTKSIKKTISLLSCHSDDLINKKKGKRDSGSFEQYSENIKLLMDWVNAVCAFYNKKVENFTVSFSDGRVLCYLIHHYHPCYVPFDAICQRTTQTVECTQTGSVVLNSSSESDDSSLDMSLKAFDHENTSELYKELLENEKKNFHLVRSAVRDLGGIPAMINHSDMSNTIPDEKVVITYLSFLCARLLDLRKEIRAARLIQTTWRKYKLKTDLKRHQEREKAARIIQLAVINFLAKQRLRKRVNAALVIQKYWRRVLAQRKLLMLKKEKLEKVQNKAASLIQGYWRRYSTRQRFLKLKYYSIILQSRIRMIIAVTSYKRYLWATVTIQRHWRAYLRRKQDQQRYEMLKSSTLIIQSMFRKWKQRKMQSQVKATVILQRAFREWHLRKQAKEENSAIIIQSWYRMHKELRKYIYIRSCVVIIQKRFRCFQAQKLYKRRKESILTIQKYYKAYLKGKIERTNYLQKRAAAIQLQAAFRRLKAHNLCRQIRAACVIQSYWRMRQDRVRFLNLKKTIIKFQAHVRKHQQRQKYKKMKKAAVIIQTHFRAYIFAMKVLASYQKTRSAVIVLQSAYRGMQARKMYIHILTSVIKIQSYYRAYVSKKEFLSLKNATIKLQSTVKMKQTRKQYLHLRAAALFIQQCYRSKKIAAQKREEYMQMRESCIKLQAFVRGYLVRKQMRLQRKAVISLQSYFRMRKARQYYLKMYKAIIVIQNYYHAYKAQVNQRKNFLQVKKAATCLQAAYRGYKVRQLIKQQSIAALKIQSAFRGYNKRVKYQSVLQSIIKIQRWYRAYKTLHDTRTHFLKTKAAVISLQSAYRGWKVRKQIRREHQAALKIQSAFRMAKAQKQFRLFKTAALVIQQNFRAWTAGRKQCMEYIELRHAVLVLQSMWKGKTLRRQLQRQHKCAIIIQSYYRMHVQQKKWKIMKKAALLIQKYYRAYSIGREQNHLYLKTKAAVVTLQSAYRGMKVRKRIKDCNKAAVTIQSKYRAYKTKKKYATYRASAIIIQRWYRGIKITNHQHKEYLNLKKTAIKIQSVYRGIRVRRHIQHMHRAATFIKAMFKMHQSRISYHTMRKAAIVIQVRCRAYYQGKMQREKYLTILKAVKVLQASFRGVRVRRTLRKMQTAATLIQSNYRRYRQQTYFNKLKKITKTVQQRYWAMKERNIQFQRYNKLRHSVIYIQAIFRGKKARRHLKMMHIAATLIQRRFRTLMMRRRFLSLKKTAILIQRKYRAHLCTKHHLQFLQVQNAVIKIQSSYRRWMIRKRMREMHRAATFIQSTFRMHRLHMRYQALKQASVVIQQQYQANRAAKLQRQHYLRQRHSAVILQAAFRGMKTRRHLKSMHSSATLIQSRFRSLLVRRRFISLKKATIFVQRKYRATICAKHKLYQFLHLRKAAITIQSSYRRLMVKKKLQEMQRAAVLIQATFRMYRTYITFQTWKHASILIQQHYRTYRAAKLQRENYIRQWHSAVVIQAAYKGMKARQLLREKHKASIVIQSTYRMYRQYCFYQKLQWATKIIQEKYRANKKKQKVFQHNELKKETCVQAGFQDMNIKKQIQEQHQAAIIIQKHCKAFKIRKHYLHLRATVVSIQRRYRKLTAVRTQAVICIQSYYRGFKVRKDIQNMHRAATLIQSFYRMHRAKVDYETKKTAIVVIQNYYRLYVRVKTERKNFLAVQKSVRTIQAAFRGMKVRQKLKNVSEEKMAAIVNQSALCCYRSKTQYEAVQSEGVMIQEWYKASGLACSQEAEYHSQSRAAVTIQKAFCRMVTRKLETQKCAALRIQFFLQMAVYRRRFVQQKRAAITLQHYFRTWQTRKQFLLYRKAAVVLQNHYRAFLSAKHQRQVYLQIRSSVIIIQARSKGFIQKRKFQEIKNSTIKIQAMWRRYRAKKYLCKVKAACKIQAWYRCWRAHKEYLAILKAVKIIQGCFYTKLERTRFLNVRASAIIIQRKWRAILPAKIAHEHFLMIKRHRAACLIQAHYRGYKGRQVFLRQKSAALIIQKYIRAREAGKHERIKYIEFKKSTVILQALVRGWLVRKRFLEQRAKIRLLHFTAAAYYHLNAVRIQRAYKLYLAVKNANKQVNSVICIQRWFRARLQEKRFIQKYHSIKKIEHEGQECLSQRNRAASVIQKAVRHFLLRKKQEKFTSGIIKIQALWRGYSWRKKNDCTKIKAIRLSLQVVNREIREENKLYKRTALALHYLLTYKHLSAILEALKHLEVVTRLSPLCCENMAQSGAISKIFVLIRSCNRSIPCMEVIRYAVQVLLNVSKYEKTTSAVYDVENCIDILLELLQIYREKPGNKVADKGGSIFTKTCCLLAILLKTTNRASDVRSRSKVVDRIYSLYKLTAHKHKMNTERILYKQKKNSSISIPFIPETPVRTRIVSRLKPDWVLRRDNMEEITNPLQAIQMVMDTLGIPY

SEQ ID NO: 3 - RNA, 4755 nt, Homo sapiens
  source 1..4755: mol_type = mRNA
ggatattggagaagatattccactagacaaagatttctgaaattgaaatattattcaatcatcctgcaatctaggataagaatgataattgctgttacatcttataaacgatatctttgggctacagttacaattcagaggcattggcgtgcttatttaagaagaaaacaagatcaacaaagatatgaaatgctaaaatcatcaactcttataatccaatctatgttcagaaaatggaagcaacgtaaaatgcaatcacaagtaaaagctacagtaatattgcaaagagcttttagagaatggcatttaagaaaacaagctaaagaagaaaattctgctattatcatacaatcatggtatagaatgcataaagaattacggaaatatatttatattagatcttgtgttgttatcattcagaaaagatttcggtgctttcaagcccaaaagttatataaaagaagaaaagagtccatactaaccatccagaagtactacaaagcatatctgaaaggaaagattgagcgcaccaactatttgcagaaacgagctgcagccattcaattacaagctgcttttaggagactgaaagctcataatttatgtagacaaattagagctgcttgtgttattcagtcatactggagaatgagacaagacagagttcgatttttaaaccttaagaagactattatcaaatttcaggcacatgtaagaaaacatcaacaacgacagaaatataagaagatgaagaaagcagctgttataattcagactcatttccgagcttatatttttgccatgaaagttctagcatcttaccagaaaacacgctctgctgtcattgtgctgcagtctgcatatagagggatgcaagccaggaaaatgtatattcacatcctcacatctgttataaagattcaatcatattatcgtgcttatgtttctaaaaaggaatttttgagcctaaaaaatgctacaataaaattgcagtcaactgttaagatgaaacaaacacgtaaacaatatttgcatttaagagcagctgcactatttatccagcaatgttaccgttccaaaaaaatagctgcacaaaagagagaagagtatatgcagatgcgggaatcttgtatcaaactgcaagcatttgttagaggataccttgtccgaaagcagatgaggttacaaagaaaagctgttatttcactacagtcttatttcagaatgagaaaggctcggcagtattatctgaaaatgtataaagcaattattgtcattcagaattactatcatgcatacaaagcacaggtcaatcagaggaagaacttcttgcaagtcaaaaaagcagctacttgcttgcaagcagcttacagaggttataaagtacgccagctaatcaaacaacaatctatagctgctcttaaaattcagtctgcttttagaggctataataaaagggtaaaatatcaatctgtgcttcaatctataataaagattcagagatggtacagggcgtacaagactcttcatgatacaagaacacattttttgaagacaaaggcagctgtgatttccctccagtctgcttatcgtggctggaaggttcggaaacagattagaagggaacatcaagctgccttgaagattcagtctgcttttagaatggccaaggcccagaaacagtttagattgtttaaaacagcagcattagtcatccagcaaaatttcagagcatggactgcaggaaggaagcaatgtatggagtatattgaactccgtcatgcggtactggtgcttcaatctatgtggaagggaaaaacactgagaagacagcttcaaaggcaacataaatgtgctatcatcatacagtcatactatagaatgcatgtgcaacaaaagaagtggaaaatcatgaaaaaagctgctcttctgattcaaaagtattatagggcttacagtattggaagagaacagaatcatttatatttgaaaacaaaagcagctgtagtaactttacagtcagcttatcgtggtatgaaagtgagaaaaagaataaaggattgcaacaaagcagcagtcactatacagtctaaatacagagcttacaaaaccaaaaagaaatatgcaacctatagagcttcagctattataattcagagatggtatcgaggtattaaaattacaaaccatcagcataaggagtatcttaatttgaagaagacagcaattaaaatccaatctgtttatagaggtattagagttagaagacatattcaacacatgcacagggcagccacttttattaaagccatgtttaaaatgcatcagtcaagaataagttaccatacaatgagaaaagcagctattgttattcaagtaagatgtagagcatattatcaaggtaaaatgcagcgtgaaaagtacctgacaattttgaaagctgttaaagtccttcaggcaagttttagaggagtaagagttagacggactcttagaaagatgcagactgcagcaacactcattcagtcaaactacagaagatacagacagcaaacatactttaataagttaaagaaaataacaaaaacagtacagcaaagatactgggcaatgaaagaaagaaacatacaatttcaaaggtataacaaactgaggcattctgtaatatacattcaggctatttttaggggaaagaaagctagaagacatttaaaaatgatgcatatagccgcaactctcattcagaggagatttagaactctaatgatgagaagaagattcctctctctcaagaaaactgctattttgattcagagaaaatatcgggcacatctttgtacaaagcatcacttacagttccttcaggtacaaaatgcagttattaaaatccagtcatcatacagaagatggatgataaggaaaaggatgcgagagatgcacagggctgctactttcatccagtctactttcagaatgcacagattacatatgagatatcaggctttgaaacaggcctccgttgtgatccaacagcaataccaagcaaatagagctgcaaaactgcagaggcagcattatctcagacaaagacactctgctgtgatccttcaggctgcattcaggggtatgaaaactagaagacatttgaagagtatgcattcctctgcaacccttattcagagtaggtttagatcattactggtgaggagaagattcatttccctcaaaaaagctactatttttgttcagaggaaatatcgagccaccatttgtgccaaacataaattgtaccaattcttgcacttaagaaaggcagccattacaatacagtcatcttacagaagactgatggtaaagaagaagttacaagaaatgcaaagggctgcagttctcattcaggctactttcaggatgtacagaacatatattacatttcagacttggaaacatgcttcaattctaattcagcaacattatcgaacatatagagctgcaaaattacaaagagaaaattatatcagacaatggcattctgctgtggttattcaggctgcatataaaggaatgaaagcaagacaacttttaagggaaaaacacaaagcttctatcgtaatacaaagcacctacagaatgtataggcagtattgtttctaccaaaagcttcagtgggctacaaaaatcatacaagaaaaatatagagcaaataaaaagaaacagaaagtatttcaacacaatgaacttaagaaagagacttgtgttcaggcaggttttcaggacatgaacataaaaaaacagattcaggaacagcaccaggctgccattattattcagaagcattgtaaagcctttaaaataaggaagcattatctccaccttagagcaacagtagtttctattcaaagaagatacagaaaactaactgcagtgcgtacccaagcagttatttgtatacagtcttattacagaggctttaaagtacgaaaggatattcaaaatatgcaccgggctgccacactaattcagtcattctatcgaatgcacagggccaaagttgattatgaaacaaagaaaactgcaattgtggttatacagaattattataggttgtatgttagagtaaaaacagaaagaaaaaactttttagcagttcagaaatctgtacgaactattcaggctgcttttagaggcatgaaagttagacaaaaattgaaaaatgtatcagaggaaaagatggcagccattgttaaccaatctgcactctgctgttacagaagtaaaactcagtatgaagctgttcaaagtgaaggtgttatgattcaagagtggtataaagcttctggccttgcttgttcacaggaagcagagtatcattctcaaagtagggctgcagtaacaattcaaaaagctttttgtagaatggtcacaagaaaactggaaacacagaaatgtgctgccctacggattcagttcttccttcagatggctgtgtatcggagaagatttgttcagcagaaaagagctgctatcactttacagcattattttaggacgtggcaaaccagaaaacagtttttactatatagaaaagcagcagtggttttacaaaatcactacagagcatttctgtctgcaaaacatcaaagacaagtctatttacagatcagaagcagtgttatcattattcaagctagaagtaaaggatttatacagaaacggaagtttcaggaaattaaaaatagcaccataaaaattcag

SEQ ID NO: 4 - RNA, 19 nt, synthetic construct
  source 1..19: mol_type = other RNA
gcgcaccaactatttgcag

SEQ ID NO: 5 - RNA, 19 nt, synthetic construct
  source 1..19: mol_type = other RNA
ctgcaaatagttggtgcgc

SEQ ID NO: 6 - RNA, 19 nt, synthetic construct
  source 1..19: mol_type = other RNA
cagaagactgatggtaaag

SEQ ID NO: 7 - RNA, 19 nt, synthetic construct
  source 1..19: mol_type = other RNA
ctttaccatcagtcttctg

SEQ ID NO: 8 - RNA, 19 nt, synthetic construct
  source 1..19: mol_type = other RNA
cctgcaatctaggataaga

SEQ ID NO: 9 - RNA, 19 nt, synthetic construct
  source 1..19: mol_type = other RNA
ggacgttagatcctattct